(19) United States
(12) Patent Application Publication
(10) Pub. No.: US 2021/0147466 A1
(43) Pub. Date: May 20, 2021

(54) IMPROVED PROCESSES FOR THE PREPARATION OF GUADECITABINE AND INTERMEDIATES THEREOF

(71) Applicant: TEVA CZECH INDUSTRIES s.r.o., Opava-Komarov (CZ)

(72) Inventors: Jana ROHACOVA, Ostrava-Jih (CZ); Jiri ZUREK, Opava-Komarov (CZ); Hana KANTOR, Ostrava (CZ); Ales HUSEK, Opava (CZ)

(21) Appl. No.: 16/623,571

(22) PCT Filed: Jul. 11, 2018
(86) PCT No.: PCT/US2018/041538

§ 371 Date: Dec. 17, 2019

Related U.S. Application Data
(60) Provisional application No. 62/590,747, filed on Nov. 27, 2017.
(60) Provisional application No. 62/531,068, filed on Jul. 11, 2017.

(51) Int. Cl. C07H 19/207; C07C 279/02; C07H 19/12

(52) U.S. Cl. CPC C07H 19/207; C07B 2200/13; C07H 19/12; C07C 279/02

(57) ABSTRACT

Described are procedures and intermediates for the preparation of guadecitabine, and guadecitabine salts and solid state forms thereof.

### FIELD OF THE INVENTION

The present invention provides new procedures and intermediates for the preparation of guadecitabine.

### BACKGROUND OF THE INVENTION

Guadecitabine, [(2R,3S,5R)-5-(2-amino-6-oxo-3H-purin-9-yl)-3-hydroxyoxolan-2-yl]methyl [(2R,3S,5R)-5-(4-amino-2-oxo-1,3,5-triazin-1-yl)-2-(hydroxymethyl)oxolan-3-yl] hydrogen phosphate referred to herein as compound 1, has the following chemical structure:

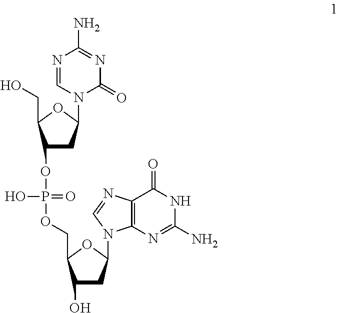 [SMILES: Nc1ncn([C@H]2C[C@@H](OP(=O)(O)OC[C@H]3O[C@@H](n4cnc5c(=O)[nH]c(N)nc54)C[C@H]3O)[C@@H](CO)O2)c(=O)n1]

Guadecitabine is a DNA methyltransferase (DNMT) inhibitor. It is a hypomethylating agent (HMA) designed as a dinucleotide of decitabine and deoxyguanosine that is resistant to deamination by cytidine deaminase (CDA). Guadecitabine is being tested in clinical trials for the treatment of solid tumors.

Guadecitabine is described in U.S. Pat. No. 7,700,567 wherein the preparation of guadecitabine by solid phase synthesis and in solution is disclosed. The processes can be schematically illustrated as depicted in Scheme 1 and Scheme 2 respectively.

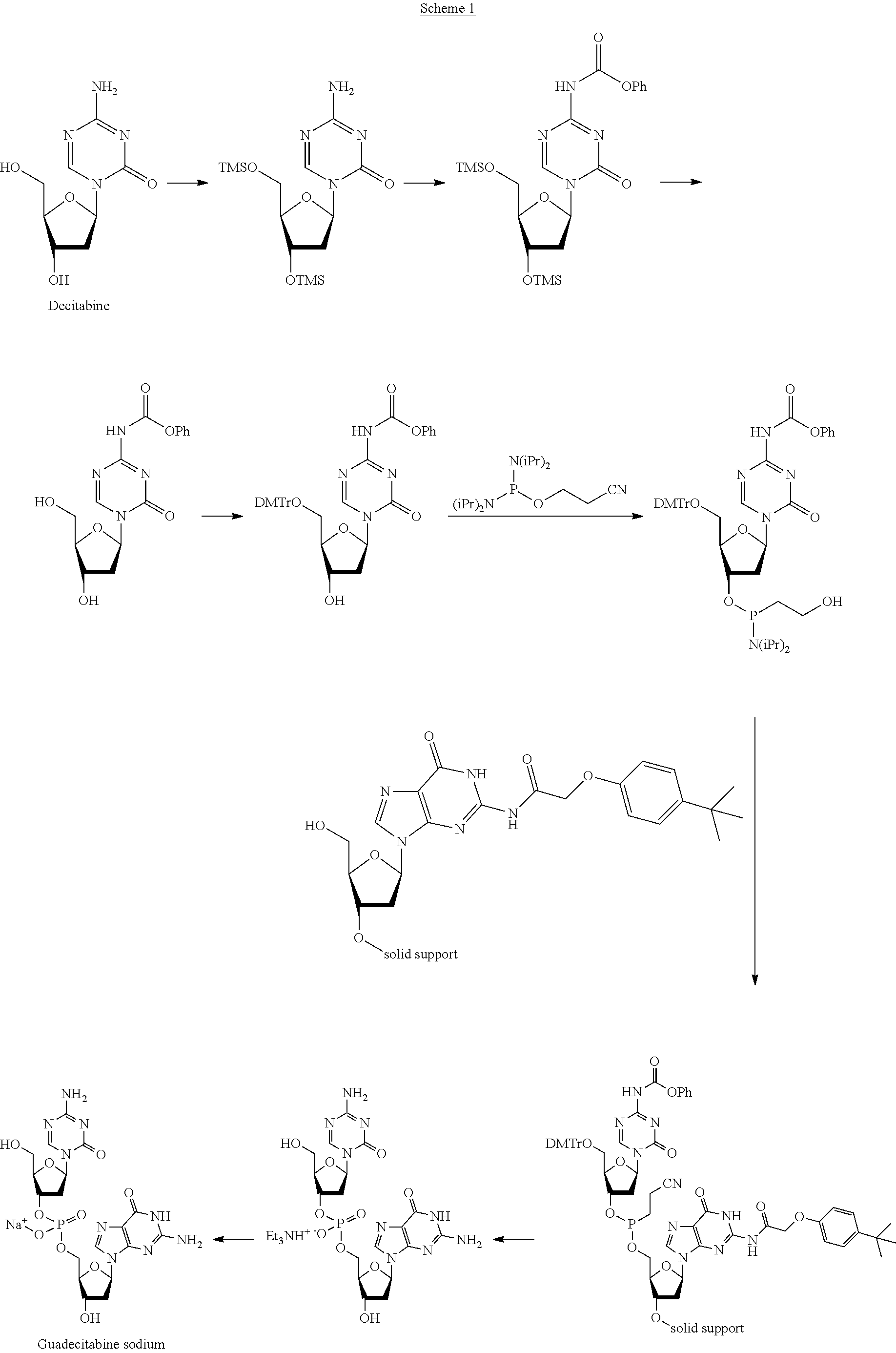

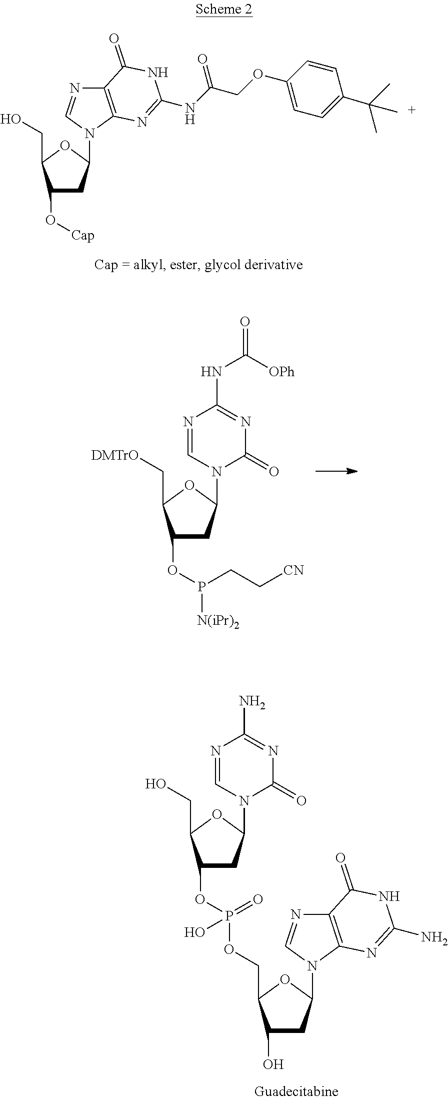

According to the disclosure in U.S. Pat. No. 7,700,567, Guadecitabine is synthesized on solid support by coupling N-protected (by tert-butyl phenoxy acetyl) 2′-deoxyguanosine-linked CPG solid support with phenoxyacetyl decitabine phosphoramidite, followed by removal of the base protecting groups, oxidation of the coupled product, detritylation and removal from the solid support. In a similar manner, in solution the synthesis includes coupling of N-protected 3′-O-capped 2′-deoxyguanosine with phenoxyacetyl decitabine phosphoramidite, followed by oxidation of the coupled product and removal of the amine protecting groups and detritylation.

There is still a need in the art to provide an efficient process utilizing less number of synthetic steps, which provides guadecitabine in high yield and quality, and that can be utilized in industrial scale.

### SUMMARY OF THE INVENTION

The present disclosure provides a novel process for preparation of Guadecitabine.

The disclosure further provides novel intermediates that can be advantageously used for preparation of Guadecitabine and processes for the preparation of the intermediates.

In another aspect the present disclosure provides the use of any one of the compounds of novel intermediates for the preparation of Guadecitabine.

In another aspect the present disclosure provides any one of the novel intermediates for use in the preparation of Guadecitabine.

In another aspect the present disclosure provides Guadecitabine produced by the processes of the present disclosure.

Guadecitabine produced by the processes of the present disclosure may be used to prepare salts of Guadecitabine.

Guadecitabine or Guadecitabine salts produced by the processes of the present disclosure may be used in the preparation of pharmaceutical compositions of Guadecitabine or Guadecitabine salts.

The present disclosure also encompasses the use of the Guadecitabine or Guadecitabine salts prepared by the processes of the present disclosure for the preparation of pharmaceutical compositions of Guadecitabine or Guadecitabine salts.

The present disclosure comprises processes for preparing the above mentioned pharmaceutical compositions. The processes comprise combining the Guadecitabine prepared by the processes of the present disclosure or salts thereof with at least one pharmaceutically acceptable excipient.

Guadecitabine or salts thereof prepared by the processes of the present disclosure and the pharmaceutical compositions of Guadecitabine or salts thereof prepared by the processes of the present disclosure can be used as medicaments, particularly for the treatment of cancer.

The present disclosure also provides methods for the treatment of cancer, comprising administering a therapeutically effective amount of Guadecitabine or salts thereof prepared by the processes of the present disclosure, or at least one of the above pharmaceutical compositions, to a subject in need of the treatment.

### BRIEF DESCRIPTION OF THE DRAWINGS

FIG. 1 shows the X-ray powder diffraction pattern of guadecitabine triethylammonium salt obtained by example 7.

FIG. 2 shows the X-ray powder diffraction pattern of crystalline 5′-O-tert-butyldimethylsilyl-2′-deoxy-5-azacytidine (7a) obtained by example 4.

FIG. 3 shows the X-ray powder diffraction pattern of crystalline 5′-O-tert-butyldimethylsilyl-2′-deoxy-5-azacytidine (7a) obtained by example 4-zoomed.

FIG. 4 shows an X-ray powder diffraction (XRPD) pattern of Form G1 of Guadecitabine guanidinium.

FIG. 5 shows an X-ray powder diffraction (XRPD) pattern of Form A of Guadecitabine sodium.

FIG. 6 shows an X-ray powder diffraction (XRPD) pattern of Form B of Guadecitabine sodium.

FIG. 7 shows an X-ray powder diffraction (XRPD) of 3′,5′-bis-O-(tert-butyldimethylsilyl)-2′-deoxyguanosine (3a) obtained by example 12.

FIG. 8 shows an X-ray powder diffraction (XRPD) of 3′-O-tert-butyldimethylsilyl-2′-deoxyguanosine (4a) obtained by example 13.

FIG. 9 shows an X-ray powder diffraction (XRPD) of 3′-O-tert-butyldimethylsilyl-2′-deoxyguanosine (4a) obtained by example 13 (zoomed).

FIG. 10 shows an X-ray powder diffraction (XRPD) of 5′-O-tert-butyldimethylsilyl-2′-deoxy-5-azacytidine (7a) obtained by example 15.

FIG. 11 shows an X-ray powder diffraction (XRPD) of 5′-O-tert-butyldimethylsilyl-2′-deoxy-5-azacytidine (7a) obtained by example 15 (zoomed).

FIG. 12 shows an X-ray powder diffraction (XRPD) of 5′-O-tert-butyldimethylsilyl-2′-deoxy-5-azacytidine-3′-O-[(2-cyanoethyl)-N,N-diisopropylphosphoramidite] (9a) obtained by example 16.

FIG. 13 shows an X-ray powder diffraction (XRPD) of 2-Cyanoethyl (5′-O-(tert-butyldimethylsilyl)-2′-deoxy-5-azacytosylyl)-(3′→5′)-3′-O-(tert-butyldimethylsilyl)-2′-deoxyguanosine (11a) obtained by example 17.

FIG. 14 shows an X-ray powder diffraction (XRPD) of semi crystalline form G1 of guadecitabine guanidinium obtained by example 19.

FIG. 15 shows an X-ray powder diffraction (XRPD) of form A of guadecitabine sodium obtained by example 20.

FIG. 16 shows an FTIR spectrum of form A of guadecitabine sodium.

FIG. 17 shows an FTIR spectrum of form A of guadecitabine sodium (zoomed).

FIG. 18 shows a 13C solid state NMR spectrum of form A of guadecitabine sodium obtained by example 20 (range from 200-0 ppm).

FIG. 19 shows a ¹³C solid state NMR spectrum of form A of guadecitabine sodium obtained by example 20 (range from 200-100 ppm).

FIG. 20 shows an X-ray powder diffraction (XRPD) crystalline form G2 of guadecitabine guanidinium obtained by example 22.

FIG. 21 shows Scheme 3.

### DETAILED DESCRIPTION OF THE INVENTION

The present disclosure provides new processes and intermediates for the preparation of guadecitabine.

As discussed earlier, the processes described in the literature have significant disadvantages. The process disclosed in U.S. Pat. No. 7,700,567 includes protection of the amine groups of the nucleoside bases prior to the coupling step. Deprotection is required after coupling in order to afford Guadecitabine. Decitabine is unstable in aqueous media and undergoes hydrolytic degradation, therefore significant degradation occurs during the amine deprotection reaction which is performed in basic pH and in the presence of other nucleophiles. Another disadvantage is purification by chromatography on C18 column using aqueous phase, in which guadecitabine is not stable.

In contrast to the prior art processes, the processes of the present disclosure do not involve use of protecting groups to protect the nucleobases. The process of the present disclosure employs the “proton block” approach for preparation of guadecitabine. The process involves protection of the nucleobases of decitabine and deoxyguanosine with protons thereby rendering them unreactive during condensation. This method avoids the base protection and deprotection steps, and therefore provides an efficient process for preparation of guadecitabine. More importantly, the degradation of decitabine is avoided, offering a process of high yield an high purity that can be adapted to production in an industrial scale, i.e., greater than 1 kilogram scale.

Prior publications disclose preparation of guadecitabine sodium by dissolution of guadecitabine triethylammonium salt in water followed by addition of sodium perchlorate using sodium perchlorate in water. Based on the literature decitabine undergoes decomposition in the presence of water. Since guadecitabine possesses a decitabine moiety, several degradation products may be formed in the presence of water and are depicted in scheme 3. It has been surprisingly found that guadecitabine degradation is significantly suppressed in methanol. Thus, the preparation of guadecitabine sodium according to the invention in a solvent system comprising methanol and a low amount of water offers highly pure guadecitabine sodium.

Further, the prior art process uses sodium perchlorate for conversion of guadecitabine triethylammonium salt to guadecitabine sodium, while the process of the present invention involves use of sodium acetate for the preparation of guadecitabine sodium. Perchlorate is difficult to remove and requires monitoring the residual content in the final API which is a serious drawback. Thus use of sodium acetate is advantageous and suitable for industrial scale processes.

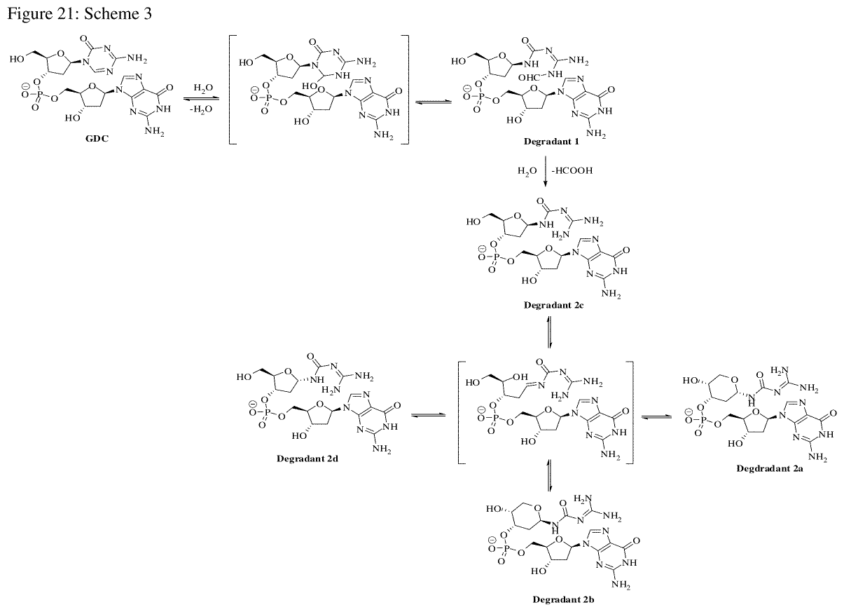

Moreover, it has been surprisingly found that guadecitabine guanidinium offers significant impurity purging capability in that the purity of guadecitabine guanidinium after conversion from guadecitabine triethylammonium salt having HPLC purity of 98.1 wt % was upgraded to 99.8 wt % and thus chromatographic purification is avoided. Thus, the process of the present invention provides guadecitabine sodium in overall high yield of about 35-50% (calculated on starting decitabine) and high quality, i.e. high chemical purity. Specifically, the process of the present invention provides guadecitabine sodium which contains about 0.15 wt % or less, preferably about 0.10 wt % or less, more preferably 0.05 wt % or less of each one of degradant 2a and degradant 2b and/or contains about 0.25 wt % or less, preferably about wt 0.20% or less, more preferably 0.15 wt % or less of each one of degradant 2c and degradant 2d, the structures of which are depicted in Scheme 3 (FIG. 21).

As used herein, and unless indicated otherwise, the term “isolated” in reference to the intermediates of the present disclosure, their salts or solid state forms thereof corresponds to compounds that are physically separated from the reaction mixture in which they are formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The processes or steps may be referred to herein as being carried out “overnight.” This refers to time intervals, e.g., for the processes or steps, that span the time during the night, when the processes or steps may not be actively observed. The time intervals are from about 8 to about 20 hours, or about 10 to about 18 hours, or about 16 hours.

As used herein, and unless indicated otherwise, the term “reduced pressure” refers to a pressure of about 10 mbar to about 500 mbar, or about 50 mbar.

As used herein, and unless indicated otherwise, the term “chlorinated solvent” refers to a C1-C6 chlorinated hydrocarbon. In some embodiments, the chlorinated solvents are selected from the group consisting of carbon tetrachloride, dichloromethane (CH₂Cl₂), dichloroethane, chlorobenzene, and chloroform.

As used herein, and unless indicated otherwise, the term “one pot process” refers to continues process for preparing a desired product, in which penultimate product is converted to the desired product in the same vessel.

As used herein, and unless indicated otherwise, the term “Protecting group” refers to a grouping of atoms that when attached to a reactive functional group in a molecule masks, reduces or prevents reactivity of the functional group. Examples of protecting groups can be found in Greene and Wuts “Greene's Protective Groups in Organic Synthesis”, 4th Edition, publ. Wiley, 2006 and Harrison et al., “Compendium of Synthetic Organic Methods”, Vols. 1-8 (John Wiley and Sons, 1971-1996).

Representative amine protecting groups include, but are not limited to, those where the amine group is converted to carbamate or amide such as Fmoc, cbz, benzyl, trityl, Boc, trifluoroacetyl derivative, phthalic anhydride, or succinic anhydride derivative.

Representative hydroxyl protecting groups include, but are not limited to, those where the hydroxyl group is converted to ethers, esters, carbonates, carbamates, siloxanes, or acetals.

The amount of solvent employed in chemical processes, e.g., reactions or crystallizations, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding MTBE (1.5 v/v) to a 100 ml reaction mixture would indicate that 150 mL of MTBE was added.

As used herein, “halogen” or “halide” refers to fluoride, chloride, bromide or iodide. In certain embodiments, the halogen is bromide or iodide.

“Alkyl” refers to a monoradical of a branched or unbranched saturated hydrocarbon chain and can be substituted or unsubstituted. Lower alkyl groups may contain 1-6 carbon atoms or 1-4 carbon atoms. Examples of alkyl groups include, but are not limited to, methyl, ethyl, n-propyl, n-butyl, n-pentyl, n-hexyl, n-heptyl, n-octyl, n-nonyl, n-decyl, isopropyl, tert-butyl, isobutyl, etc.

“Alkoxy” refers to the O-(alkyl) group where the alkyl group is defined above.

“Aryl” refers to phenyl and 7-15 membered monoradical bicyclic or tricyclic hydrocarbon ring systems, including bridged, spiro, and/or fused ring systems, in which at least one of the rings is aromatic. Aryl groups can be substituted or unsubstituted. Examples include, but are not limited to, naphthyl, indanyl, 1,2,3,4-tetrahydronaphthalenyl, 6,7,8,9-tetrahydro-5H-benzocycloheptenyl, and 6,7,8,9-tetrahydro-5H-benzocycloheptenyl. An aryl group may contain 6 (i.e., phenyl) or 9 to 15 ring atoms, such as 6 (i.e., phenyl) or 9-11 ring atoms, e.g., 6 (i.e., phenyl), 9 or 10 ring atoms.

A solid state form according to the present disclosure may have advantageous properties selected from at least one of: chemical or polymorphic purity, flowability, solubility, dissolution rate, bioavailability, morphology or crystal habit, stability—such as chemical stability as well as thermal and mechanical stability with respect to polymorphic conversion, stability towards dehydration and/or storage stability, a lower degree of hygroscopicity, low content of residual solvents, adhesive tendencies and advantageous processing and handling characteristics such as compressibility, and bulk density.

A crystal form may be referred to herein as being characterized by graphical data “as depicted in” a Figure. Such data include, for example, powder X-ray diffractograms and solid state NMR spectra. As is well-known in the art, the graphical data potentially provides additional technical information to further define the respective solid state form (a so-called “fingerprint”) which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms.

A crystal form referred to herein as being characterized by graphical data “as depicted in” a Figure will thus be understood to include any crystal forms of the compound, characterized with the graphical data having such small variations, as are well known to the skilled person, in comparison with the Figure.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, solid state of guadecitabine or salt thereof described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject solid state form of guadecitabine or salt thereof. Accordingly, in some embodiments of the disclosure, the described solid state form of guadecitabine or salt thereof may contain from about 1% to about 20% (w/w), from about 5% to about 20% (w/w), or from about 5% to about 10% (w/w) of one or more other solid state forms of the same guadecitabine or salt thereof.

As used herein, unless stated otherwise, XRPD peaks reported herein are preferably measured using CuKα radiation, λ=1.54187 Å. XRPD peaks reported herein are measured using CuKα radiation, λ=1.54187 Å, at a temperature of 25±3° C.

As used herein, unless stated otherwise, chemical purity (wt %) may be measured by HPLC analysis. Preferably, the HPLC analysis is carried out using a reversed phase silica gel column (e.g. C18 column) using UV detection at 249 nm. Any suitable eluent can be used to carry out the separation (preferably a mixture of acetonitrile/water is used). The HPLC analytical methods are designed to use UV absorption at a given wavelength for recording the presence and the amount of a compound in a sample passing the detector at any given point in time. For example, the primary output of any HPLC run with standard equipment will be an area percentage of the respective peak in the UV detection chromatogram, i.e., the area under the curve (AUC).

Those skilled in the art understand that the area % values can by equated with a “% by weight” value (wt %) in cases where the response factors of the drug substance and the relevant impurity are close. In opposite case this practice can still be appropriate, provided a correction factor is applied, in line with ICH guidelines (IMPURITIES IN NEW DRUG SUBSTANCES Q3A(R2)].

The content of an impurity Xi is calculated according to the formula:

$${x}_{i} = \frac{\frac{{AREA}_{{x}_{i}}}{{RRF}_{{x}_{i}}}}{AREA + {\sum }_{i = 1}^{n} \frac{{Area}_{{x}_{i}}}{{RRF}_{{x}_{i}}}} \times 100$$

where

AREAXi corresponds to peak area of impurity Xi

AREA corresponds to peak area of guadecitabine

RRFxi corresponds to relative response factor of impurity Xi.

The present disclosure provides novel intermediates of formulae A, B, 5, 7, 9, 10 and 11 that may be advantageously used for the preparation of guadecitabine.

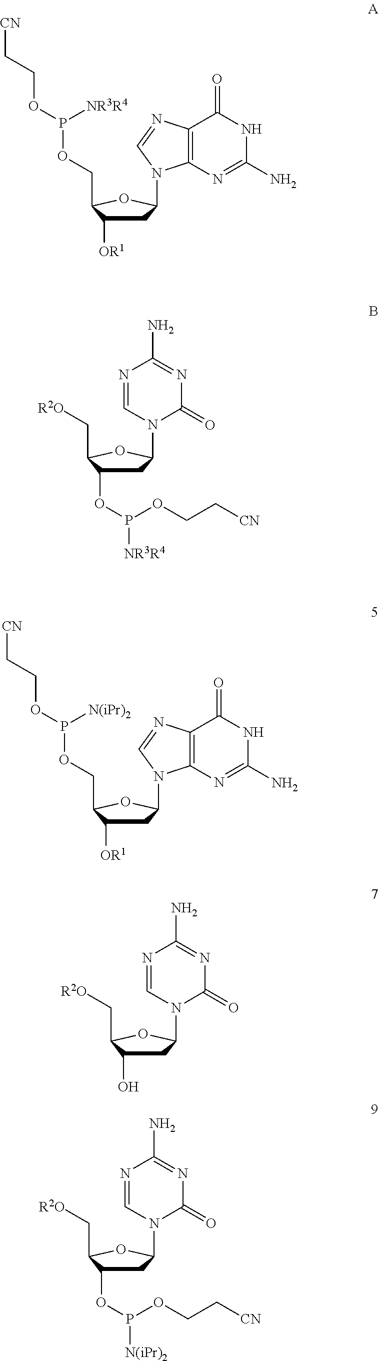

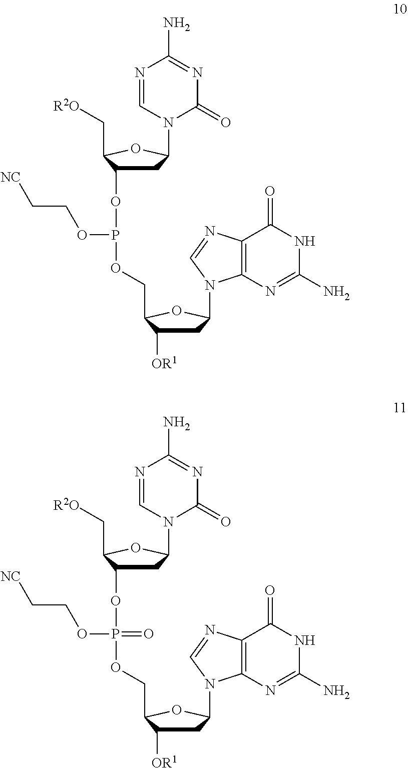

wherein R1 and R2 may be the same or different suitable hydroxy protecting group. Suitable hydroxyl protecting group can be found in Greene and Wuts “Greene's Protective Groups in Organic Synthesis”, 4th Edition, publ. Wiley, 2006.

Suitable hydroxy protecting groups may include, for example silyl-type protecting groups according to the formula —SiR7R8R9, wherein R7, R8 and R9 are independently selected from the group consisting of: C1-C15 straight or branched alkyl, C1-C10 cycloalkyl, optionally substituted C6-C10 aryl, and optionally substituted C7-C12 arylalkyl. Preferably, the silyl-type protecting groups may be t-butyldimethylsilyl (TBDMS), triethylsilyl (TES), t-butyldiphenylsilyl (TBDPS), triisopropylsilyl (TIPS), and trimethylsilyl (TMS). Other suitable hydroxyl-protecting groups may include for example, methyl or substituted methyl groups, preferably, tetrahydropyranyl (THP), methoxymethyl (MOM), benzyloxymethyl; ethyl or substituted ethyl groups, preferably, ethoxyethyl, benzyl or tert-butyl; ester groups, preferably acetate or aryl substituted acetate groups, for example phenoxyacetate (pac), tert-butylphenoxyacetate (tac), benzoate or substituted benzoate groups; carbonates, preferably benzyloxycarbonate (CBZ), tert-butyloxycarbonate (BOC), fluorenylmethoxycarbonate (FMOC); acetals, preferably methoxymethyl (MOM), 2-methoxyethoxymethyl (MEM), benzyloxymethyl, or tetrahydropyranyl (THP). In a preferred embodiment, R1 and R2 are TBDMS. Additional hydroxyl-protecting groups may be selected from those described in Greene's Protective Groups in Organic Synthesis.

R3 and R4 may be the same or different and are selected from the group consisting of hydrogen, methyl, ethyl, n-propyl, isopropyl, trimethylsilyl, or R3 and R4 taken together with the intervening nitrogen atom form an optionally substituted heterocyclyl such as pyrrolidine, piperidine, morpholine, 2,2,6,6-tetramethylpiperidine, imidazole, 4,5-dichloroimidazole, triazole, or tetrazole. Preferably R3, R4, are isopropyl.

In some embodiments the present disclosure provides compounds 5a, 7a, 9a, 10a and 11a of the following structures:

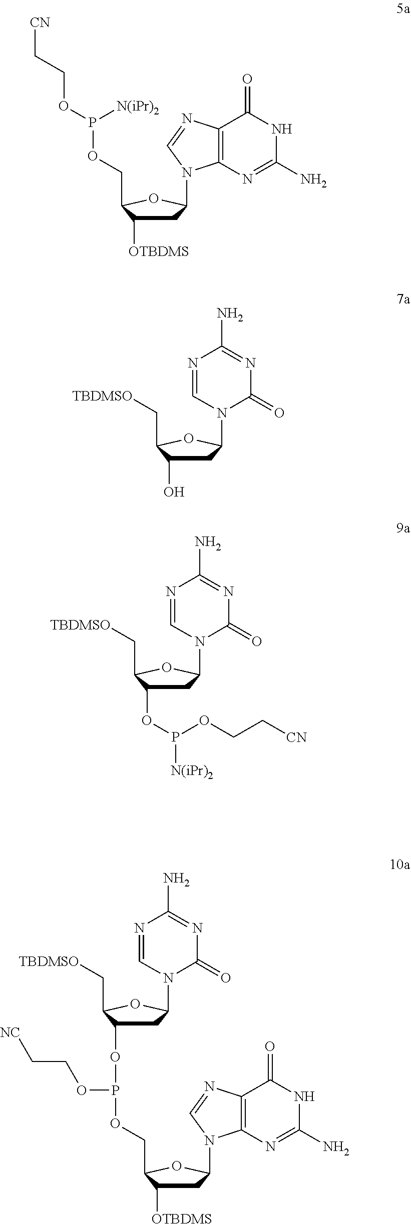

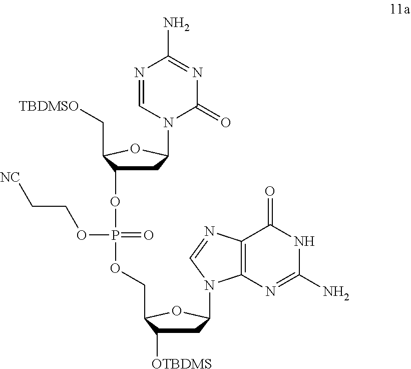

In certain embodiments any one of compounds 5a, 7a, 9a, 10a and 11a is isolated in solid form.

In another aspect the present disclosure provides the use of any one of the compounds of formulae A, B, 5, 7, 9, 10 and 11 in the preparation of guadecitabine.

In some embodiments, the disclosure provides the use of any one of compounds 5a, 7a, 9a, 10a and 11a as described above for the preparation of guadecitabine.

In another aspect the present disclosure provides any one of compounds of formulae A, B, 5, 7, 9, 10 and 11 for use in the preparation of guadecitabine.

In some embodiments the disclosure provides any one of compounds of formulae 5a, 7a, 9a, 10a and 11a for use in the preparation of guadecitabine.

The present disclosure provides for novel processes for preparation of any one of compounds A, B, 5, 7, 9, 10 and 11 and for the preparation of guadecitabine.

In one aspect, the present disclosure provides a process for preparation of guadecitabine or salts thereof comprising:
a) oxidation of compound 10

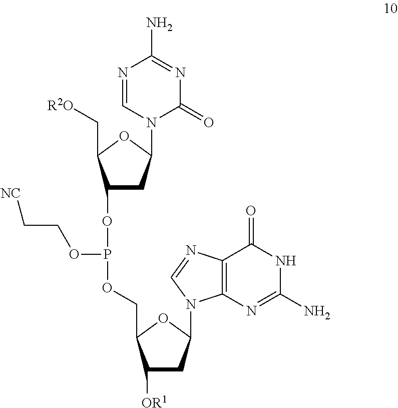

to afford compound 11

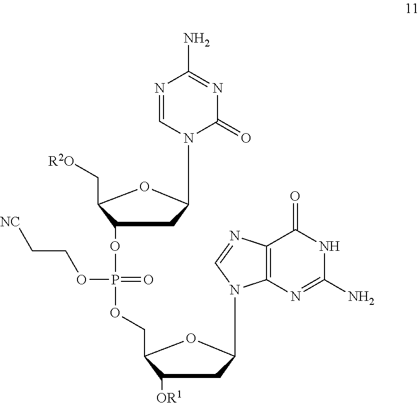

wherein R¹ and R² are as defined above; and
b) conversion of compound 11 to guadecitabine (compound 1) or salts thereof.

Step a) is typically carried out in the presence of a suitable solvent, and a suitable oxidation reagent. Suitable solvents may include, for example, dimethylformamide (DMF), dimethylacetamide (DMA), N-methyl-2-pyrrolidone (NMP), dimethylsulphoxide (DMSO), acetonitrile, toluene, methyl tert-butyl ether (MTBE), n-heptane, n-hexane, dichloromethane (DCM), n-nonane, 1,4-dioxane or mixtures thereof. Preferably, the solvent is 1,4-dioxane, DMF, acetonitrile or combination thereof. More preferably, the solvent is a combination of acetonitrile and DMF. In another embodiment, the reaction is performed in DMF.

Suitable oxidation reagents include but are not limited to iodine, peroxides, peroxoacids, hydroperoxides, halogens or mixtures thereof. Preferably iodine and hydroperoxide, most preferably t-butyl hydroperoxide (t-BuOOH) are used.

Preferably the reaction is performed in anhydrous conditions.

Conversion of compound 11 to guadecitabine or salts thereof typically includes deprotection of the hydroxy groups and hydrolysis of the cyanoethyl group, i.e. deprotection of phosphate group. The hydroxy groups deprotection may be performed before or after the phosphate deprotection, or all protecting groups can be cleaved concurrently.

In a particular embodiment, step b) comprises the following steps:
i) deprotecting compound 11;
ii) formation of a basic salt of guadecitabine and optionally isolating the salt; and
iii) optionally converting the basic salt formed in step ii) to guadecitabine or other basic salt thereof.

Step i) is typically carried out in the presence of a suitable solvent, a suitable base and a fluoride reagent. Suitable solvents may include, for example, DMA (dimethylacetamide), DMF, acetonitrile (AcN), NMP, DMSO, toluene, DCM, MeOH, EtOH, i-PrOH, THF, n-hexane, n-heptane or mixture thereof. Preferably, the solvent is DMA (dimethylacetamide), DMF, DMSO, acetonitrile, toluene, DCM, tetrahydrofuran (THF) or combination thereof. More preferably, the solvent is acetonitrile or DMA (dimethylacetamide). In a particular embodiment the solvent system used is a mixture of DMA and DMSO. The ratio of DMA:DMSO is about 4:1 v/v, preferably about 6:1 v/v to about 10:1 v/v, more preferably about 20:1 v/v to about 30:1 v/v.

Suitable bases include but are not limited to Et₃N, diisopropylethylamine, 1,4-diazabicyclo[2.2.2]octane (DABCO), pyridine, 1,8-diazabicyclo[5.4.0]undec-7-ene (DBU), 1,5-diazabicyclo[4.3.0]non-5-ene (DBN), K₂CO₃ and other amines and other carbonates. Preferably Et₃N or diisopropylethylamine may be used, most preferably triethylamine is used.

Suitable fluoride reagents include but are not limited to pyridine hydrogen fluoride (Py.HF), Tetrabutylammonium fluoride (TBAF), tris(dimethylamino)sulfonium difluorotrimethylsilicate (TASF), Et₃N*3HF, CsF. Other salts of organic base and hydrogenfluoride may be used as well. Preferably may be used TBAF and Et₃N*3HF. Most preferably Et₃N*3HF is used.

Preferably in step ii) triethylamine salt of guadecitabine is formed.

In a specific embodiment, in step iii) guadecitabine triethylamine salt may be converted to another salt of guadecitabine, preferably guadecitabine Na or guadecitabine guanidinium.

In a specific embodiment, guadecitabine Na formed may be crystalline, preferably it may be crystalline form A or crystalline form B.

In a specific embodiment guadecitabine guanidinium formed may be crystalline, preferably it may be crystalline form G1, or form G2.

In another particular embodiment, step b) comprises the following steps:
iv) deprotecting compound 11;
v) formation of a basic salt A of guadecitabine and optionally isolating the salt;
vi) optionally converting the basic salt A formed in step v) to a basic salt B; and
vii) optionally converting the basic salt B formed in step vi) to guadecitabine or another basic salt thereof.

Step iv) is typically carried out in the presence of a suitable solvent, a suitable base and a fluoride reagent. Suitable solvents may include, for example, DMA (dimethylacetamide), DMF, DMSO, acetonitrile (AcN), NMP, toluene, DCM, MeOH, EtOH, i-PrOH, THF, n-hexane, n-heptane or mixture thereof. Preferably, the solvent is DMA (dimethylacetamide), DMF, DMSO, acetonitrile, toluene, DCM, tetrahydrofuran (THF) or combination thereof. More preferably, the solvent is acetonitrile or DMA (dimethylacetamide). In a particular embodiment the solvent system used is a mixture of DMA and DMSO. The ratio of DMA:DMSO is about 4:1 v/v, preferably about 6:1 v/v to about 10:1 v/v, more preferably about 20:1 v/v to about 30:1 v/v.

Suitable bases include but are not limited to Et₃N, diisopropylethylamine, 1,4-diazabicyclo[2.2.2]octane (DABCO), pyridine, 1,8-diazabicyclo[5.4.0]undec-7-ene (DBU), 1,5-diazabicyclo[4.3.0]non-5-ene (DBN), K₂CO₃ and other amines and other carbonates. Preferably Et₃N or diisopropylethylamine may be used, most preferably triethylamine is used.

Suitable fluoride reagent include but are not limited to pyridine hydrogen fluoride (Py.HF), Tetrabutylammonium fluoride (TBAF), tris(dimethylamino)sulfonium difluorotrimethylsilicate (TASF), Et3N*3HF, CsF. Other salts of organic base and hydrogen fluoride may be used.

Preferably may be used TBAF and Et3N*3HF. Most preferably Et3N*3HF is used.

Preferably in step v) triethylamine salt of guadecitabine is formed as the basic salt A.

Preferably in step vi) guadecitabine triethylamine salt may be converted to guadecitabine guanidinium salt as the basic salt B.

Preferably in step vii) guadecitabine guanidinium salt may be converted to guadecitabine Na.

In a specific embodiment, guadecitabine guanidinium formed in step vi) may be crystalline, semi crystalline or amorphous, preferably it may be crystalline form G1, or form G2.

In a specific embodiment guadecitabine Na formed in step vii) may be crystalline, preferably it may be crystalline form A or crystalline form B.

In another aspect the disclosure provides a process for preparation of guadecitabine sodium, preferably crystalline guadecitabine sodium wherein the process comprises:
a) providing guadecitabine triethylamine salt optionally in the form of solution;
b) adding guanidine or guanidinium salt, optionally in the form of solution, to form a suspension;
c) optionally separating guadecitabine guanidinium;
d) providing guadecitabine guanidinium, optionally in the form of solution, and adding a sodium salt, optionally in the form of solution;
e) adding antisolvent; and
f) optionally isolating guadecitabine sodium.
   The process may include additional washing and drying steps.

Examples of suitable guanidinium salts that may be used in step b) include, but are not limited to carbonate, acetate, chloride, nitrate, thiocyanate and sulfate salts. Preferably the guanidinium salt added in step b) is guanidinium carbonate, more preferably in the form of a solution in a solvent system comprising a polar protic solvent, preferably water. Preferably in step d) guadecitabine guanidinium is dissolved or suspended in a solvent system comprising one or more polar protic solvents, such as alcohol and/or water, preferably the solvent system comprises a mixture of alcohol and water, preferably methanol and water, wherein the amount of water is not more than (NMT) about 50% v/v, NMT about 20% v/v, NMT about 10% v/v, or NMT about 5% v/v, preferably wherein the amount of water is NMT about 30% v/v, more preferably NMT about 20% v/v, even more preferably NMT about 10% v/v, and most preferably from about 5% v/v to about 10% v/v.

Organic or inorganic sodium salts may be used in step d). Examples of suitable salts include, but are not limited to, acetate, formate, propionate, nitrate and perchlorate salts. Preferably the sodium salt added in step d) is sodium acetate or NaClO₄, more preferably in the form of a solution in a solvent system comprising one or more polar protic solvents, preferably alcohol or water or mixture thereof, more preferably alcohol, even more preferably methanol.

Preferably the guadecitabine triethylamine salt in step a) is dissolved in a solvent system comprising one or more polar protic solvents or mixture thereof, such as alcohol and/or water. Preferably the solvent system comprises a mixture of alcohol and water, preferably ethanol or methanol and water, most preferably methanol and water. The amount of water in the solvent system is NMT about 50% v/v, NMT about 20% v/v, NMT about 10% v/v, or NMT about 5% v/v, preferably wherein the amount of water is NMT about 40% v/v, more preferably NMT about 30% v/v, and most preferably from about 10% v/v to about 30% v/v.

Preferably the antisolvent added in step e) is polar solvent. Examples of suitable aprotic antisolvents that may be used include, but are not limited to, ketones, nitriles and ethers. Preferred are AcN, acetone, ethylmethylketone, dioxane, THF. Examples of suitable protic antisolvents that may be used include alcohols, such as ethanol, isopropanol, n-propanol and n-butanol, preferably wherein the alcohols used as antisolvents are higher alcohols than the alcohols used as solvents.

In a particular embodiment, guadecitabine triethylamine salt is provided in step a) in a solvent system comprising ethanol and water wherein the amount of water is NMT about 50% v/v, NMT about 20% v/v, NMT about 10% v/v, or NMT about 5% v/v, preferably wherein the amount of water is NMT about 40% v/v, more preferably NMT about 30% v/v, and most preferably from about 10% v/v to about 30% v/v, in step b) guanidine carbonate is used, in step d) guadecitabine guanidinium salt is provided in a solvent system comprising methanol and water wherein the amount of water is NMT about 50% v/v, NMT about 20% v/v, NMT about 10% v/v, or NMT about 5% v/v, preferably wherein the amount of water is NMT about 30% v/v, more preferably NMT about 20% v/v, even more preferably NMT about 10% v/v, and most preferably from about 5% v/v to about 10% v/v and NaClO₄ is used, and in step e) ethanol is used as antisolvent.

In another particular embodiment, guadecitabine triethylamine salt is provided in step a) in a solvent system comprising methanol and water wherein the amount of water is NMT about 50% v/v, NMT about 20% v/v, NMT about 10% v/v, or NMT about 5% v/v, preferably wherein the amount of water is NMT about 40% v/v, more preferably NMT about 30% v/v, and most preferably from about 10% v/v to about 30% v/v, in step b) guanidine carbonate is used, in step d) guadecitabine guanidinium salt is provided in a solvent system comprising methanol and water wherein the amount of water is NMT about 50% v/v, NMT about 20% v/v, NMT about 10% v/v, or NMT about 5% v/v, preferably wherein the amount of water is NMT about 30% v/v, more preferably NMT about 20% v/v, even more preferably NMT about 10% v/v, and most preferably from about 5% v/v to about 10% v/v and sodium acetate is used, and in step e) methanol is used as antisolvent.

In another aspect the disclosure provides a process for preparation of guadecitabine guanidinium salt comprising steps a), b) and c) as described above.

In another aspect the disclosure provides a process for preparation of guadecitabine sodium, preferably crystalline guadecitabine sodium wherein the process comprises:
a) providing guadecitabine triethylamine salt optionally in the form of solution;
b) adding a sodium salt, optionally in the form of solution;
c) adding antisolvent; and
d) optionally isolating guadecitabine sodium.

The process may include additional washing and drying steps.

Organic or inorganic sodium salts may be used in step b). Examples of suitable salts include, but are not limited to, acetate, formate, propionate, nitrate and perchlorate salts. Preferably the sodium salt added is step b) is NaClO₄, more preferably in the form of a solution in a solvent system comprising a polar protic solvent, preferably alcohol or water, more preferably alcohol, even more preferably methanol.

Preferably the guadecitabine triethylamine salt in step a) is dissolved in a solvent system comprising one or more polar protic solvents, such as alcohol and/or water, with the proviso that water is not to be used as a single solvent. Preferably the solvent system comprises a mixture of alcohol and water, preferably methanol and water, preferably the amount of water in the solvent system is NMT about 50% v/v, NMT about 20% v/v, NMT about 10% v/v, or NMT about 5% v/v, preferably wherein the amount of water is NMT about 40% v/v, more preferably NMT about 30% v/v, and most preferably from about 10% v/v to about 30% v/v.

Preferably the antisolvent added in step c) is a polar solvent. Examples of suitable aprotic solvents that may be used include, but are not limited to, ketones, nitriles and ethers. Preferred are AcN, acetone, ethylmethylketone, dioxane, THF. Examples of suitable protic solvents that may be used include alcohols, such as ethanol, isopropanol, n-propanol and n-butanol wherein the alcohols used as antisolvents are higher alcohols than the alcohols used as solvents.

In a particular embodiment, guadecitabine triethylamine salt is provided in step a) in a solvent system comprising methanol and water preferably wherein the amount of water is NMT 50% v/v, in step b) NaClO₄ is used and in step c) acetone is used as antisolvent.

In one embodiment, the present disclosure provides a process for preparation of guadecitabine or salts thereof comprising:
a) oxidation of compound 10a

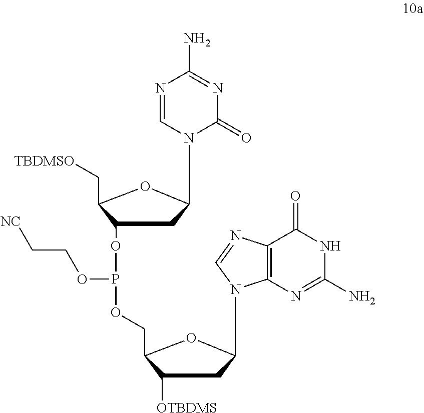 [SMILES: CC(C)(C)[Si](C)(C)OC[C@H]1O[C@@H](n2cnc(N)nc2=O)C[C@H]1OP(OCCC#N)OC[C@H]1O[C@@H](n2cnc3c(=O)[nH]c(N)nc32)C[C@H]1O[Si](C)(C)C(C)(C)C]

to afford compound 11a

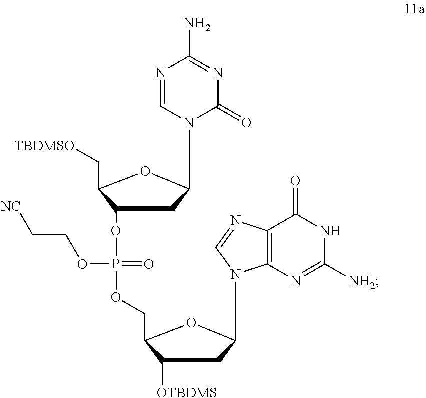 [SMILES: CC(C)(C)[Si](C)(C)OC[C@H]1O[C@@H](n2cnc(N)nc2=O)C[C@H]1OP(=O)(OCCC#N)OC[C@H]1O[C@@H](n2cnc3c(=O)[nH]c(N)nc32)C[C@H]1O[Si](C)(C)C(C)(C)C]

b) conversion of compound 11a to guadecitabine (compound 1) or salts thereof.

Step a) is typically carried out in the presence of a suitable solvent, and a suitable oxidation reagent. Suitable solvents may include, for example, dimethylformamide (DMF), dimethylacetamide (DMA), N-methyl-2-pyrrolidone (NMP), dimethylsulphoxide (DMSO), acetonitrile, toluene, methyl tert-butyl ether (MTBE), n-heptane, n-hexane, dichloromethane (DCM), n-nonane, 1,4-dioxane or mixtures thereof. Preferably, the solvent is 1,4-dioxane, DMF, acetonitrile or combination thereof. More preferably, the solvent is a combination of acetonitrile and DMF. In another embodiment, the reaction is performed in DMF.

Suitable oxidation reagents include but are not limited to iodine, peroxides, peroxoacids, hydroperoxides, halogens or mixtures thereof. Preferably iodine and hydroperoxide, most preferably t-butyl hydroperoxide (t-BuOOH) are used.

Preferably the reaction is performed in anhydrous conditions.

Conversion of compound 11a to guadecitabine or salts thereof typically includes deprotection of the hydroxy groups and cleavage of the cyanoethyl group, i.e. deprotection of phosphate group. The hydroxyl groups deprotection may be performed before or after the phosphate deprotection, or all protecting groups can be cleaved concurrently.

In a particular embodiment, step b) comprises the following steps:
i) deprotecting compound 11a;
ii) forming a basic salt of guadecitabine and optionally isolating guadecitabine or a salt thereof the salt; and
iii) optionally converting the basic salt formed in step ii) to guadecitabine or to another basic salt thereof.

Step i) is typically carried out in the presence of a suitable solvent, a suitable base and a fluoride reagent. Suitable solvents may include, for example, DMA (dimethylacetamide), DMF, acetonitrile, NMP, DMSO, toluene, DCM, MeOH, EtOH, i-PrOH, THF, n-hexane, n-heptane or mixture thereof. Preferably, the solvent is DMA (dimethylacetamide), DMF, DMSO, acetonitrile, toluene, DCM, THF or combination thereof. More preferably, the solvent is acetonitrile or DMA (dimethylacetamide). In a particular embodiment the solvent system used is a mixture of DMA and DMSO. The ratio of DMA:DMSO may be about, 4:1, preferably about 6:1 to about 10:1, more preferably about 20:1 to about 30:1 v/v.

Suitable bases include but are not limited to Et₃N, diisopropylethylamine, DABCO, pyridine, DBU, DBN, K₂CO₃ and other amines and other carbonates. Preferably Et3N, diisopropylethylamine may be used, most preferably triethylamine is used.

Suitable fluoride reagent include but are not limited to pyridine hydrogen fluoride (Py.HF), Tetrabutylammonium fluoride (TBAF), tris(dimethylamino)sulfonium difluorotrimethylsilicate (TASF), Et3N*3HF, CsF. Preferably may be used TBAF and Et₃N*3HF. Most preferably Et₃N*3HF is used. Other salts of organic base and hydrogenfluoride may be used too.

Preferably in step ii) a triethylamine salt of guadecitabine is formed.

In a specific embodiment, guadecitabine triethylamine salt may be converted to guadecitabine or to another salt of guadecitabine in step iii) such as guadecitabine guanidinium or guadecitabine Na, preferably guadecitabine Na.

In a specific embodiment, guadecitabine Na formed may be crystalline, preferably it may be crystalline form A or crystalline form B.

In a specific embodiment guadecitabine guanidinium formed may be crystalline, preferably it may be crystalline form G1, or form G2.

In another particular embodiment, step b) comprises the following steps:
i) deprotecting compound 11a;
ii) formation of a triethylamine salt of guadecitabine and optionally isolating the salt;
iii) optionally converting the triethylamine salt of guadecitabine formed in step v) to a guanidinium salt of guadecitabine; and
iv) optionally converting the guadecitabine guanidium formed in step iii) to guadecitabine or another basic salt thereof, preferably guadecitabine Na.

Step i) is typically carried out in the presence of a suitable solvent, a suitable base and a fluoride reagent. Suitable solvents may include, for example, DMA (dimethylacetamide), DMF, DMSO, acetonitrile (AcN), NMP, toluene, DCM, MeOH, EtOH, i-PrOH, THF, n-hexane, n-heptane or mixture thereof. Preferably, the solvent is DMA (dimethylacetamide), DMF, DMSO, acetonitrile, toluene, DCM, tetrahydrofuran (THF) or combination thereof. More preferably, the solvent is acetonitrile or DMA (dimethylacetamide) In a particular embodiment the solvent system used is a mixture of DMA and DMSO. The ratio of DMA:DMSO may be about 4:1, preferably about 6:1 to about 10:1, more preferably about 20:1 to about 30:1 v/v.

Suitable bases include but are not limited to Et₃N, diisopropylethylamine, 1,4-diazabicyclo[2.2.2]octane (DABCO), pyridine, 1,8-diazabicyclo[5.4.0]undec-7-ene (DBU), 1,5-diazabicyclo[4.3.0]non-5-ene (DBN), K₂CO₃ and other amines and other carbonates. Preferably Et₃N or diisopropylethylamine may be used, most preferably triethylamine is used.

Suitable fluoride reagent include but are not limited to pyridine hydrogen fluoride (Py.HF), Tetrabutylammonium fluoride (TBAF), tris(dimethylamino)sulfonium difluorotrimethylsilicate (TASF), Et3N*3HF, CsF. Other salts of organic base and hydrogenfluoride may be used too.

Preferably may be used TBAF and Et3N*3HF. Most preferably Et3N*3HF is used.

In a specific embodiment, guadecitabine guanidinium formed in step iii) may be crystalline, preferably it may be crystalline form G1, or form G2.

In a specific embodiment guadecitabine Na formed in step iv) may be crystalline, preferably it may be crystalline form A or crystalline form B.

In a particularly preferred embodiment, the disclosure provides a process for preparation of guadecitabine Na comprising:
a) oxidizing of compound 10, preferably compound 10a to afford compound 11, preferably 11a;
b) deprotecting compound 11 or preferably 11a in the presence of a suitable base, a fluoride reagent and a solvent wherein the solvent comprises DMA and DMSO, the base is triethylamine to afford a triethylammonium salt of guadecitabine;
c) providing the triethylammonium salt of guadecitabine in a mixture of an alcohol and water, preferably methanol and water, more preferably wherein the amount of water is NMT about 50% v/v, NMT about 20% v/v, NMT about 10% v/v, or NMT about 5% v/v, preferably wherein the amount of water is NMT about 40% v/v, more preferably NMT about 30% v/v, and most preferably from about 10% v/v to about 30% v/v;
d) adding a guanidine salt, preferably guanidine carbonate, more preferably a solution of guanidine carbonate in water and optionally adding an antisolvent to afford guadecitabine guanidinium, preferably in crystalline form;
e) providing the guadecitabine guanidinium in a mixture of an alcohol and water, preferably methanol and water, more preferably wherein the wherein the amount of water is NMT about 50% v/v, NMT about 20% v/v, NMT about 10% v/v, or NMT about 5% v/v, preferably wherein the amount of water in the solvent system is NMT about 30% v/v, more preferably NMT about 20% v/v, even more preferably NMT about 10% v/v, and most preferably from about 5% v/v to about 10% v/v; and
f) adding a sodium salt, preferably sodium acetate, more preferably a solution of sodium acetate in methanol and optionally adding an antisolvent to afford guadecitabine Na, preferably in crystalline form.

In another embodiment the disclosure comprises processes for preparation of guadecitabine sodium comprising:
a) providing guadecitabine guanidinium in a mixture of an alcohol and water, preferably methanol and water, more preferably wherein the amount of water in the solvent system is NMT about 50% v/v, NMT about 20% v/v, NMT about 10% v/v, or NMT about 5% v/v, preferably wherein the amount of water is NMT about 30% v/v, more preferably NMT about 20% v/v, even more preferably NMT about 10% v/v, and most preferably from about 5% v/v to about 10% v/v;
b) adding a sodium salt, preferably sodium acetate, more preferably a solution of sodium acetate in methanol and optionally adding an antisolvent to afford guadecitabine Na, preferably in crystalline form;
   any of the above processes may include steps of heating, cooling, washing and drying.

In another embodiment the disclosure comprises processes for preparation of guadecitabine sodium comprising:
a) Providing guadecitabine triethylammonium salt in a mixture of methanol and water, wherein the amount of water is NMT about 50% v/v, NMT about 20% v/v, NMT about 10% v/v, or NMT about 5% v/v, preferably wherein the amount of water is NMT about 40% v/v, more preferably NMT about 30% v/v, and most preferably from about 10% v/v to about 30% v/v;
b) Optionally heating the reaction mixture to a temperature of from about 30° C. to about 50° C.;
c) Adding guanidine carbonate, preferably in the form of a solution in water
d) Optionally cooling the reaction mixture to a temperature of from about 0° C. to about 25° C.;
e) Optionally adding antisolvent;
f) Optionally isolating guadecitabine guanidinium;
g) Providing guadecitabine guanidinium salt in a mixture of methanol and water wherein the amount of water is NMT about 50% v/v, NMT about 20% v/v, NMT about 10% v/v, or NMT about 5% v/v, preferably wherein the amount of water is NMT about 30% v/v, more preferably NMT about 20% v/v, even more preferably NMT about 10% v/v, and most preferably from about 5% v/v to about 10% v/v;
h) Optionally heating the reaction mixture to a temperature of about 30° C. to about 50° C.;
i) Adding sodium acetate, preferably in the form of a solution in methanol;
j) Optionally cooling the reaction mixture to a temperature of from about 0° C. to about 25° C.;
k) Optionally adding antisolvent;
l) Separating guadecitabine sodium;
m) Optionally washing or drying.

In another embodiment, the present disclosure relates to processes for preparation of guadecitabine as described above wherein compound 10 is prepared by a process comprising:
A) coupling compound A

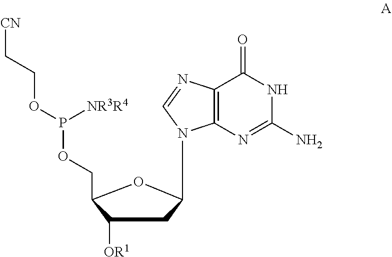

and compound 7

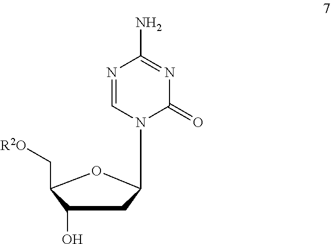

wherein R¹, R², R³ and R⁴ are as defined above;
or
B) coupling compound 4

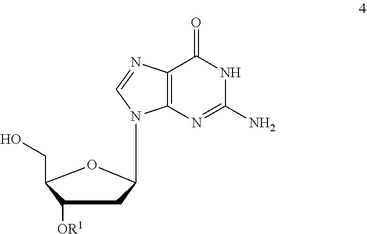

and compound B

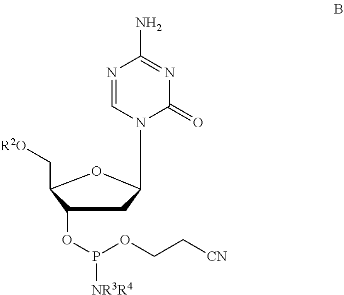

wherein R¹, R², R³ and R⁴ are as defined above.

Preferably, compound 10 is prepared by a process comprising:
A) coupling compound 5

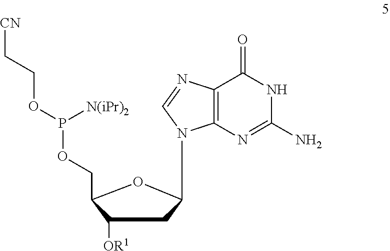

and

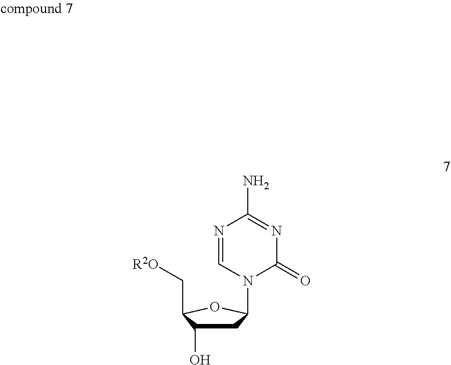

wherein R¹ and R² are as defined above; or
B) coupling compound 4

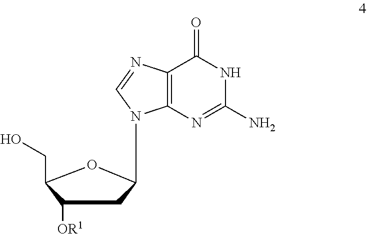

and compound 9

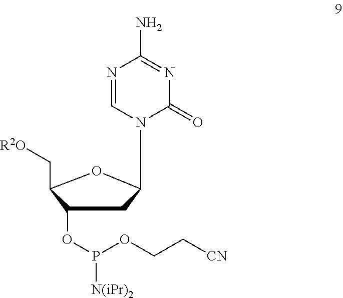

wherein R¹ and R² are as defined above.

In another particular embodiment, the present disclosure relates to processes for preparation of guadecitabine as described above wherein compound 10a is prepared by a process comprising:
A) coupling compound 5a

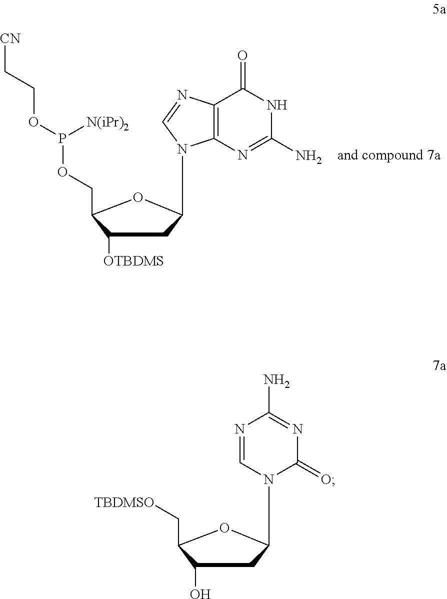

or
B) coupling compound 4a

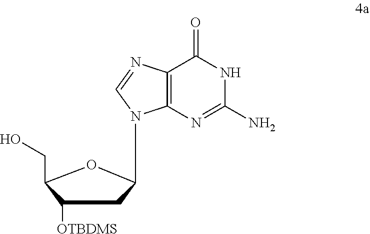 [SMILES: CC(C)(C)[Si](C)(C)O[C@@H]1C[C@H](n2cnc3c(=O)[nH]c(N)nc32)O[C@@H]1CO]

and compound 9a

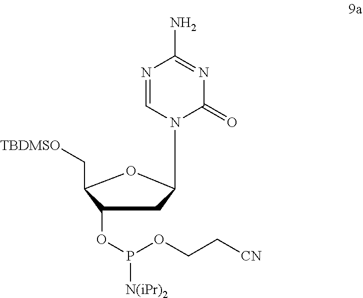

Steps A) and B) are typically carried out in the presence of a suitable solvent, a suitable activator and optionally a base. Suitable solvents may include aprotic polar solvents, for example, acetonitrile, DMF, DMA, THF, NMP, 1,4-dioxane, dimethylsulfoxide (DMSO), toluene or mixture thereof. Preferably, the solvent is DMF, acetonitrile, 1,4-dioxane or combination thereof. More preferably, the solvent is a combination of acetonitrile and DMF. In another preferred embodiment the solvent comprises DMF, preferably the solvent is a mixture of DMF and toluene.

Suitable activators include but are not limited to anhydrous acidic compounds that include but are not limited to Lewis acids, acidic heterocycles for example optionally substituted 1H-tetrazole, substituted triazole including but not limited to 1H-tetrazole, 5-(ethylthio)-1H-tetrazole, 5-(benzylthio)-1H-tetrazole, substituted imidazole, including but not limited to 4,5-dicyanoimidazole, 2-bromo-4,5-dicyanoimidazole, optionally substituted 1-hydroxy-benzotriazol, salts of organic bases, such as salts of amines or nitrogen-containing heterocyclic compounds, for example ammonium, pyridinium and azolium including but not limited to pyridinium trifluoroacetate (Py-TFA), imidazolium tetrafluoroborate, imidazolium triflate, imidazoliumtrifluoroacetate, N-methylimidazolium tetrafluoroborate, N-methylimidazolium triflate, N-methylimidazolium trifluoroacetate, N-phenylimidazolium tetrafluoroborate, N-phenylimidazolium triflate or N-phenylimidazolium trifluoroacetate, benzimidazolium tetrafluoroborate, benzimidazolium triflate or benzimidazolium trifluoroacetate, N-methylbenzimidazolium tetrafluoroborate, N-methylbenzimidazolium triflate or N-methylbenzimidazolium trifluoroacetate, organic acids, for example such as trifluoroacetic acid, saccharin, dichloroacetic acid, trichloroacetic acid, trifluoroacetic acid, inorganic acid including but not limited to HClO₄, HCl, HBr, HI, HBF₄, HPF₆, methanesulfonic acid and trifluoromethanesulfonic acid, and 2,4-dinitrophenol. Preferably the activator is a salt of a nitrogen containing heterocycle such as pyridinium trifluoroacetate (Py-TFA), imidazolium tetrafluoroborate, imidazolium triflate, imidazolium trifluoroacetate, N-methylimidazolium tetrafluoroborate, N-methylimidazolium triflate, N-methylimidazolium trifluoroacetate, benzimidazolium tetrafluoroborate, benzimidazolium triflate, benzimidazolium trifluoroacetate, N-methylbenzimidazolium tetrafluoroborate, N-methylbenzimidazoliumtriflate or N-methylbenzimidazolium trifluoroacetate. More preferably, the activator is pyridinium trifluoroacetate (Py-TFA). In another preferred embodiment the activator is N-methylimidazolium trifluoroacetate.

Suitable bases include but are not limited to N-methylimidazole (NMI), imidazole, N-phenylimidazole, benzimidazole, N-methylbenzimidazole. Preferably the base is N-methylimidazole (NMI), imidazole. More preferably the base is N-methylimidazole (NMI).

In one embodiment compound B

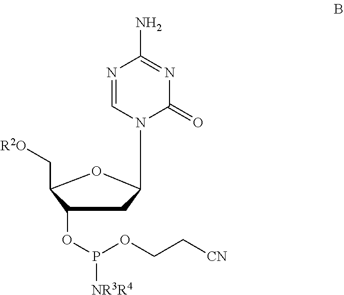

may be prepared by reacting compound 7

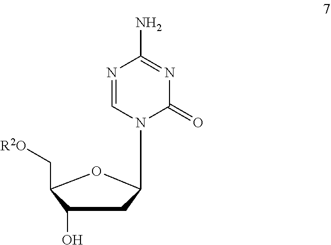

with compound C:

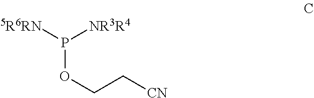

wherein R¹ and R² are as described above, R³, R, R⁵ and R⁶ may be the same or different and are selected from the group consisting of hydrogen, methyl, ethyl, n-propyl, isopropyl, trimethylsilyl, or R³ and R⁴ or R⁵ and R⁶ taken together with the intervening nitrogen atom form an optionally substituted heterocyclyl such as a pyrrolidine, piperidine, morpholine, 2,2,6,6-tetramethylpiperidine, imidazole, 4,5-dichloroimidazole, triazole, or tetrazole. Preferably R³, R⁴, R⁵ and R⁶ are isopropyl.

Compound C may be prepared in situ from compound D:

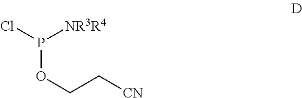

wherein R³ and R⁴ are the same as for compound C above; in the presence of a suitable amine. Suitable amines include but are not limited to optionally substituted imidazole, 4,5-dichloroimidazole, triazole and tetrazole.

In a preferred embodiment compound 9

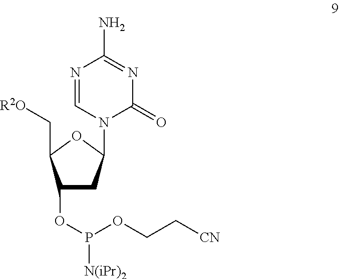

may be produced by reacting compound 7

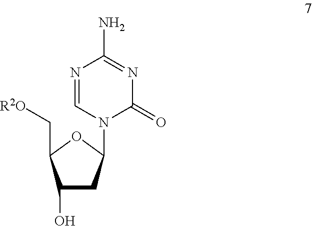

and cyanoethyltetraisopropyl phosphorodiamidite (compound 8).

Ina particular embodiment, compound 9a

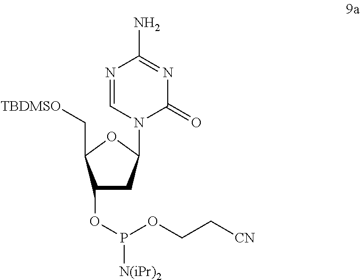

may be produced by reacting compound 7a

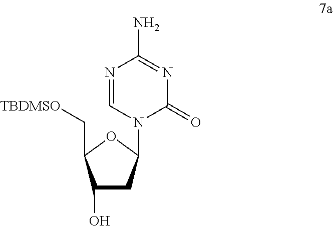 [SMILES: CC(C)(C)[Si](C)(C)OC[C@H]1O[C@@H](n2cnc(N)nc2=O)C[C@H]1O]

and cyanoethyltetraisopropyl phosphorodiamidite (compound 8).

The reaction may typically be carried out in the presence of a suitable solvent, and a suitable activator. Suitable solvents may include, for example, acetonitrile, DMF, DMA, NMP, DCM, toluene, MTBE, 1,4-dioxane, THF or mixture thereof. More preferably, the solvent is acetonitrile, DMF or mixtures thereof. Most preferably the solvent is DMF.

Suitable activators include but are not limited to anhydrous acidic compounds that include but are not limited to Lewis acids, acidic heterocycles for example optionally substituted 1H-tetrazole, substituted triazole including but not limited to 1H-tetrazole, 5-(ethylthio)-1H-tetrazole, 5-(benzylthio)-1H-tetrazole, substituted imidazole, including but not limited to 4,5-dicyanoimidazole, 2-bromo-4,5-dicyanoimidazole, optionally substituted 1-hydroxy-benzotriazol, salts of organic bases, such as salts of amines or nitrogen-containing heterocyclic compounds, for example ammonium, pyridinium and azolium including but not limited to pyridinium trifluoroacetate (Py-TFA), imidazolium tetrafluoroborate, imidazolium triflate, imidazoliumtrifluoroacetate, N-methylimidazolium tetrafluoroborate N-methylimidazolium triflate, N-methylimidazolium trifluoroacetate, N-phenylimidazolium tetrafluoroborate, N-phenylimidazolium triflate or N-phenylimidazolium trifluoroacetate, benzimidazolium tetrafluoroborate, benzimidazolium triflate or benzimidazolium trifluoroacetate, N-methylbenzimidazolium tetrafluoroborate, N-methylbenzimidazolium triflate or N-methylbenzimidazolium trifluoroacetate, organic acids, for example such as trifluoroacetic acid, saccharin, dichloroacetic acid, trichloroacetic acid, trifluoroacetic acid, inorganic acid including but not limited to HClO₄, HCl, HBr, HI, HBF₄, HPF₆, methanesulfonic acid and trifluoromethanesulfonic acid and 2,4-dinitrophenol. Preferably the activator is a salt of a nitrogen containing heterocycle such as pyridinium trifluoroacetate (Py-TFA), imidazolium tetrafluoroborate, imidazolium triflate, imidazolium trifluoroacetate, N-methylimidazolium tetrafluoroborate, N-methylimidazolium triflate, N-methylimidazolium trifluoroacetate, benzimidazolium tetrafluoroborate, benzimidazolium triflate, benzimidazolium trifluoroacetate, N-methylbenzimidazolium tetrafluoroborate, N-methylbenzimidazoliumtriflate or N-methylbenzimidazolium trifluoroacetate. More preferably, the activator is pyridinium trifluoroacetate (Py-TFA). In another preferred embodiment the activator is N-methylimidazolium trifluoroacetate.

In one embodiment compound A

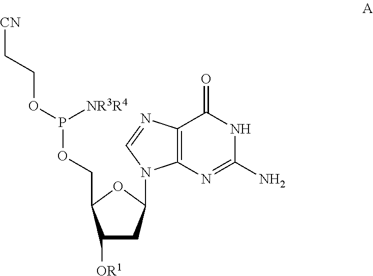

may be prepared by reacting compound 4

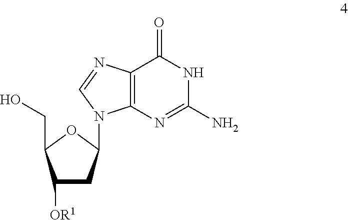

with compound C:

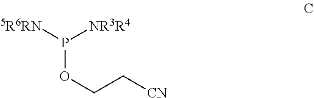

wherein R³, R⁴, R⁵ and R⁶ may be the same or different and are selected from the group consisting of hydrogen, methyl, ethyl, n-propyl, isopropyl, trimethylsilyl, or R3 and R⁴ or R⁵ and R⁶ taken together with the intervening nitrogen atom form an optionally substituted heterocyclyl such as a pyrrolidine, piperidine, morpholine, 2,2,6,6-tetramethylpiperidine, imidazole, 4,5-dichloroimidazole, triazole, or tetrazole. Preferably R³, R⁴, R⁵ and R⁶ are isopropyl.

Compound C may be prepared in situ from compound D:

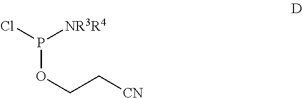

wherein R³ and R⁴ are the same as for compound C above; in the presence of a suitable amine. Suitable amines include but are not limited to optionally substituted imidazole, 4,5-dichloroimidazole, triazole and tetrazole.

In another embodiment compound 5

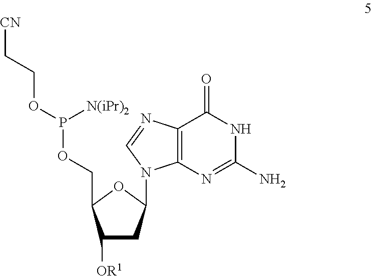

may be produced by reacting compound 4

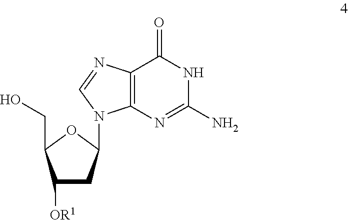

and cyanoethyltetraisopropyl phosphorodiamidite (compound 8).

In a particular embodiment compound 5a

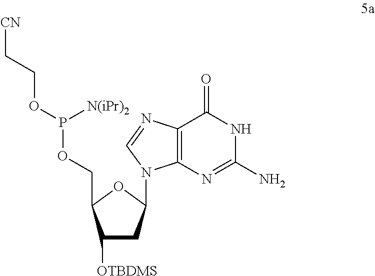

may be produced by reacting compound 4a

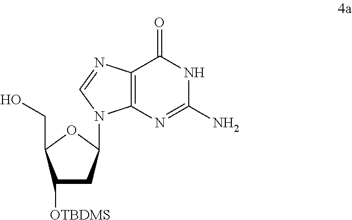 [SMILES: CC(C)(C)[Si](C)(C)O[C@@H]1C[C@H](n2cnc3c(=O)[nH]c(N)nc32)O[C@@H]1CO]

and cyanoethyltetraisopropyl phosphorodiamidite (compound 8).

The reaction may typically be carried out in the presence of a suitable solvent, and a suitable activator. Suitable solvents may include, for example, acetonitrile, DMF, DMA, NMP, DCM, toluene, MTBE, 1,4-dioxane, THF or mixture thereof. More preferably, the solvent is acetonitrile, DMF or mixtures thereof. Most preferably the solvent is DMF.

Suitable activators include but are not limited to anhydrous acidic compounds that include but are not limited to Lewis acids, acidic heterocycles for example optionally substituted 1H-tetrazole, substituted triazole including but not limited to 1H-tetrazole, 5-(ethylthio)-1H-tetrazole, 5-(benzylthio)-1H-tetrazole, substituted imidazole, including but not limited to 4,5-dicyanoimidazole, 2-bromo-4,5-dicyanoimidazole, optionally substituted 1-hydroxy-benzotriazol, salts of organic bases, such as salts of amines or nitrogen-containing heterocyclic compounds, for example ammonium, pyridinium and azolium including but not limited to pyridinium trifluoroacetate (Py-TFA), imidazolium tetrafluoroborate, imidazolium triflate, imidazoliumtrifluoroacetate, N-methylimidazolium tetrafluoroborate N-methylimidazolium triflate, N-methylimidazolium trifluoroacetate, N-phenylimidazolium tetrafluoroborate, N-phenylimidazolium triflate or N-phenylimidazolium trifluoroacetate, benzimidazolium tetrafluoroborate, benzimidazolium triflate or benzimidazolium trifluoroacetate, N-methylbenzimidazolium tetrafluoroborate, N-methylbenzimidazolium triflate or N-methylbenzimidazolium trifluoroacetate, organic acids, for example such as trifluoroacetic acid, saccharin, dichloroacetic acid, trichloroacetic acid, trifluoroacetic acid, inorganic acid including but not limited to HClO₄, HCl, HBr, HI, HBF₄, HPF₆, methanesulfonic acid and trifluoromethanesulfonic acid and 2,4-dinitrophenol. Preferably the activator is a salt of a nitrogen containing heterocycle such as pyridinium trifluoroacetate (Py-TFA), imidazolium tetrafluoroborate, imidazolium triflate, imidazolium trifluoroacetate, N-methylimidazolium tetrafluoroborate, N-methylimidazolium triflate, N-methylimidazolium trifluoroacetate, benzimidazolium tetrafluoroborate, benzimidazolium triflate, benzimidazolium trifluoroacetate, N-methylbenzimidazolium tetrafluoroborate, N-methylbenzimidazoliumtriflate or N-methylbenzimidazolium trifluoroacetate.

More preferably, the activator is pyridinium trifluoroacetate (Py-TFA). In another preferred embodiment the activator is N-methylimidazolium trifluoroacetate

In a further embodiment comp und 7

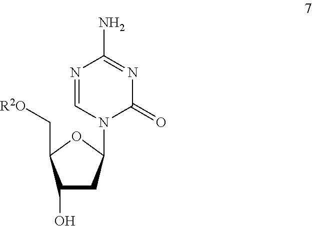

may be produced by a process comprising reacting compound 6

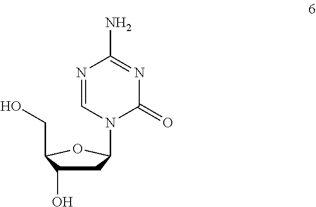

with an appropriate protecting agent.

Specifically, compound 7a

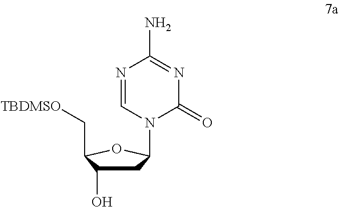

may be produced in a selective mono-silylation reaction comprising reacting compound 6 with t-Butyldimethylsilyl chloride.

The selective mono-silylation reaction may be carried out in the presence of a suitable solvent, and a silyl protecting agent in combination with suitable base. The reaction may typically be carried out in the presence of a suitable solvent, and a suitable activator. Suitable solvents may include, for example, acetonitrile, DMF, NMP, N,N′-dimethylimidazolidone (DMI), N,N′-dimethylpropyleneurea (DMPU), N-methylimidazole, dimethylacetamide (DMA), DMSO, DCM, toluene, MTBE, 1,4-dioxane, THF or mixture thereof. Preferably, the solvent is DMF, DMA and acetonitrile or combination thereof. Most preferably, the solvent is DMA.

In a particular embodiment compound 7a may be produced by a process comprising: providing decitabine in a polar aprotic solvent in the presence of a base; cooling to a temperature of about −15° C. to about 5° C., about −5° C. to about 5° C.; adding the silylating agent and optionally isolating compound 7a.

The first step may include heating to afford dissolution of decitabine.

Suitable polar aprotic solvents include but are not limited to acetonitrile, DMF, NMP, N,N′-dimethylimidazolidone (DMI), N,N′-dimethylpropyleneurea (DMPU), N-methylimidazole, dimethylacetamide (DMA), DMSO or mixtures thereof. Preferably the solvent is DMF.

Suitable bases include but are not limited to imidazole, N-methylimidazole, pyridine, dimethylaminopyridine (DMAP), tertiary amines as triethylamine and diisopropylethylamine. Preferably the base is imidazole.

Suitable silyl protecting agents include but are not limited to trimethylsilyl chloride, hexamethyldisilazane, triethylsilyl chloride, triisopropylsilyl chloride, tert-butyldimethylsilyl chloride, tert-butyldiphenylsilyl chloride. Preferably triethylsilyl chloride, triisopropylsilyl chloride, tert-butyldimethylsilyl chloride, tert-butyldiphenylsilyl chloride. More preferably tert-butyldimethylsilyl chloride.

The process can be illustrated by the following Scheme 4:

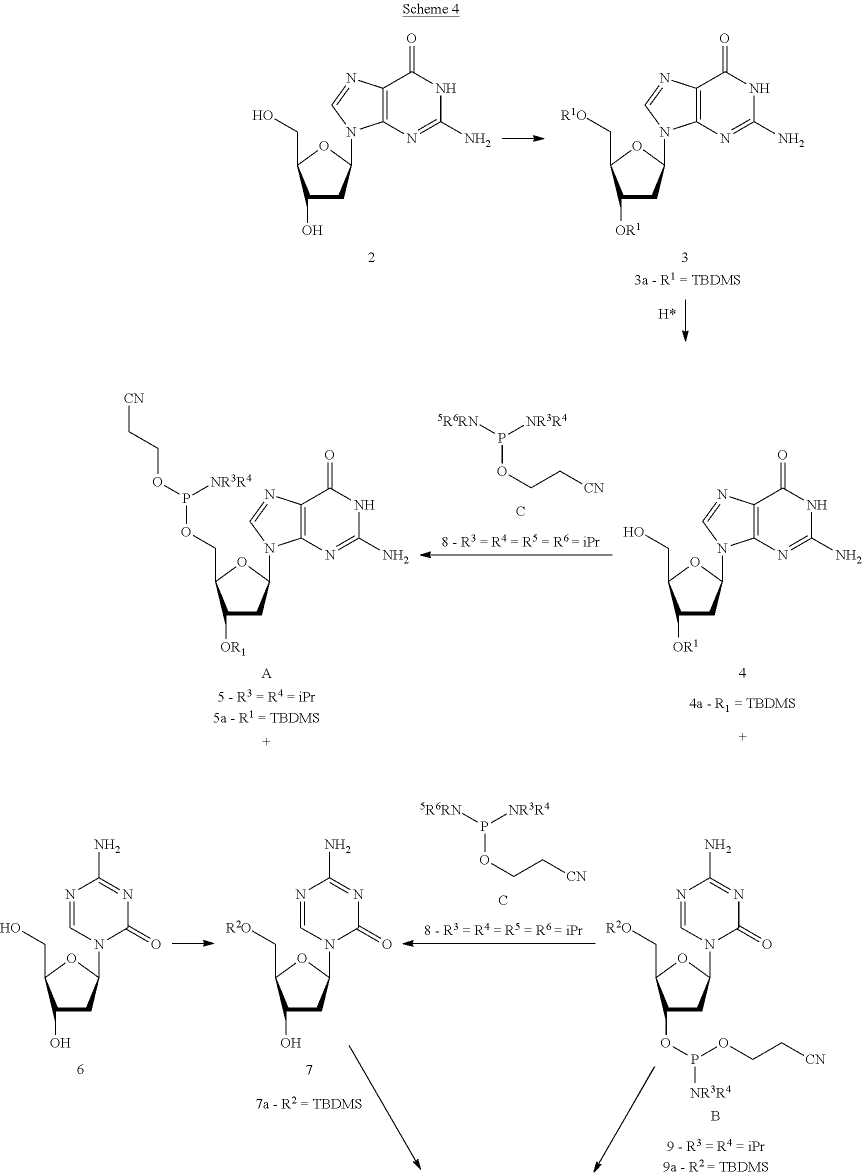

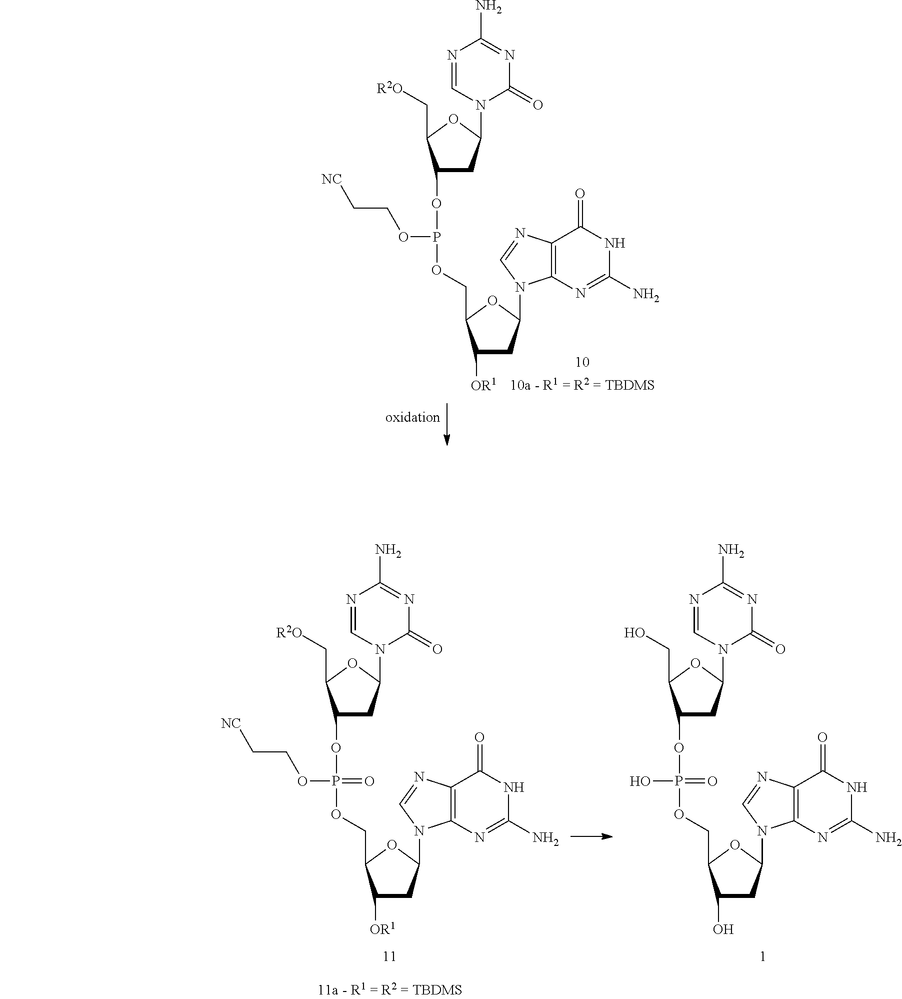

In another embodiment, the disclosure provides processes for preparation of compound 10

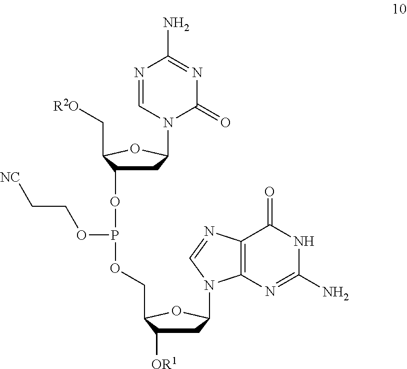

comprising:
A) coupling compound 5

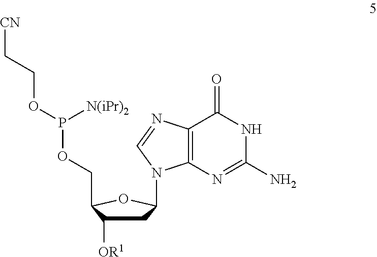

and compound 7

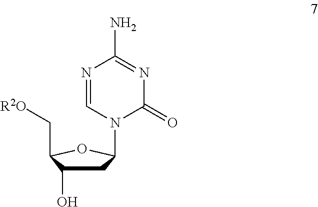

wherein R¹ and R² are as described above;
or
B) coupling compound 4

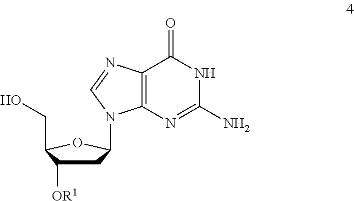

and compound 9

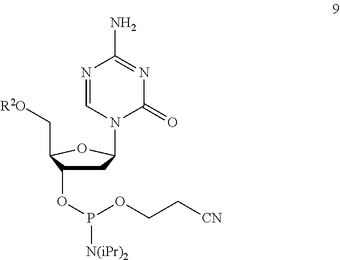

wherein R¹ and R² are as described above. Steps A and B may be performed as described above.

In another embodiment the above process further comprises converting compound 10 to guadecitabine.

In another embodiment, the disclosure provides processes for preparation of compound 10a

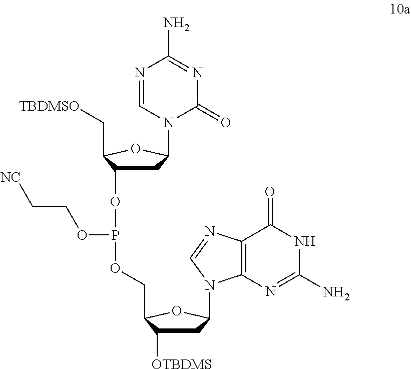

comprising:
A) coupling compound 5a

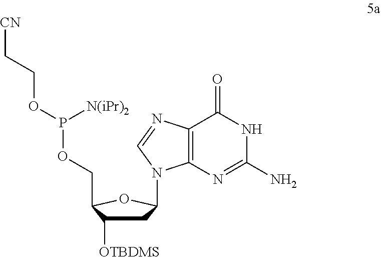

and compound 7a

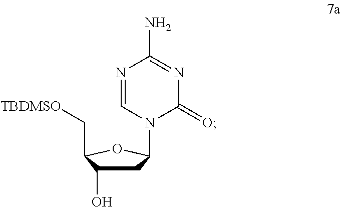

or
B) coupling compound 4a

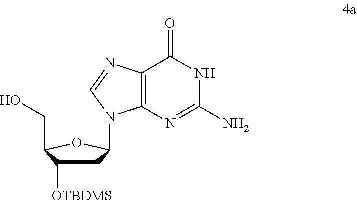

and compound 9a

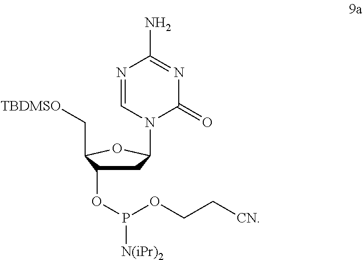

Steps A and B may be performed as described above.

In another embodiment the above process further comprises converting compound 10a to guadecitabine.

In another embodiment, the disclosure provides a process for preparation of compound 11

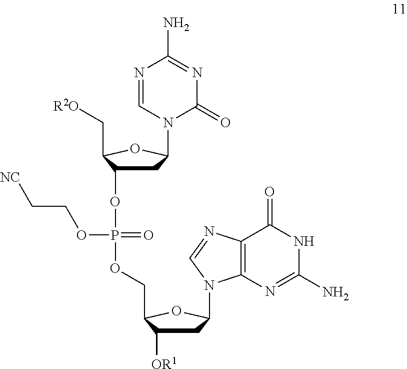

comprising oxidation of compound 10.

In a specific embodiment, the disclosure provides a process for preparation of compound 11a

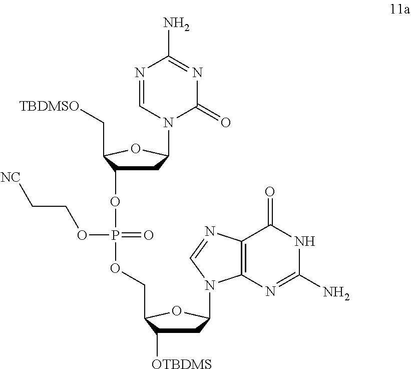

comprising oxidation of compound 10a.

In another embodiment the disclosure further provides a process for preparation of compound 9

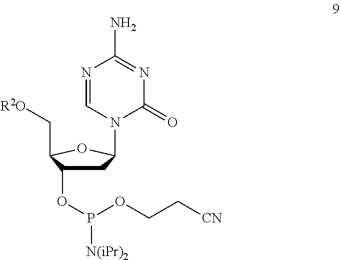

comprising reacting compound 7

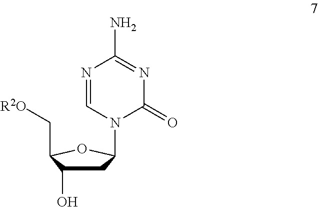

wherein R¹ and R² are as described above;
with cyanoethyltetraisopropyl phosphorodiamidite (compound 8).

In a specific embodiment the disclosure further provides a process for preparation of compound 9a

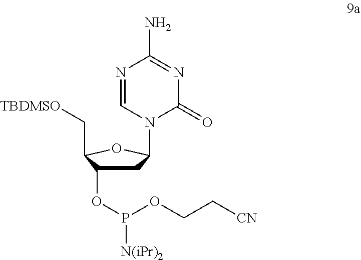

comprising reacting compound 7a

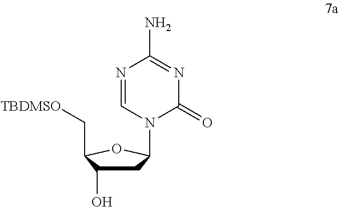

with cyanoethyltetraisopropyl phosphorodiamidite (compound 8).

In another embodiment the disclosure further provides a process for preparation of compound 5

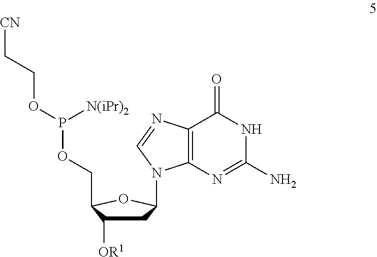

comprising reacting compound 4

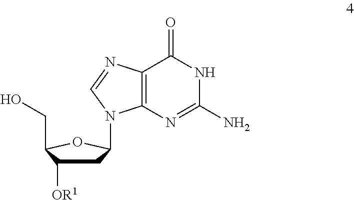

with cyanoethyltetraisopropyl phosphorodiamidite (compound 8).

In a specific embodiment the disclosure further provides a process for preparation of compound 5a

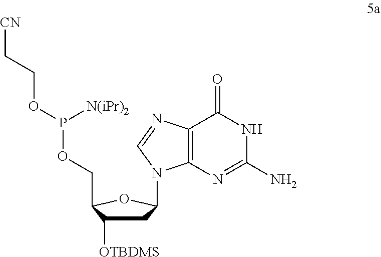

comprising reacting compound 4a

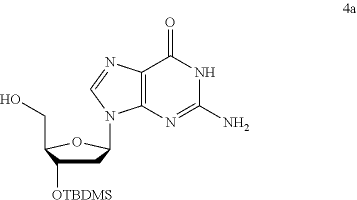

with cyanoethyltetraisopropyl phosphorodiamidite (compound 8).

In another embodiment the disclosure further provides a process for preparation of compound 7

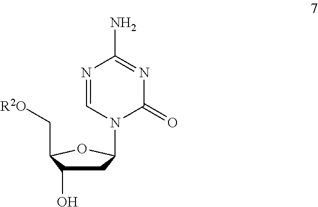

comprising protecting compound 6.

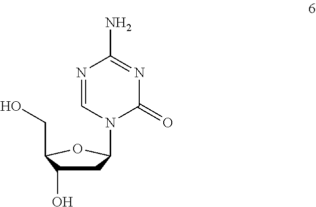

In a specific embodiment the disclosure further provides a process for preparation of compound 7a

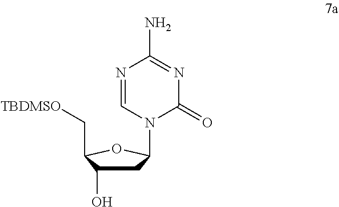

comprising protecting compound 6.

In another aspect, the present disclosure provides any one of compounds 5, 7, 9, 10 and 11 produced by the process of the present disclosure.

In one embodiment, the present disclosure provides any one of compounds 5a, 7a, 9a, 10a and 11a produced by the process of the present disclosure.

In another aspect the present disclosure provides Guadecitabine produced by the processes of the present disclosure.

In another embodiment the disclosure further provides processes for preparation of guadecitabine or salts thereof, preferably guadecitabine Na, comprising:
a) Reacting compound 4, preferably wherein R1 is TBDMS (compound 4a) with compound B, preferably wherein R3 and R4 are isopropyl (compound 9), more preferably wherein R3 and R4 are isopropyl and R2 is TBDMS (compound 9a) to provide compound 10, preferably wherein R1 and R2 are TBDMS (compound 10a);
b) Oxidizing compound 10, preferably wherein R1 and R2 are TBDMS (compound 10a); to afford compound 11, preferably wherein R1 and R2 are TBDMS (compound 11a); and
c) Converting the compound produced in step b) to guadecitabine or salt thereof, preferably guadecitabine Na.

In another embodiment the disclosure further relates to the above embodiment wherein compound B is prepared by reacting compound 7 with compound C, preferably wherein compound C is prepared in situ from compound D.

In another embodiment the disclosure further relates to the above embodiment wherein compound 9 is prepared by reacting compound 7 with cyanoethyltetraisopropyl phosphoramidite (compound 8).

In another embodiment the disclosure further relates to the above embodiment wherein compound 9a is prepared by reacting compound 7a with cyanoethyltetraisopropyl phosphoramidite (compound 8).

In another embodiment the disclosure further relates to any of the above embodiments wherein compound 7 is prepared by selective mono-silylation of compound 6, preferably compound 7a is prepared by selective mono-silylation of compound 6 comprising reacting compound 6 with t-butyldimethylsilyl chloride. In another embodiment the disclosure further relates to any of the above embodiments wherein step c) comprises:
i) deprotecting compound 11 or preferably 11a in the presence of a suitable base, a fluoride reagent and a solvent wherein the solvent comprises DMA and DMSO, the base is triethylamine to afford a triethylammonium salt of guadecitabine;
ii) providing the triethylammonium salt of guadecitabine in a mixture of an alcohol and water, preferably methanol and water, more preferably wherein the amount of water is NMT about 50% v/v, NMT about 20% v/v, NMT about 10% v/v, or NMT about 5% v/v, preferably wherein the amount of water is NMT about 40% v/v, more preferably NMT about 30% v/v, and most preferably from about 10% v/v to about 30% v/v;
(iii) adding a guanidine salt, preferably guanidine carbonate, more preferably a solution of guanidine carbonate in water and optionally adding an antisolvent to afford guadecitabine guanidinium, preferably in crystalline form;
iv) providing the guadecitabine guanidinium in a mixture of an alcohol and water, preferably methanol and water, more preferably wherein the wherein the amount of water is NMT about 50% v/v, NMT about 20% v/v, NMT about 10% v/v, or NMT about 5% v/v, preferably wherein the amount of water in the solvent system is NMT about 30% v/v, more preferably NMT about 20% v/v, even more preferably NMT about 10% v/v, and most preferably from about 5% v/v to about 10% v/v; and
v) adding a sodium salt, preferably sodium acetate, and optionally adding an antisolvent to afford guadecitabine Na, preferably in crystalline form.

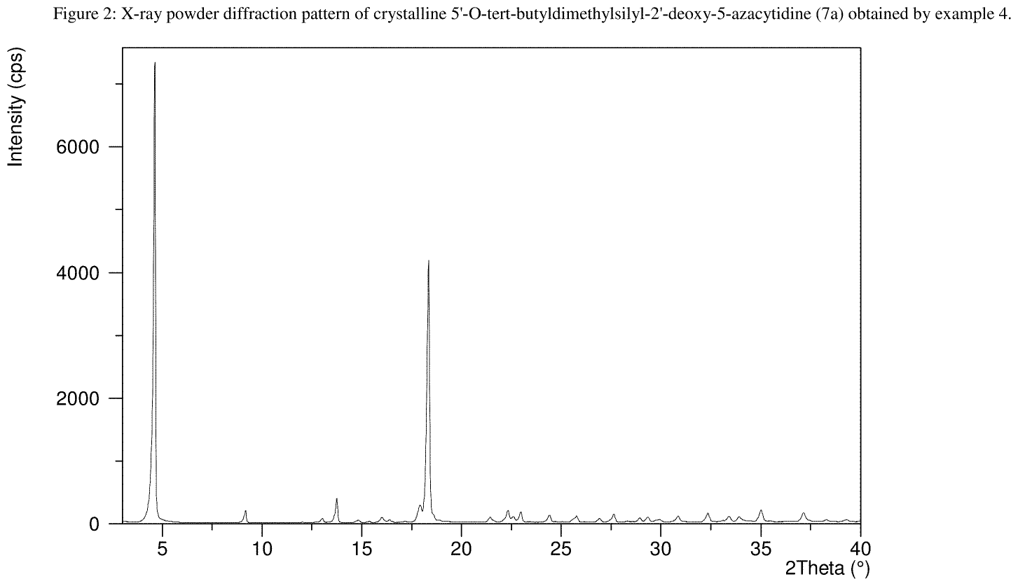

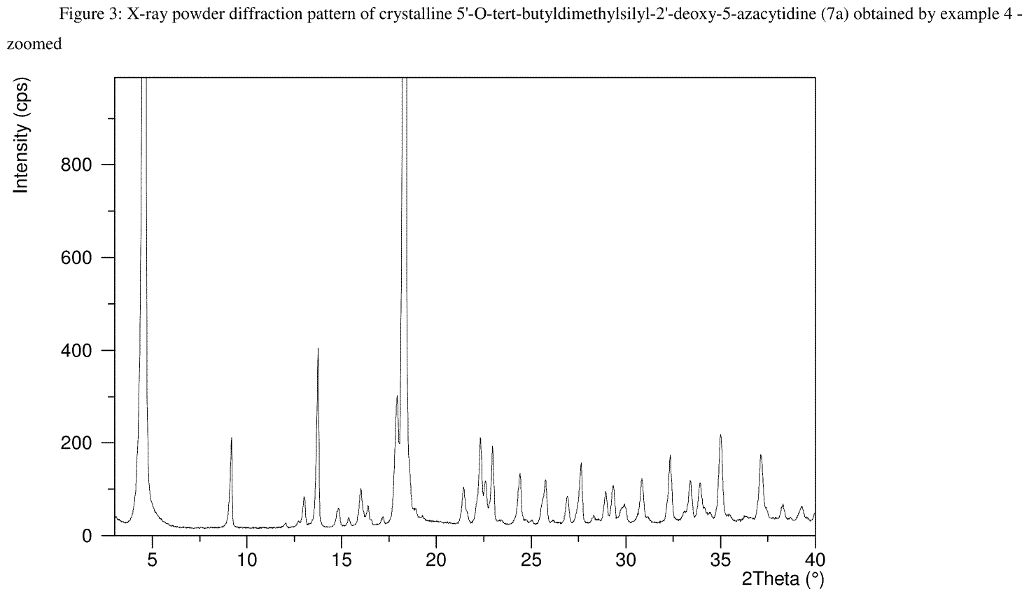

In another aspect, the present disclosure comprises a crystalline form of 5′-O-tert-butyldimethylsilyl-2′-deoxy-5-azacytidine (7a). The crystalline form of 5′-O-tert-butyldimethylsilyl-2′-deoxy-5-azacytidine (7a) can be characterized by data selected from one or more of the following: an XRPD pattern having peaks at 4.6, 9.2, 13.0, 13.8, and 18.4 degrees 2-theta±0.2 degrees 2-theta; and an XRPD pattern as depicted in FIG. 2 or FIG. 3.

The crystalline form of 5′-O-tert-butyldimethylsilyl-2′-deoxy-5-azacytidine (7a) may be further characterized by the XRPD pattern having peaks at 4.6, 9.2, 13.0, 13.8, and 18.4 degrees 2-theta±0.2 degrees 2-theta, and also having one, two, three, four or five additional peaks at 14.7, 22.3, 23.0, 24.4 or 27.6 degrees 2-theta±0.2 degrees 2-theta.

In another embodiment the present disclosure provides use of the crystalline form of 5′-O-tert-butyldimethylsilyl-2′-deoxy-5-azacytidine (7a) for the preparation of guadecitabine or a salt thereof, preferably guadecitabine triethylamine, guadecitabine guanidinium or guadecitabine sodium salts, more preferably guadecitabine Na.

The present disclosure provides the crystalline form of 5′-O-tert-butyldimethylsilyl-2′-deoxy-5 azacytidine (7a) for use in the preparation of guadecitabine or a salt thereof.

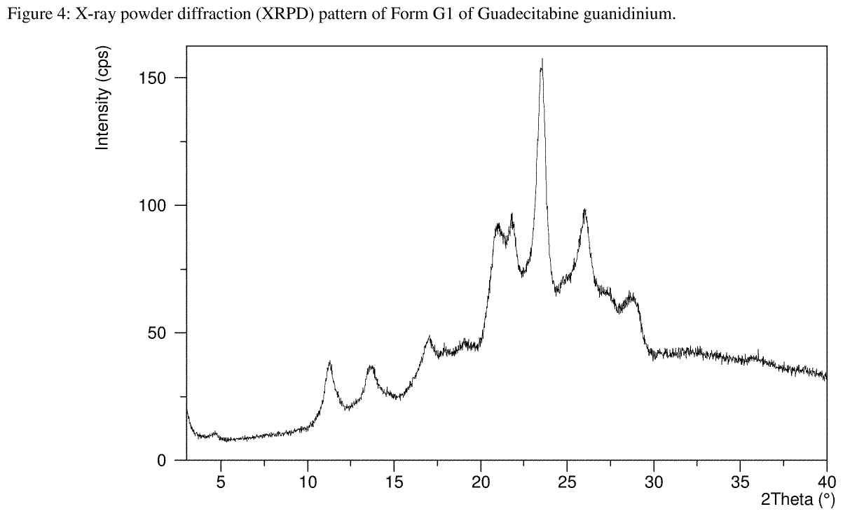

In another aspect the present disclosure comprises a crystalline Form of guadecitabine guanidinium designated form G. The crystalline Form G1 of guadecitabine guanidinium can be characterized by data selected from one or more of the following: an XRPD pattern having peaks at 11.1, 13.5, 16.9, 23.3 and 25.8 degrees 2-theta±0.2 degrees 2-theta; and an XRPD pattern substantially as depicted in FIG. 4.

Crystalline Form G1 of guadecitabine guanidinium may be further characterized by an XRPD pattern having peaks at 11.1, 13.5, 16.9, 23.3 and 25.8 degrees two theta±0.2 degrees two theta, and also having one, two, three, or four additional peaks selected from 4.5, 20.8, 21.6 and 28.6 degrees two theta±0.2 degrees two theta.

In one embodiment of the present disclosure, crystalline Form G1 of guadecitabine guanidinium is isolated in solid form.

Crystalline Form G1 of guadecitabine guanidinium may be polymorphically pure.

In some instances Form G1 may be semi crystalline.

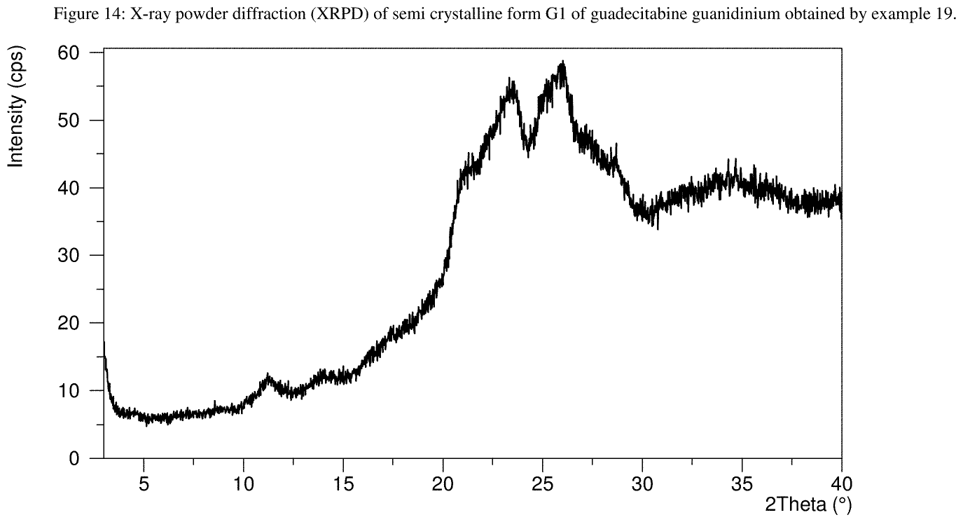

Semi crystalline form G1 may be characterized by data selected from one or more of the following: an XRPD pattern having peaks at 11.1, 23.3 and 25.8 degrees 2-theta±0.2 degrees 2-theta; and an XRPD pattern substantially as depicted in FIG. 14.

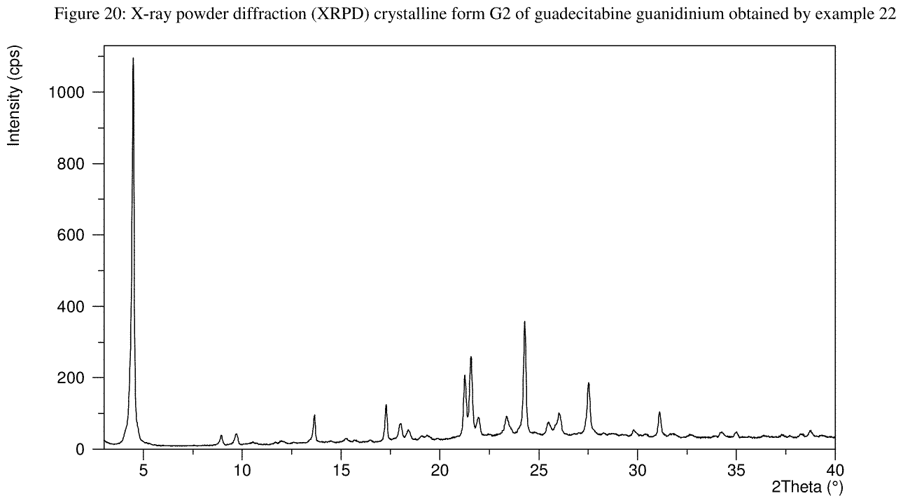

In another aspect the present disclosure comprises a crystalline Form of guadecitabine guanidinium designated form G2. The crystalline Form G2 of guadecitabine guanidinium can be characterized by data selected from one or more of the following: an XRPD pattern having peaks at 4.5, 17.3, 21.6, 24.3 and 27.5 degrees 2-theta±0.2 degrees 2-theta; and an XRPD pattern substantially as depicted in FIG. 20.

Crystalline Form G2 of guadecitabine guanidinium may be further characterized by an XRPD pattern having peaks at 4.5, 17.3, 21.6, 24.3 and 27.5 degrees two theta±0.2 degrees two theta, and also having one, two, three, four or five additional peaks selected from 8.9, 9.7, 13.6, 17.9 and 21.3 degrees two theta±0.2 degrees two theta.

In one embodiment of the present disclosure, crystalline Form G2 of guadecitabine guanidinium is isolated in solid form.

Crystalline Form G2 of guadecitabine guanidinium may be polymorphically pure.

In another embodiment the present disclosure provides use of guadecitabine guanidinium, amorphous, semicrystalline or crystalline, and preferably crystalline form G1 or form G2 for the preparation of guadecitabine or another salt thereof, preferably guadecitabine sodium.

The present disclosure provides guadecitabine guanidinium and preferably crystalline form G1 or form G2 of guadecitabine guanidinium for use in the preparation of guadecitabine or another salt thereof, preferably guadecitabine Na. In another aspect the disclosure provides crystalline guadecitabine sodium.

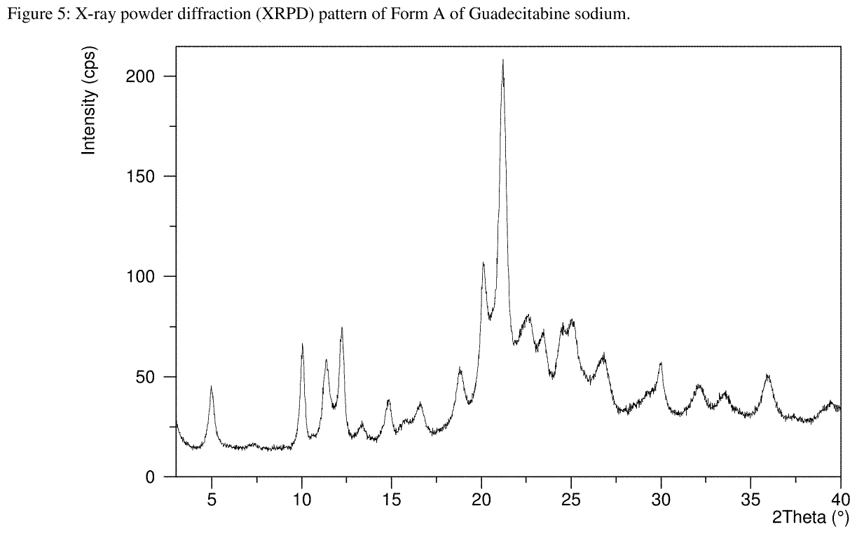

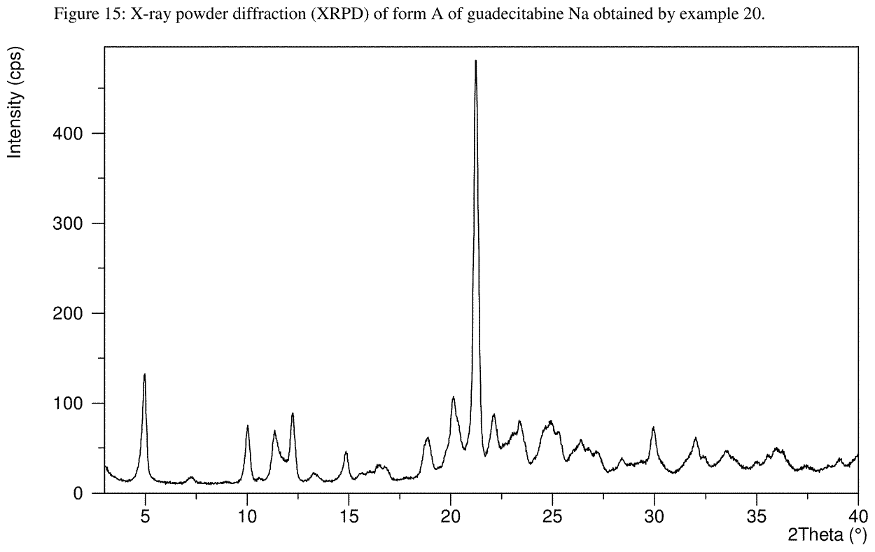

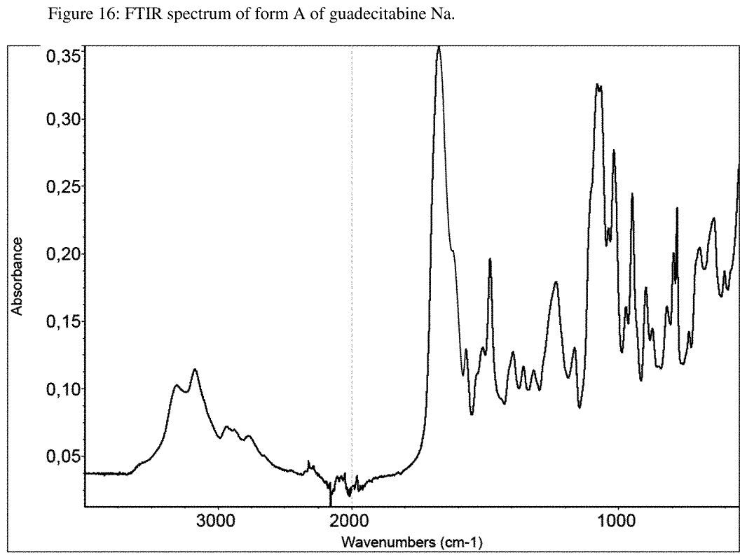

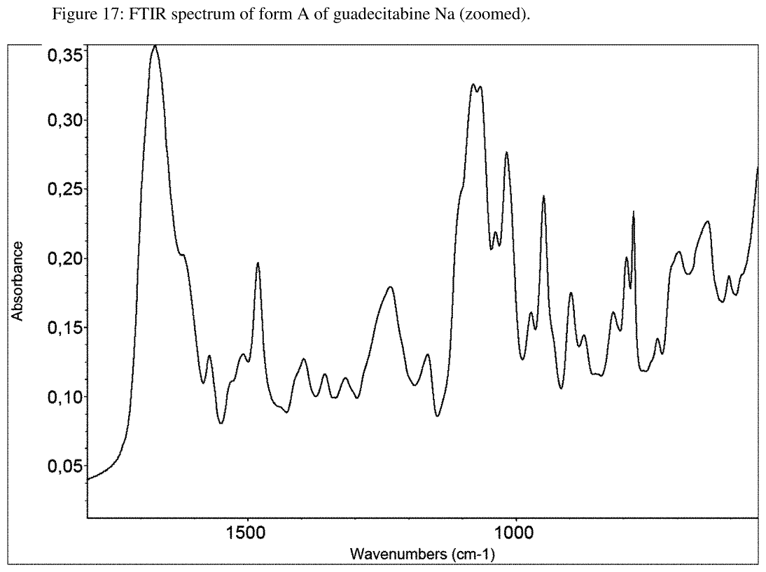

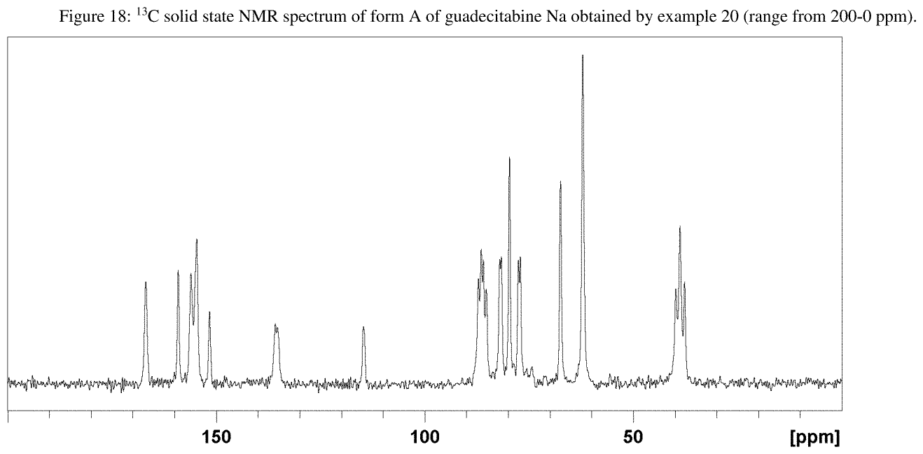

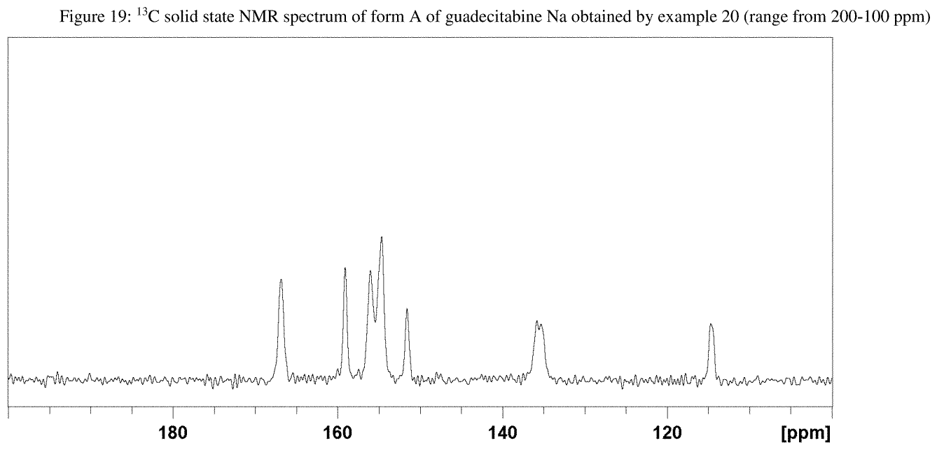

In another aspect the present disclosure comprises a crystalline Form of guadecitabine Na designated form A. The crystalline Form A of guadecitabine sodium can be characterized by data selected from one or more of the following: an XRPD pattern having peaks at 10.0, 11.3, 12.2, 20.1 and 21.0 degrees 2-theta±0.2 degrees 2-theta; an XRPD pattern substantially as depicted in FIG. 5 or in FIG. 15; a solid state ¹³C NMR spectrum substantially as depicted in FIG. 18 or in FIG. 19; a solid state ¹³C NMR spectrum having peaks at 166.9, 159.1, 156.0, 154.7 and 151.6 ppm±0.2 ppm; a solid state ¹³C NMR spectrum having the following chemical shift absolute differences between said characteristic peaks at 166.9, 159.1, 156.0, 154.7 and 151.6 ppm±0.2 ppm and a reference peak at 114.7 ppm±1 ppm of 52.2, 44.4, 41.3, 40.0 and 36.9 ppm±0.1 ppm; a solid state ¹³C NMR spectrum having peaks at 166.9, 159.1, 156.0, 154.7, 151.6, 135.8, 135.3, 114.7, 87.1, 86.4, 85.9, 85.2, 82.0, 81.6, 79.7, 77.5, 77.1, 67.4, 62.1, 39.8, 38.8 and 37.7 ppm±0.2 ppm; an FT-IR spectrum substantially as depicted in FIG. 16 or in FIG. 17; an FT-IR spectrum having absorptions at 1674, 1080, 1018, 949 and 782 cm⁻¹±4 cm⁻¹; and an FT-IR spectrum having absorptions at 3308, 3176, 2941, 1674, 1572, 1509, 1482, 1396, 1357, 1319, 1235, 1165, 1080, 1039, 1018, 973, 949, 898, 874, 819, 794, 782, 737, 697, 643 and 604 cm⁻¹±4 cm⁻¹.

Crystalline Form A of guadecitabine sodium may be further characterized by an XRPD pattern having peaks at 10.0, 11.3, 12.2, 20.1 and 21.0 degrees two theta±0.2 degrees two theta, and also having one, two, three, four or five additional peaks selected from 4.9, 14.8, 16.5, 18.7 and 29.7 degrees two theta±0.2 degrees two theta.

Crystalline form A of guadecitabine sodium may be characterized by the data set forth in the following table.

TABLE 1
X-ray powder diffraction peaks of
form A of guadecitabine sodium.
peak position
(degrees
two theta ±
0.2 degrees
two theta)

4.9
10.0
11.3
12.2
13.3
14.8
16.5
18.7
20.1
21.0
23.2
24.9
26.3
29.7
32.1
33.4
35.9

In one embodiment of the present disclosure, Form A of guadecitabine sodium is isolated in solid form.

Crystalline Form A of guadecitabine sodium is polymorphically pure.

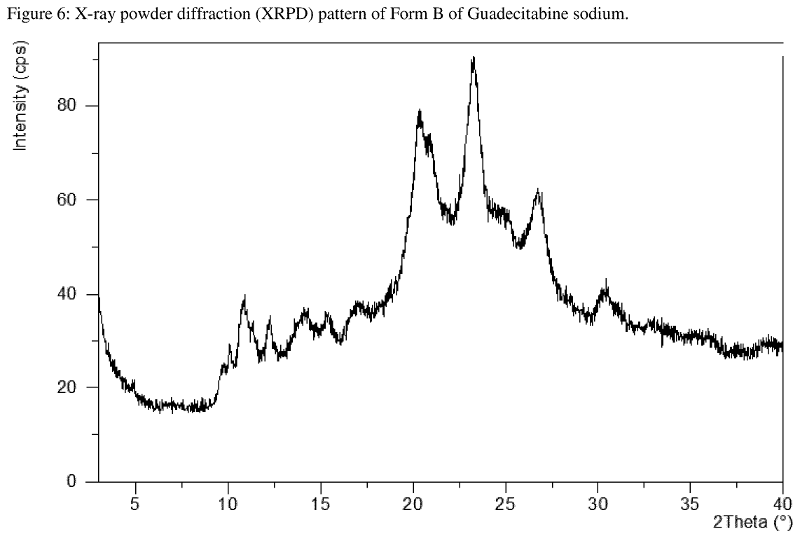

In another aspect the present disclosure comprises a crystalline Form of guadecitabine Na designated form B. The crystalline Form B of guadecitabine sodium can be characterized by data selected from one or more of the following: an XRPD pattern having peaks at 10.8, 12.2, 20.3, 23.3 and 26.7 degrees 2-theta±0.2 degrees 2-theta; and an XRPD pattern substantially as depicted in FIG. 6.

Crystalline Form B of guadecitabine sodium may be further characterized by an XRPD pattern having peaks at 10.8, 12.2, 20.3, 23.3 and 26.7 degrees two theta±0.2 degrees two theta, and also having one, two, three or four additional peaks selected from 14.1, 15.4, 16.8 and 30.3 degrees two theta±0.2 degrees two theta.

In one embodiment of the present disclosure, Form B of guadecitabine sodium is isolated in solid form.

Crystalline Form B of guadecitabine sodium may be polymorphically pure.

In another aspect the present disclosure provides guadecitabine guanidinium salt.

In another embodiment the present disclosure provides use of guadecitabine guanidinium salt, amorphous, semi-crystalline or crystalline, preferably crystalline or semi-crystalline forms G1 and G2 thereof, for preparation of guadecitabine sodium, preferably crystalline guadecitabine sodium.

In another embodiment the present disclosure provides guadecitabine guanidinium salt, amorphous, semi-crystalline or crystalline, preferably crystalline or semi-crystalline forms G1 and G2 thereof, for preparation of guadecitabine sodium, preferably crystalline guadecitabine sodium.

In another embodiment the disclosure relates crystalline guadecitabine guanidinium, amorphous, semi-crystalline or crystalline, preferably crystalline semi-crystalline forms G1 and G2 for use in the preparation of other salts of guadecitabine, preferably, crystalline, semi crystalline forms or amorphous guadecitabine sodium.

As discussed above Guadecitabine guanidinium offers significant impurity purging capabilities. Thus, it was surprisingly found that guadecitabine guanidinium or any solid form thereof produced by the processes of the present disclosure contains about 0.15 wt % or less, preferably about 0.10 wt % or less, more preferably 0.05 wt % or less of each one of degradant 2a and degradant 2b and/or contains about 0.25 wt % or less, preferably about 0.20 wt % or less, more preferably 0.15 wt % or less of each one of degradant 2c and degradant 2d, the structures of which are depicted in Scheme 3. The content of the impurities is measured by HPLC.

Moreover, it has been surprisingly found that guadecitabine guanidinium or any solid form thereof produced by the processes of the present disclosure may have a total impurity content of: about 0.9 wt % or less, about 0.5 wt % or less, about 0.3 wt % or less, preferably about 0.2 wt % or less, most preferably about 0.1 wt % or less, as measured by HPLC.

The solid form of guadecitabine guanidinium salt may be any amorphous, semi crystalline or crystalline form, preferably form G1 or G2.

In another embodiment the disclosure relates to use of crystalline guadecitabine sodium, preferably crystalline forms A or B in the preparation of other crystalline forms, semi crystalline forms or amorphous guadecitabine sodium.

In another embodiment the disclosure relates crystalline guadecitabine sodium, preferably crystalline forms A or B for use in the preparation of other crystalline forms, semi crystalline forms or amorphous guadecitabine sodium.

As discussed above Guadecitabine guanidinium offers significant impurity purging capabilities.

Further, it was surprisingly found that Guadecitabine Sodium or any solid form thereof produced by the processes of the present disclosure contains about 0.15 wt % or less, preferably about 0.10 wt % or less, more preferably 0.05 wt % or less as measured by HPLC of each one of degradant 2a and degradant 2b and/or contains about 0.25 wt % or less, preferably about wt 0.20% or less, more preferably wt 0.15% or less as measured by HPLC of each one of degradant 2c and degradant 2d, the structures of which are depicted in Scheme 3. The content of the impurities is measured by HPLC.

Moreover, it has been surprisingly found that guadecitabine sodium or any solid form thereof produced by the processes of the present disclosure may have a total impurity content of: about 0.9 wt % or less, about 0.5 wt % or less, about 0.3 wt % or less, preferably about 0.2 wt % or less, most preferably about 0.1 wt % or less, as measured by HPLC.

Guadecitabine or salts thereof, preferably guadecitabine triethylamine, guadecitabine guanidinium and respective crystalline forms thereof, produced by the processes of the present disclosure may be used to prepare salts of Guadecitabine.

Guadecitabine or guadecitabine salts preferably guadecitabine triethylamine, guadecitabine guanidinium, guadecitabine sodium and respective crystalline forms thereof, produced by the processes of the present disclosure may be used in the preparation of pharmaceutical compositions of guadecitabine or guadecitabine salts.

The present disclosure also encompasses the use of the guadecitabine or guadecitabine salts prepared by the processes of the present disclosure for the preparation of pharmaceutical compositions of guadecitabine or guadecitabine salts.

The present disclosure comprises processes for preparing the above combining the guadecitabine prepared by the processes of the present disclosure or salts thereof with at least one pharmaceutically acceptable excipient.

Guadecitabine or salts thereof prepared by the processes of the present disclosure and the pharmaceutical compositions of guadecitabine or salts thereof prepared by the processes of the present disclosure can be used as medicaments, particularly for the treatment of cancer.

The present disclosure also provides methods for the treatment of cancer, comprising administering a therapeutically effective amount of guadecitabine or salts thereof prepared by the processes of the present disclosure, or at least one of the above pharmaceutical compositions, to a subject in need of the treatment.

Pharmaceutical formulations of the present invention contain any one or a combination of the solid state forms of guadecitabine or salts thereof of the present invention, particularly crystalline guadecitabine sodium and more particularly form A of guadecitabine sodium. In addition to the active ingredient, the pharmaceutical formulations of the present invention can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate. Flavoring agents and flavor enhancers make the dosage form more palatable to the patient Common flavoring agents and flavor enhancers for pharmaceutical products that can be included in the composition of the present invention include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, guadecitabine sodium and any other solid excipients are dissolved or suspended in a liquid carrier such as vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present invention, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present invention include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the present invention is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present invention can be a capsule containing the composition, preferably a powdered or granulated solid composition of the invention, within either a hard or soft shell. The shell can be made from gelatin and optionally contain a plasticizer such as glycerin and sorbitol, and an opacifying agent or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, but they are not subjected to a final tableting step.

A pharmaceutical formulation of guadecitabine or salts thereof of the present invention, particularly crystalline guadecitabine sodium and more particularly form A of guadecitabine sodium can be administered. Guadecitabine or salts thereof of the present invention, particularly crystalline guadecitabine sodium and more particularly form A or form B of guadecitabine sodium is preferably formulated for administration to a mammal, preferably a human, by injection. Guadecitabine or salts thereof of the present invention, particularly crystalline guadecitabine sodium and more particularly form A or form B of guadecitabine sodium can be formulated, for example, as a viscous liquid solution or suspension, preferably a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

In some embodiments guadecitabine sodium may be formulated in substantially anhydrous solvents.

In another aspect of the present disclosure provides a formulation comprising: (a) crystalline guadecitabine sodium, preferably crystalline form A dissolved in (b) a substantially anhydrous solvent.

In another aspect of the present disclosure provides a formulation comprising: (a) crystalline guadecitabine sodium, preferably crystalline form A dissolved in (b) a substantially anhydrous solvent comprising about 45% to about 85% propylene glycol; about 5% to about 45% glycerin; and 0% to about 30% ethanol.

In some embodiments, the formulation further comprises dimethyl sulfoxide (DMSO), optionally at a DMSO:compound ratio of about 2:about 1; about 1:about 1; about 0.5:about 1; about 0.3:about 1; or about 0.2-about 0.3:about 1.

In some embodiments, a formulation disclosed herein is suitable for administration by subcutaneous injection.

In another aspect the invention provides kit comprising: (a) a first vessel containing crystalline guadecitabine sodium, preferably crystalline form A thereof; and (b) a second vessel containing a substantially anhydrous solvent comprising about 60% to about 70% propylene glycol; about 20% to about 30% glycerin; and about 5% to about 15% ethanol (w/w/w).

In particular embodiments, crystalline guadecitabine sodium of the above disclosures may be prepared by the processes of the present disclosure.

Having described the disclosure with reference to certain preferred embodiments, other embodiments will become apparent to one skilled in the art from consideration of the specification. The disclosure is further defined by reference to the following examples describing in detail the preparation of the composition and methods of use of the disclosure. It will be apparent to those skilled in the art that many modifications, both to materials and methods, may be practiced without departing from the scope of the disclosure.

### XRPD Method

Powder X-ray Diffraction was performed on PANalytical X'Pert Pro X-Ray powder diffractometer; CuKα radiation (λ=1.541874 L); X'Celerator detector; laboratory temperature 25±2° C.; zero background sample holders. Prior to analysis, the samples were gently ground using a mortar and pestle to obtain a fine powder. The ground sample was adjusted into a cavity of the sample holder and the surface of the sample was smoothed using a cover glass.

Measurement Parameters:

| Scan range | 3-40 degrees 2-theta | |
|---|---|---|
| Scan mode | continuous | |
| Step size | 0.0167 | degrees |
| Step size | 42 | s |
| Sample spin | 60 | rpm |

| | |
|---|---|
| Sample holder | zero background silicon plate |

### Fourier Transform Infrared Spectroscopy (“FTIR”) Method:

### Equipment: Nicolet 380 FTIR Spectrometer

Mode: ATR (diamond);
Spectral range: 4000-550 cm-1;
Sample/bckg gain: 8.0;
Number of scans: 128;
Resolution: 4.0 cm-1

### ¹³C CP/MAS NMR Method:

The ¹³C CP/MAS NMR spectrum was measured at 125 MHz using Bruker Avance 500 WB/US NMR spectrometer (Karlsruhe, Germany, 2003) at magic angle spinning (MAS) frequency ωᵣ/2π=11 kHz. In all cases finely powdered samples were placed into the 4 mm ZrO2 rotors and standard CPMAS pulse program was used. During acquisition of the data a high-power dipolar decoupling TPPM (two-pulse phase-modulated) was applied. The number of scans was 1020, repetition delay was 4 s. Taking into account frictional heating of the samples during fast rotation all NMR experiments were performed at 293 K (precise temperature calibration was performed).

The ¹³C scale was calibrated with glycine as external standard (176.03 ppm—low-field carbonyl signal). The NMR spectrometer was completely calibrated and all experimental parameters were carefully optimized prior the investigation. Magic angle was set using KBr during standard optimization procedure and homogeneity of magnetic field was optimized using adamantane sample (resulting line-width at half-height Δν_{1/2} was less than 3.5 Hz at 250 ms of acquisition time).

### Analytical HPLC Method Used for Measuring Guadecitabine Purity

### Column: 150×3.0 mm Luna Omega Polar C18, 1.6 μm at 40° C.

Solvent A: 0.15 mL/l formic acid in water, pH adjusted with ammonia to 3.6.
Solvent B: acetonitrile
Gradient program:

| Time (min) | % A | % B |
|---|---|---|
| 0 | 100 | 0 |
| 21 | 96.4 | 3.6 |
| 26 | 85 | 15 |
| 36 | 10 | 90 |
| 36.1 | 100 | 0 |
| 40 | 100 | 0 |

| Gradient start | at injection | |
|---|---|---|
| Flow-rate | 0.7 | mL/min |
| Detection | UV 249 | nm |
| Column temperature | 40 | ° C. |
| Autosampler temperature | 20 | ° C. |
| Sample concentration | 10 | mg/ml |
| Diluent | DMSO | |
| Injection volume | 0.4 | μL |
| Retention time of the main peak ca. | 15 | min. |

### EXAMPLES

### Example 1: Preparation of 3′,5′-bis-O-(tert-butyldimethylsilyl)-2′-deoxyguanosine (3a)

2-Deoxyguanosine monohydrate (30 g, 101.9 mmol) was suspended in DMF (150 ml). Then toluene (150 ml) was added and water was removed by azeotropic distillation (60-80° C., reduced pressure, water content 0.07%). The suspension was cooled to 47° C. then imidazole (16.8 g, 246.6 mmol) and subsequently t-butyldimethylsilyl chloride (TBDMSCl, 35.5 g, 232.5 mmol) was added in one portion and equipment was washed with DMF (30 ml). Temperature of the mixture spontaneously increased up to 48° C. (suspension was dissolved). The mixture was stirred at 50° C. (after 10 min precipitation started) for 4 h (conversion above 98%). The mixture was cooled to RT then water (180 ml) was added. T ick suspension was stirred for 30 min and filtered. Solids were washed with water (100 ml) and dried by the stream of nitrogen. Yield: 51.85 g (93%). Crude product was used in the next step without any purification.

### Example 2: Preparation of 3′-tert-butyldimethylsilyl-2′-deoxyguanosine (4a)

3a (17 g, 34.29 mmol) was suspended in THF (140 mL) and cooled to (−5-0) C. A solution of trichloroacetic acid (35 g, 0.214 mmol) in water (35 mL) was added and stirred under nitrogen at (−5-0) ° C. for 4 h. The mixture was diluted with water (100 mL) and neutralized with sat. aq. NaHCO₃ under cooling in ice/water bath to pH=7. The suspension was cooled to 0° C. and filtered after 1 h, washed with water (100 ml) and dried under vacuum at 40° C. to yield 10.5 g (81%) off-white powder.

### Example 3: Preparation of 3′-tert-butyldimethylsilyl-2′-deoxyguanosine-5′-O-[(2-cyanoethyl)-N,N-diisopropylphosphoramidite] (5a)

4a (40 mg, 0.105 mmol) was dissolved in dry acetonitrile:dimethyl formamide mixture (2:1, 1.35 mL), then cyanoethyltetraisopropyl phosphorodiamidite (38 mg, 0.126 mmol) was added under stirring followed by addition of solid pyridinium trifluoroacetate (Py.TFA, 8 mg, 41 μmol) in one portion. The mixture was stirred overnight and crude reaction mixture was used in next step.

### Example 4: Preparation of 5′-O-tert-butyldimethylsilyl-2′-deoxy-5-azacytidine (7a)

Decitabine (100.0 g; 438 mmol) and imidazole (74.6 g; 1.095 mol) were suspended in dry DMF (1000 ml) under N₂ atm. The mixture was heated to 50° C. until suspension was dissolved then the solution was cooled to 0° C. t-Butyldimethylsilyl chloride (TBDSCl, 74.9 g; 482 mmol) was added portionwise during 30 min, so that the temperature of the reaction mixture did not exceed 5° C. Then the reaction mixture was stirred at 0° C. for 2 h. Water (3 l) was added. The formed suspension was stirred for 20 min and filtered. Solids were washed with water (2×1 l) and dried in vacuum oven (40° C., 50 mBar, 24 h). Yield 133.7 g (89%).

The obtained product was dissolved in i-PrOH (1.3 l) at 65° C. then n-hexane (1 l) was added during 30 min. The formed suspension was cooled to 0° C. Next n-hexane (900 ml) was added and suspension was stirred for 2 h at 0° C. Product was filtered, washed with n-hexane (500 ml) and dried in vacuum oven (40° C., 50 mBar, 12 h). Yield 83.5 g (56%).

The obtained product (30 g) was dissolved in boiling i-PrOH (195 ml) then n-hexane (300 ml) was added during 30 min. The formed suspension was cooled to 20° C. and stirred for next 1 h. Product was filtered, washed by n-hexane (100 ml) and dried in vacuum oven (40° C., 50 mBar, 12 h). Yield 26.8 g (89%). The obtained product was analyzed by XRPD and the pattern is shown in FIGS. 2 and 3.

### Example 5: Preparation of 5′-O-tert-butyldimethylsilyl-2′-deoxy-5-azacytidine-3′-O-[(2-cyanoethyl)-N,N-diisopropylphosphoramidite] (9a)

7a (3 g, 8.8 mmol) dried by addition of dry acetonitrile (2×50 mL) and its evaporation on RVO, then suspended in acetonitrile (90 mL). Pyridinium trifluoroacetate (Py.TFA, 1.02 g, 5.3 mmol) was added in one portion, followed by addition of cyanoethyltetraisopropyl phosphorodiamidite (3.06 mL, 9.6 mmol) in one portion. The mixture was stirred at RT for 3 h, filtered, evaporated and suspended in degassed mixture of diisopropylether/n-heptane 9:1 (20 mL). The 1st crop of product was filtered off, yield 1.6 g. Mother liquor was evaporated and treated again with DIPE/n-heptane 9:1 (10 ml) to yield 1.5 g of the 2nd crop. Similarly 3rd was isolated in yield 1.6 g. Sum of crops 3.9 g (82%). Crude product was used directly in next step.

### Example 6: Preparation of 5′-O-butyldimethylsilyl-P(O)-(2-cyanoethyl)-P-deoxo-2′-deoxy-5-azacytidylyl-(3′→5′)-3′-O-tert-butyldimethylsilyl-2′-deoxy-guanosine (10a) and 5′-O-tert-butyldimethylsilyl-P-(2-cyanoethyl)-2′-deoxy-5-azacytidylyl-(3′→5′)-3′-O-tea-butyldimethylsilyl-2′-deoxy-guanosine (11)

4a (1.01 g, 2.65 mmol) was co-evaporated with 1,4-dioxane (2×25 mL) then 9a (1.6 g, 2.95 mmol) and pyridinium trifluoroacetate (Py.TFA, 0.85 g, 4.4 mmol) were added. Mixture was suspended in dry mixture AcN/DMF 20:1 v/v (21 mL), then N-methylimidazole (0.18 mL, 2.26 mmol) was added into the mixture. Suspension was stirred for 3 h for completion. Formed 10a was directly oxidized by addition of tert-butylhydrogenperoxide (tBuOOH, 5.5 M in nonane) (0.54 mL, 2.97 mmol). The mixture was stirred for 1 h and filtered through pad of celite. The filtrate was evaporated to yield 3.24 g of crude 11a.

### Example 7: Preparation of Guadecitabine Triethylammonium Salt

Compound 11a (1.83 g, 2.18 mmol) was suspended in anhydrous acetonitrile (27 mL) then Et3N (3.65 mL, 16 mmol) was added. Commercially available salt Et₃N.3HF (2.13 mL, 13.07 mmol) was added and the mixture was gently heated to 50° C. under nitrogen atmosphere for 24 h. The suspension was cooled to RT and filtered off. Solids were washed by acetonitrile (5 ml), by toluene (5 ml), by MTBE (5 ml), by n-heptane (5 ml) and dried under reduced pressure to yield 1.2 g of crude guadecitabine triethylammonium salt.

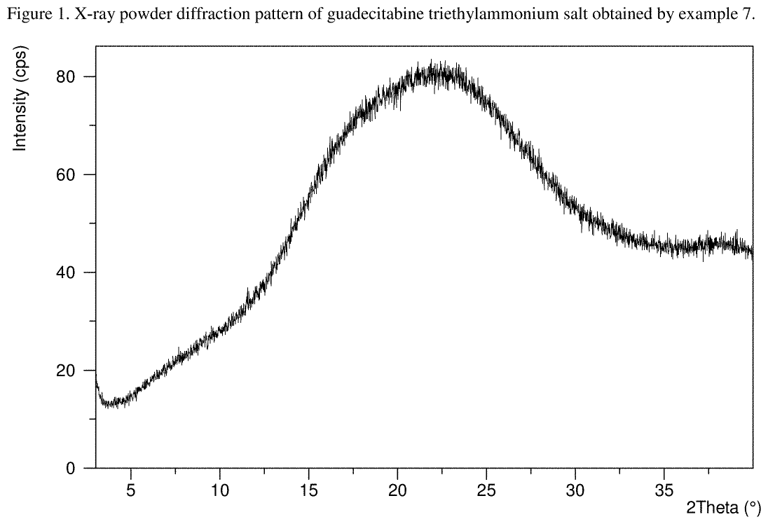

The obtained product was analyzed by XRPD and the pattern is shown in FIG. 1.

### Example 8: HPLC Purification of Guadecitabine Triethylammonium Salt

Chromatography on silica derived by aminopropyl group—BIOSPHER-PSI 100 NH, 5 um, metal column: 17×250 mm, mobile phase—isocratic: 90% MeOH+10% buffer, 300 mM TEA-Ac, pH=7.0, room temperature, UV-detector: 265 nm, flow: 20 ml/min, pressure: 130 bar, length of method: 50 minutes, effective time for repeated injections 40 minutes, injection of crude Guadecitabine.TEA: 110-150 mg in 1 ml DMSO, recommended filtration of solution=0.45 um, main fraction: 120-130 ml, 60-80 mg.

Isolation of Guadecitabine.TEA from main fraction: evaporation of MeOH+aq. buffer on RVO with portionwise addition of 1-butanol (1.5-2 vol. eq. to main fraction) divided in 4 same portions, so that volume did not fall under 100% of original volume before addition of all 1-butanol, bath temperature: 50° C., vacuum: from 100 to 8 mbar. Collection of evaporated residue as suspension in MeOH and subsequent evaporation.

### Example 9: Preparation of Form G1 of Guadecitabine Guanidinium Salt

In glovebox in 1000 ml round bottom flask equipped with magnetic stirrer, guadecitabine triethylammonium salt prepared according to the procedure of example 7 (7.80 g; 11.84 mmol) was dissolved in 90% aq. EtOH (585 ml) at 55-60° C. during 10 min. Then solution of guanidine carbonate (1.07 g, 5.94 mmol) in water (3 ml) was added. The formed suspension was stirred and cooled to 3° C. during 2 h and filtered on sintered glass filter. Solids were washed by anhydrous EtOH (50 ml) and dried by flow of nitrogen in glovebox (12 hours). Yield was 5.26 g/72%. The solid was analyzed by XRPD and the XRPD pattern is presented in FIG. 4.

### Example 10: Preparation of Form A of Guadecitabine Sodium

In glovebox in 500 ml round bottom flask equipped with magnetic stirrer, guadecitabine triethylammonium salt prepared according to the procedure of example 7 and purified according to the method of example 8 (5.53 g, 8.397 mmol) was dissolved in 90% aq. MeOH (280 ml) at 47° C., then the solution was clarified by filtration. Solution of NaClO₄ hydrate (23.59 g, 167.93 mmol) in MeOH (40 ml) was slowly added during 10 min into the stirred solution of guadecitabine triethylammonium salt. Formed suspension was stirred and cooled to 25° C. during 1 I. Acetone (250 ml) was slowly added into stirred mixture during 1 h. Suspension was stirred and cooled to 5° C. during 1 h and filtered on sintered glass filter. Precipitate was thoroughly washed by acetone (2×200 ml) and dried by flow of nitrogen in glove box (12 hours). Yield was 3.85 g/79%. The solid was analyzed by XRPD and the XRPD pattern is presented in FIG. 5.

### Example 11: Preparation of Form B of Guadecitabine Sodium

In glovebox in 100 ml round bottom flask equipped with magnetic stirrer, guadecitabine guanidinium (320 mg, 0.519 mmol) was dissolved in 90% aq. MeOH (32 ml) at 50° C. during 10 min. Solution of NaClO₄ hydrate (1.49 g, 10.382 mmol) in MeOH (5 ml) was added in one portion into stirred solution. The formed suspension was stirred at 40° C. for 10 min and slowly cooled to 25° C. during 30 min. Anhydrous ethanol (32 ml) was added dropwise into stirred suspension during 30 min and then the suspension was cooled to 0° C. White solids were filtered on sintered glass filter, washed by anhydrous EtOH (10 ml) then by acetone (10 ml) and dried by flow of nitrogen in glovebox (12 h). Yield was 263 mg (87%). The solid was analyzed by XRPD and the XRPD pattern is presented in FIG. 6.

### Example 12: Preparation of 3′,5′-bis-O-(tert-butyldimethylsilyl)-2′-deoxyguanosine (3a)

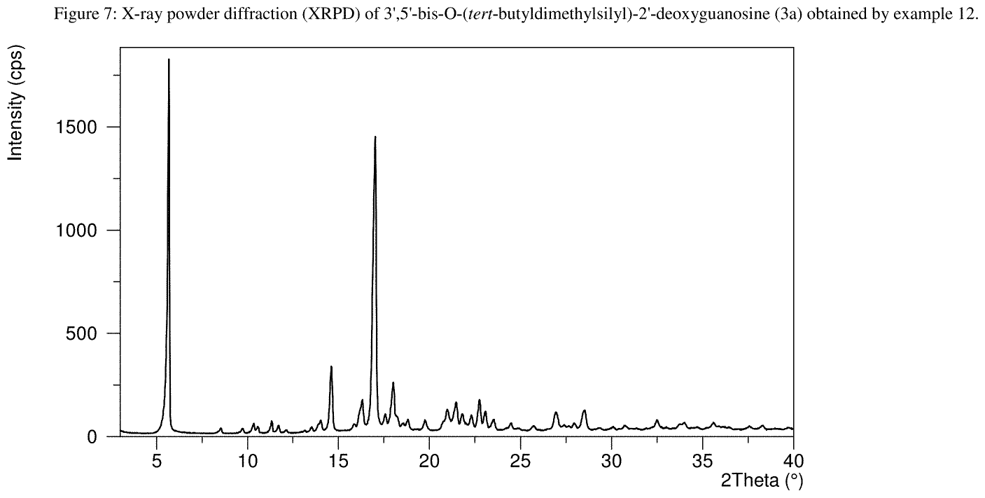

2-deoxyguanosine monohydrate (2790 g, 9.78 mol) was suspended in DMF (16, 7 l, 6 vol) and toluene (14 l, 5 vol) in 301 hastalloy reactor with flat blade turbine impeller. Imidazole (1680 g, 24.65 mol, 2.52 mol eq) was added and the reaction mixture was distilled under reduced pressure (T internal 75° C.) to final volume of 16 l (6 vol). The distillation was repeated with another 14 l of toluene. The suspension was cooled to 20° C. and a suspension of TBSCl (3538 g, 23.47 mol, 2.4 mol eq) in anhydrous DMF (2, 8 l, 1 vol) was added. The suspension was stirred at 45 to 55° C. for 3 h under N₂ atmosphere. The reaction mixture was cooled down to 0° C. and diluted with MeOH/H₂O solvent mixture (1:2 v/v, 8.4 l, 3 vol) at once and stirred for 1 h at 0 to 10° C. The precipitate was filtered off, washed with MeOH (14 l, 5 vol), H₂O (14 l, 5 vol), MeOH (14 l, 5 vol), and dried under vacuum and stream of nitrogen to yield 4681 g (97%) of white crystalline solid. The solid was analyzed by XRPD and the XRPD pattern is presented in FIG. 7.

### Example 13: Preparation of 3′-O-tert-butyldimethylsilyl-2′-deoxyguanosine (4a)

A suspension of 3a, prepared according to example 12, (1600 g, 3.23 mol) in THF (9.6 l, 6 vol) and H₂O (1.2 l, 0.75 vol) was cooled to 0° C. in 301 hastalloy reactor with flat blade turbine impeller. Trifluoroacetic acid (930 ml, 12.1 mol, 3.75 mol eq) diluted in THF (1 l, 1.25 vol) was added during 10 min and the mixture was stirred at 5° C. for 7.5 h. The reaction mixture was neutralized with triethylamine (2360 ml, 5.25 mol eq) while warmed up to 25° C. and stirred at this T for 14 h. The cloudy solution was filtered through Kleenpak Nova Preflow NP6UBP1G to remove insoluble byproducts and rinsed with THF (1.6 l, 1 vol) to 301 hastalloy reactor with flat blade turbine impeller. The solution was diluted with DMF (6.4 l, 4 vol) and H₂O (12 l, 7.5 vol) solvent mixture. The mixture was thickened at atmospheric pressure (T internal 110° C.) to final volume of 20 l (12.5 vol), afterwards was stirred under a cooling ramp from 90° C. to 5° C. during 2 h. The suspension was stirred at 0 to 10° C. for 1 hour. The solid was filtered of washed twice with 90% aqueous n-propanol (8 l, 5 vol), and dried under vacuum at 45° C. for 12 h to yield 740 g of a crude product, which was slurried as follows.

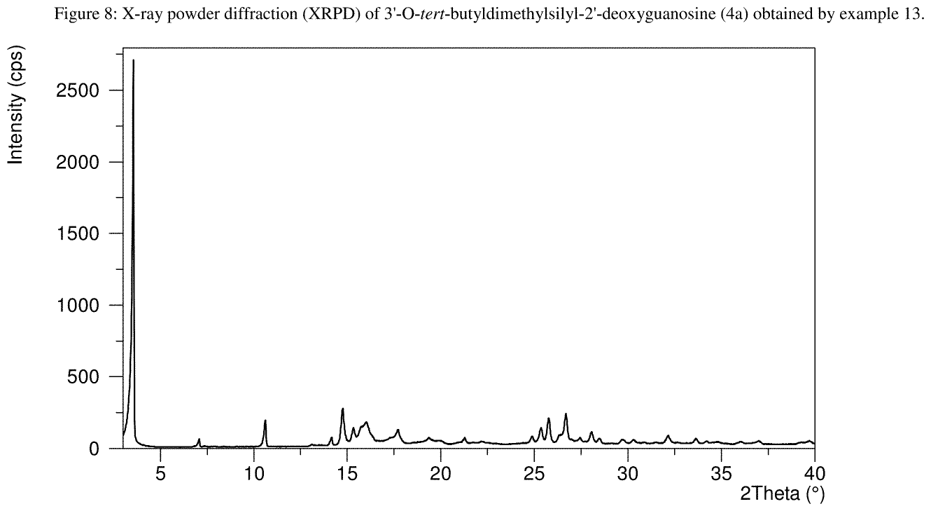

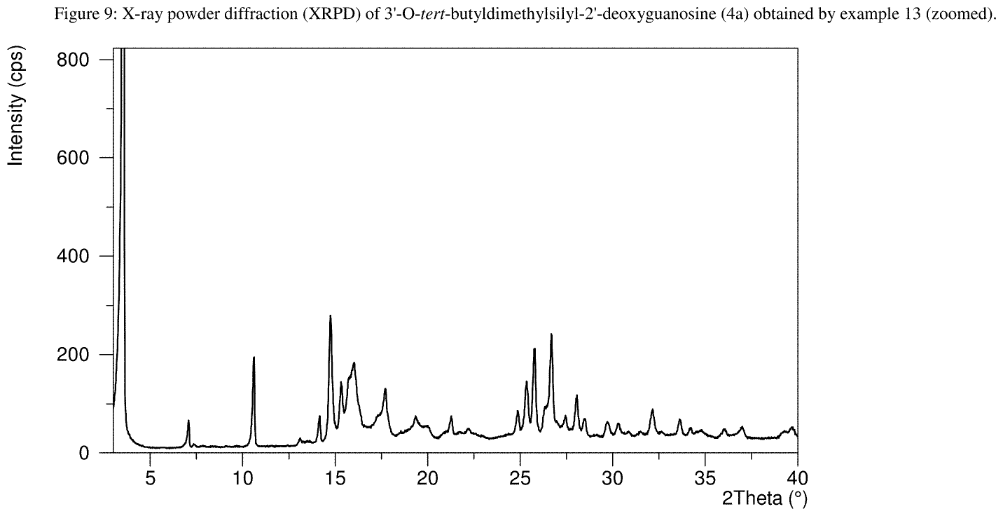

The crude 4a was suspended in DMF/H₂O solvent mixture (2:3 v/v, 9.7 l, 13.1 vol) containing triethylamine (70 ml, 0.484 mol, 0.25 mol eq) in 30 l hastalloy reactor with flat blade turbine impeller, and heated at 90° C. for 3 h. A cooling ramp to 5° C. during 2 h was set, and additionally stirred for 1 h. The solid was filtered off, washed with 90% aq. n-PrOH (3.8 l, 5 vol), and dried under vacuum at 45° C. to yield 704 g (57%, based on the starting 3a) of white crystalline solid. The solid was analyzed by XRPD and the XRPD pattern is presented in FIGS. 8 and 9.

### Example 14: Preparation of 3′-tert-butyldimethylsilyl-2′-deoxyguanosine-5′-O-[(2-cyanoethyl)-N,N-diisopropylphosphoramidite] (5a)

Compound 4a (5 g, 13 mmol) was dissolved in anhydrous DMF (50 ml, 10 vol) in a 100-ml reactor under N₂ atmosphere. Toluene (75 ml, 15 vol) was added and the mixture was distilled under reduced pressure (T internal 70° C.) to the initial volume of 50 ml. The solution was cooled to 23° C. and 2-cyanoethyl-N,N,N′,N′-tetraisopropylphosphorodiamidite (5.83 ml, 18 mmol, 1.4 mol eq) was added followed by a dropwise addition of a solution of N-methylimidazolium trifluoroacetate (2.83 g, 14 mmol, 1.1 mol eq) in anhydrous DMF (10 ml, 2 vol) during 2 h (by using KDS syringe pump). The resulting mixture was stirred at 23° C. for 2 h. The solution was diluted with MTBE (100 ml, 20 vol) and water (25 ml, 5 vol) was added slowly maintaining temperature between 23-25° C. The layers were separated and water (40 ml, 8 vol) was added slowly maintaining temperature between 23-25° C. The layers were separated and the organic layer in the form of a suspension was diluted with n-heptane (150 ml, 30 vol) over 10 min. The suspension was cooled to 5° C. and stirred for 0.5 h. The solid was filtered off, washed with n-heptane (2×25 ml, 5 vol) and dried in the vacuum chamber (24° C., 2 mbar) for 12 h to yield 6.7 g (87%).

### Example 15: Preparation of 5′-tert-butyldimethylsilyl-2′-deoxy-5-azacytidine (7a)

Decitabine (980 g, 4.29 mol) and imidazole (804 g, 11.81 mol, 2.75 mol eq) were suspended in anhydrous DMA (9.8 l, 10 vol) under N₂ atmosphere in glass reactor (30 L, anchor-type stirrer), and heated to 50° C. until complete dissolution. After subsequent cooling down to −15° C., a solution of TBSCl (769 g, 4.96 mol, 1.15 mol eq) in anhydrous DMA (1.96 l, 2 vol) was added dropwise from glass reactor (10 L, anchor-type stirrer). Piping was rinsed with more anh. DMA (0.49 l, 0.5 vol), which was added to the reaction mixture, and continued stirring at −15° C. for 2 h. The cold solution was added slowly to a THF-H₂O solvent mixture (1:5 v/v, 37.73 l, 38.5 vol) to glass reactor (50 L, anchor-type stirrer) under vigorous stirring, keeping T below 30° C. The reactor was rinsed with more DMA (0.98 l, 1 vol) and added to the resulting suspension. A cooling ramp to 0° C. during 1 h was set and stirred for 1 l. The solid was filtered off washed with THF/H₂O solvent mixture (1:5 v/v, 11.76 l, 12 vol), diisopropylether (9.8 l, 10 vol), and dried under vacuum for 12 h to yield 1340 g of white solid. The crude product was purified as follows.

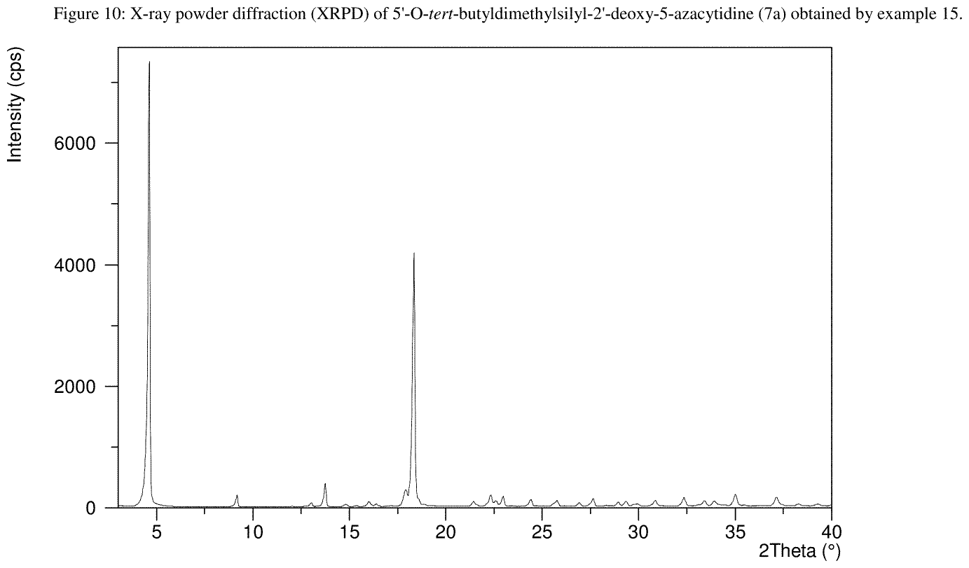

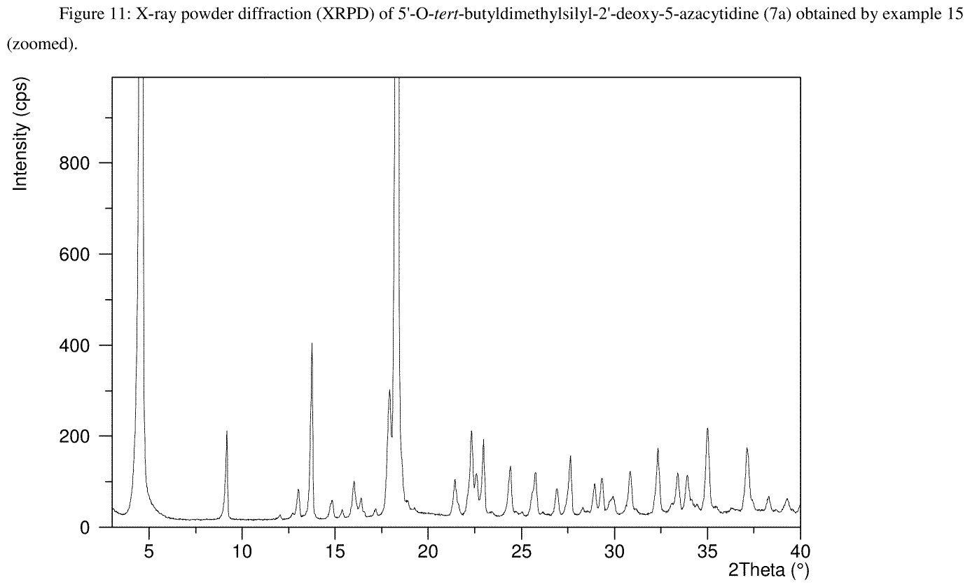

The crude 5a (1340 g) was suspended in AcOEt-MeCN solvent mixture (1:1 v/v, 13.4 l, 10 vol) (which was pretreated with solid K₂CO₃ (670 g, 5 wt % regarding the solvent volume) by stirring for 15 min and removal by filtration). The suspension was stirred at 60° C. for 3 h, then cooled to 0° C. for 0.5 h. The solid was filtered off, washed with AcOEt/MeCN solvent mixture (1:1 v/v, 5.36 l, 4 vol), and dried under vacuum to yield 1095 g (74%, based on the starting decitabine) of white crystalline solid. The solid was analyzed by XRPD and the XRPD pattern is presented in FIGS. 10 and 11.

### Example 16: Preparation of 5′-O-tert-butyldimethylsilyl-2′-deoxy-5-azacytidine-3′-O-[(2-cyanoethyl)-N,N-diisopropylphosphoramidite] (9a)

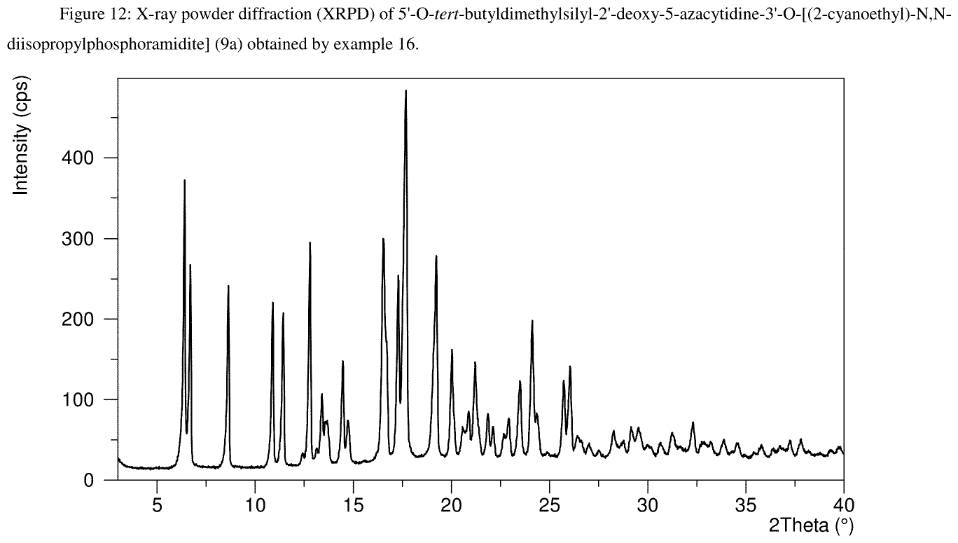

A solution of 7a, prepared according to example 15 (1 kg, 2.92 mol) and 2-cyanoethyl-N,N,N′,N′-tetraisopropylphosphorodiamidite (1.03 kg, 3.36 mol, 1.15 mol eq) in anhydrous DMF (6 l, 6 vol) was cooled in glass reactor (10 L, anchor-type stirrer) to −25° C. under N₂ atmosphere. To this, a solution of N-methylimidazolium trifluoroacetate (0.66 kg, 3.36 mol, 1.15 mol eq) in anhydrous DMF (2 l, 2 vol) was added dropwise during 1 h. The dropping funnel was rinsed with more anh. DMF (0.5 l, 0.5 vol) and added to the reaction mixture, which was stirred for additional 3-5 h under N₂ atmosphere, keeping T below −20° C. This mixture was transferred to glass reactor (50 L, anchor-type stirrer). MTBE (15 l, 15 vol) and H₂O (100 ml, 0.1 vol) were added to the mixture under vigorous stirring and gradual warming up to 25° C. More H₂O (9 l, 9 vol) was at once added to the mixture under stirring to induce crystallization of the product. The resulting suspension was diluted with n-heptane (15 l, 15 vol) over 0.5 h and cooling to 0° C. during 0.5 h. The precipitate was filtered off, washed with H₂O (10 l, 10 vol), n-heptane (25 l, 25 vol), and dried under vacuum and stream of nitrogen for 12 h to yield 1.3 kg (82%) of white crystalline solid. The solid was analyzed by XRPD and the XRPD pattern is presented in FIG. 12.

### Example 17: Preparation of 2-Cyanoethyl-(P-deoxy) (5′-O-(tert-butyldimethylsilyl)-2′-deoxy-5-azacytosylyl)-(3′→5′)-3′-O-(tert-butyldimethylsilyl)-2′-deoxyguanosine (10a) and 2-Cyanoethyl (5′-O-(tert-butyldimethylsilyl)-2′-deoxy-5-azacytosylyl)-(3′→5′)-3′-O-(tert-butyldimethylsilyl)-2′-deoxyguanosine (11a) via 4a and 9a

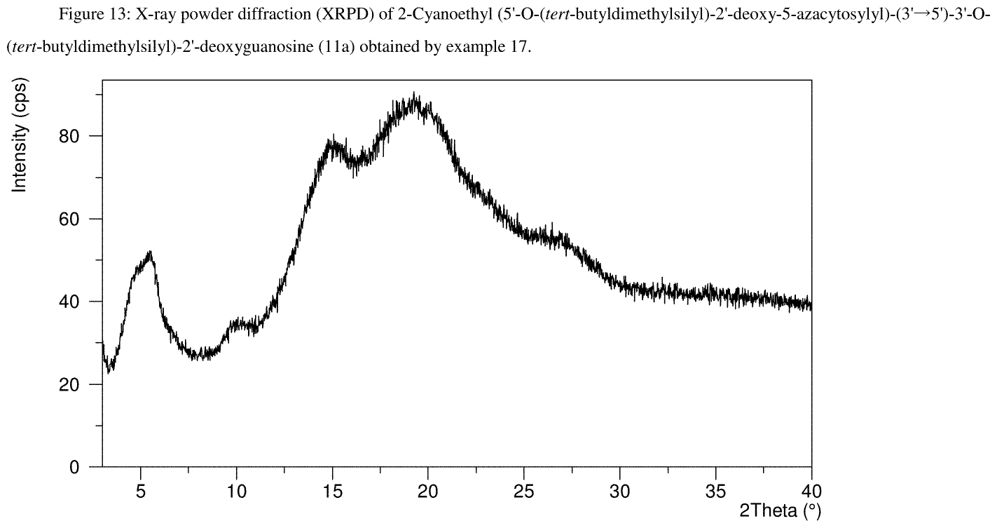

Compounds 4a (225 g, 0.58 mol) and 9a (353 g, 0.58 mol, 1 mol eq) were dissolved in DMF (5.6 l, 25 vol regarding 4a) in 13 l glass reactor with anchor impeller. Toluene (5.6 l, 25 vol regarding 4a) was added and the solution was distilled under reduced pressure (T internal should not exceed 40° C.) to the initial volume of the DMF solution. This process was repeated once more, and finally kept under N₂ atmosphere. The solution was cooled to 0° C. at which a solution of N-methylimidazolium trifluoroacetate (143 g, 0.729 mol, 1.25 mol eq) in anhydrous DMF (450 ml, 2 vol regarding 4a) was added slowly during 45 min. The reaction mixture was stirred at 0° C. under N₂ atmosphere for 17 h. The resulting 10a was directly oxidized by addition of tert-butylhydrogenperoxide, 5.5 M in nonane (186 mL, 1.02 mol, 1.75 mol eq). The mixture was warmed up to 20° C. and stirred for 2 h. After completing the reaction, MeCN/H₂O solvent mixture (1:5 v/v, 6.0 l, 27 vol) was added during 45 minutes, keeping T below 25° C. The resulting suspension was cooled to 15° C. and stirred at this T for 18 h. The precipitate was filtered off, washed with MeCN/H₂O solvent mixture (1:5 v/v, 1.25 l, 6 vol), MTBE (2.7 l, 12 vol), and dried under vacuum at 30° C. for 12 h affording 503 g (95%) of crude 11a as a white amorphous solid (HPLC purity 94 wt %). The solid was analyzed by XRPD and the XRPD pattern is presented in FIG. 13.

### Example 18: Preparation of Guadecitabine Triethylammonium Salt Via 4a and 9a

To a solution of 11a, prepared according to example 17, (475 g, 0.57 mol) in anhydrous DMA (4.75 l, 10 vol), containing DMSO (238 ml, 0.5 vol) in 15 l glass reactor with anchor impeller, Et₃N (316 mL, 2.27 mol, 4 mol eq) and subsequently 3HF.Et₃N complex (185 ml, 1.14 mol, 2 mol eq) were added. The solution was gently heated at 50° C. under nitrogen atmosphere for 27.5 h. After completing the deprotection, this solution was added during 0.5 h to a stirring MeCN (14.25 l, 30 vol) in 301 hastalloy reactor with flat blade turbine impeller precooled to 15° C. The reactor was rinsed with more DMA (475 ml, 1 vol) and added to the resulting suspension. The precipitate was filtered off, washed with MeCN (4.75 l, 10 vol), MTBE (2.38 l, 5 vol), and dried under vacuum at 40° C. for 12 h to yield 366 g (92%) of crude guadecitabine triethylammonium salt as a white solid, HPLC purity 98.06 wt %.

### Example 19: Preparation of Guadecitabine Guanidinium Salt

Guadecitabine triethylammonium salt prepared according to the procedure of example 18 (60 g, 91 mmol) was suspended in the mixture of MeOH (1.80 l, 30 vol) and water (420 ml, 7 vol) in 101 glass reactor equipped with anchor type stirrer in glove box under nitrogen atmosphere. The mixture was tempered to 45° C. to dissolve completely. Guanidine carbonate (8.62 g, 53 mmol) dissolved in water (60 ml, 1 vol) was added at once into stirred solution. Solution was cooled to 25° C. during 15 min and stirred at 25° C. for next 1 h. Crystallization was spontaneously induced. Then AcN (1920 ml, 32 vol) was added during 20 min into stirred suspension. The resulting suspension was stirred and cooled to 5° C. during 2 h and stirred further at 5° C. for next 3 h. The solids were filtered off, washed with 90% aq. EtOH (480 ml, 8 vol) and dried under vacuum and stream of nitrogen in a vacuum oven for 12 h at 30° C. The isolated yield was 40.2 g (75%), HPLC purity 99.76 wt %.

The solid was analyzed by XRPD and identified as form G1 and the XRPD pattern is presented in 14.

### Example 20: Preparation of Guadecitabine Sodium

Guadecitabine guanidinium (39 g) prepared according to example 19 was milled at Frewitt (sieve 1 μm, milling speed 10,000 RPM, dosing speed 10 RPM, in glovebox under nitrogen atm.) to obtain 25.6 g. Milled guadecitabine guanidinium (25.6 g, 41 mmol) was suspended in the mixture of MeOH (1152 ml, 45 vol) and water (128 ml, 5 vol) in 101 glass reactor equipped with anchor type stirrer in glovebox under nitrogen atm. Formed suspension was heated to 40° C. Then sodium acetate trihydrate (28.18 g, 207 mmol) dissolved in MeOH (128 ml, 5 vol) was added and suspension was stirred for next 3 h at 40° C. The suspension was cooled down to 0° C. during 2 h and filtered. Solids were washed by MeOH (1 l, 25 vol). Product was dried in vacuum oven at 30° C. for 12 h to obtain 24.0 g (75%), HPLC purity 99.52 wt %. The solid was analyzed by XRPD and the XRPD pattern is presented in FIG. 15.

### Example 21: Preparation of 2-Cyanoethyl-(P-deoxy) (5′-O-(tert-butyldimethylsilyl)-2′-deoxy-5-azacytosylyl)-(3′→5′)-3′-O-(tert-butyldimethylsilyl)-2′-deoxyguanosine (10a) and 2-Cyanoethyl (5′-O-(tert-butyldimethylsilyl)-2′-deoxy-5-azacytosylyl)-(3′→5′)-3′-O-(tert-butyldimethylsilyl)-2′-deoxyguanosine (11a) via 7a and 5a

Compound 5a (14 g, 20.8 mmol, 1.2 mol eq) was dissolved in anhydrous DMF (77 ml, 12 vol) in a 250-ml reactor under N₂ atmosphere (glovebox). Toluene (90 ml, 15 vol) was added and the mixture was distilled under reduced pressure (65-80 mbar, T internal should not exceed 32° C.) to the initial volume of 77 ml. The solution was cooled to 23° C., solid 7a (6.4 g, 17.3 mmol) was added followed by rinse with anhydrous DMF (13 ml, 2 vol). A solution of pyridinium trifluoroacetate (4 g, 20.8 mmol, 1.2 mol eq) in anhydrous DMF (20 ml, 3 vol) was added dropwise during 15 min and the reaction mixture was stirred for 17 h. The resulting 10a was directly oxidized by addition of tert-butylhydrogenperoxide, 5.5M in nonane (4.7 ml, 26 mmol, 1.5 mol eq) and the reaction mixture was stirred at 23° C. for 1 h. The solution was cooled to 15° C. and water (150 ml, 23 vol) was added dropwise maintaining temperature below 25° C. The suspension was cooled to 10° C. and stirred at 10° C. for 0.5 h. The solid was filtered off washed with water (2×120 ml, 19 vol), MTBE (120 ml, 19 vol), pre-dried by stream of N₂ (glovebox) and dried in vacuum chamber (40° C.) for 17 h to yield 16 g (87%) of crude 11a as a white amorphous solid (HPLC purity 79 wt %).

### Example 22: Preparation of Form G2 of Guadecitabine Guanidinium Salt

Guadecitabine triethylammonium salt prepared according to the procedure of example 18 (1.0 g, 1.5 mmol) was suspended in the mixture of MeOH (50 ml, 30 vol) and water (7 ml, 7 vol) in 100 ml round bottom flask equipped with magnetic stirrer. The mixture was tempered to 45° C. to dissolve completely. Then solution was clarified by filtration and guanidine carbonate (0.14 g, 0.78 mmol) dissolved in water (1 ml, 1 vol) was added at once into stirred solution at 35° C. Solution was let at ambient temperature (approx. 22° C.) for next 3 h. The solids were filtered off, washed with MeOH (5 ml, 5 vol) and dried under vacuum and stream of nitrogen in a vacuum oven for 4 h at 30° C. The isolated yield was 0.5 g (53%), HPLC purity N/A.

The solid was analyzed by XRPD and the XRPD pattern is presented in FIG. 20.

### Example 23: Comparison of Guadecitabine Sodium Form A Stability in Water at 25° C. and Methanol Water (9/1 v/v) at 40° C.

In water stability of guadecitabine sodium form A was measured as follows.

Instrument method: autosampler temp. was set to 25° C. 10 mg of guadecitabine sodium form A was weighted in 2-mL vial in glovebox and 1 mL of water was added to have concentration exactly 10 mg/mL. Sample was injected freshly after preparation (0 min), 20 min after preparation (20 min) and afterwards every 40 min (duration of single analysis run).
The results are shown in Table 2.

TABLE 2

| | Injection | HPLC contents (%) | | | | |
|---|---|---|---|---|---|---|
| | time point | guadecitabine | Degradant | Degradant | Degradant | Degradant |
| Sample | [min] | sodium | 2a | 2b | 2c | 2d |
| Solution in | — | 99.82 | <0.05 | <0.05 | 0.14 | 0.05 |
| DMSO | | | | | | |
| Solution in | 0 | 99.80 | <0.05 | <0.05 | 0.13 | 0.07 |
| water | 20 | 99.63 | <0.05 | <0.05 | 0.14 | 0.23 |
| | 40 | 99.39 | <0.05 | 0.06 | 0.16 | 0.39 |
| | 80 | 99.02 | <0.05 | 0.11 | 0.21 | 0.67 |
| | 120 | 98.58 | <0.05 | 0.13 | 0.31 | 0.99 |
| | 160 | 98.16 | <0.05 | 0.18 | 0.45 | 1.20 |
| | 200 | 97.56 | <0.05 | 0.25 | 0.58 | 1.61 |
| | 240 | 97.11 | 0.05 | 0.25 | 0.75 | 1.85 |
| | 280 | 96.29 | 0.07 | 0.29 | 0.89 | 2.47 |
| Suspension in | 240 | 99.76 | <0.05 | <0.05 | 0.14 | 0.10 |
| methanol/water | | | | | | |
| (9/1) | | | | | | |

1. Crystalline Guadecitabine sodium.

2. The crystalline Guadecitabine sodium according to claim 1, selected from:
(A) crystalline Guadecitabine sodium designated form A, characterized by data selected from one or more of the following:
(i) an X-ray powder diffraction pattern having peaks at 10.0, 11.3, 12.2, 20.1 and 21.0 degrees two theta±0.2 degrees two theta;
(ii) an X-ray powder diffraction pattern having peaks at 10.0, 11.3, 12.2, 20.1 and 21.0 degrees two theta±0.2 degrees two theta and also having any one, two, three, four or five additional peaks selected from 4.9, 14.8, 16.5, 18.7 and 29.7 degrees two theta±0.2 degrees two theta;
(iii) an X-ray powder diffraction pattern substantially as depicted in FIG. 5 or in FIG. 15;
(iv) an FT-IR spectrum having absorptions at 1674, 1080, 1018, 949 and 782 cm⁻¹ 4 cm¹;
(v) an FT-IR spectrum substantially as depicted in FIG. 16 or in FIG. 17;
(vi) a solid state ¹³C NMR spectrum having peaks at 166.9, 159.1, 156.0, 154.7 and 151.6 ppm±0.2 ppm;
(vii) a solid state ¹³C NMR spectrum having the following chemical shift absolute differences between said characteristic peaks at 166.9, 159.1, 156.0, 154.7 and 151.6 ppm±0.2 ppm and a reference peak at 114.7 ppm±1 ppm of 52.2, 44.4, 41.3, 40.0 and 36.9 ppm±0.1 ppm; and
(viii) a solid state ¹³C NMR spectrum substantially as depicted in FIG. 18 or in FIG. 19;
and
(B) crystalline Guadecitabine sodium designated form B, characterized by data selected from one or more of the following:
(i) an X-ray powder diffraction pattern having peaks at 10.8, 12.2, 20.3, 23.3 and 26.7 degrees two theta±0.2 degrees two theta; and
(ii) an X-ray powder diffraction pattern having peaks at 10.8, 12.2, 20.3, 23.3 and 26.7 degrees two theta±0.2 degrees two theta and also having any one, two, three or four additional peaks selected from 14.1, 15.4, 16.8 and 30.3 degrees two theta±0.2 degrees two theta.

3. The crystalline Guadecitabine sodium according to claim 2, wherein said crystalline Guadecitabine sodium is isolated in solid form, or wherein said crystalline Guadecitabine sodium is substantially free of any other solid state forms.

4. Guadecitabine sodium which contains: about 0.15 wt % or less, preferably about 0.10 wt % or less, more preferably 0.05 wt % or less as measured by HPLC of each one of degradant 2a and degradant 2b and/or contains about 0.25 wt % or less, preferably about 0.20 wt % or less, more preferably 0.15 wt % or less as measured by HPLC of each one of degradant 2c and degradant 2d:

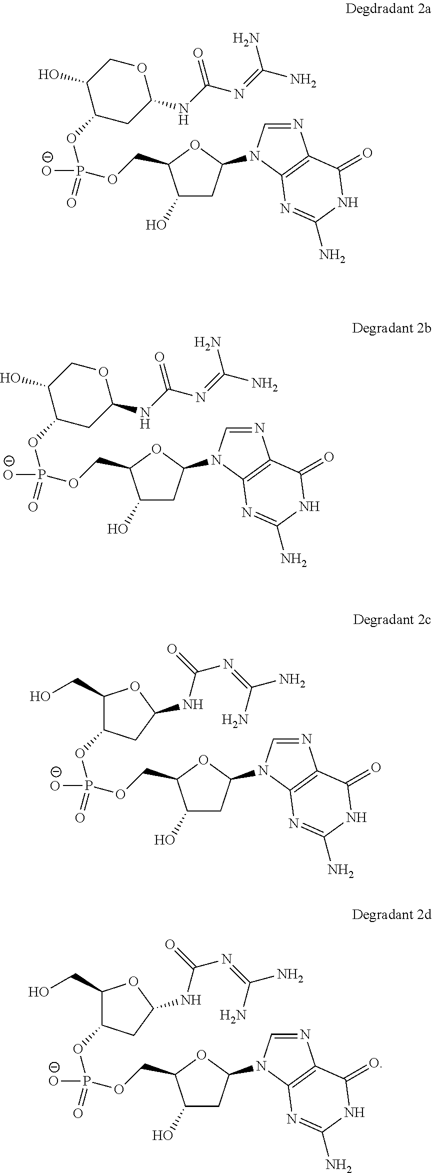 [SMILES: NC(N)=NC(=O)N[C@@H]1C[C@H](OP(=O)([O-])OC[C@H]2O[C@@H](n3cnc4c(=O)[nH]c(N)nc43)C[C@@H]2O)[C@H](O)CO1 ; NC(N)=NC(=O)N[C@H]1C[C@H](OP(=O)([O-])OC[C@H]2O[C@@H](n3cnc4c(=O)[nH]c(N)nc43)C[C@@H]2O)[C@H](O)CO1 ; NC(N)=NC(=O)N[C@H]1C[C@H](OP(=O)([O-])OC[C@H]2O[C@@H](n3cnc4c(=O)[nH]c(N)nc43)C[C@@H]2O)[C@@H](CO)O1 ; NC(N)=NC(=O)N[C@@H]1C[C@H](OP(=O)([O-])OC[C@H]2O[C@@H](n3cnc4c(=O)[nH]c(N)nc43)C[C@@H]2O)[C@@H](CO)O1]

5. Guadecitabine sodium which has a total impurity content of: about 0.9 wt % or less, about 0.5 wt % or less, about 0.3 wt % or less, preferably about 0.2 wt % or less, most preferably about 0.1 wt % or less, as measured by HPLC.

6. The Guadecitabine sodium according to any one of claims 4-5, wherein said Guadecitabine sodium is amorphous.

7. The Guadecitabine sodium according to any one of claims 4-5, wherein said Guadecitabine sodium is crystalline, preferably wherein the crystalline form is form A or form B according to any one of claims 2-3.

8. Guadecitabine Guanidinium.

9. The Guadecitabine guanidinium according to claim 8, wherein said guadecitabine guanidinium is in a solid state form selected from:
(A) a form of Guadecitabine guanidinium designated form G1, characterized by data selected from one or more of the following:
(i) an X-ray powder diffraction pattern having peaks at 11.1, 23.3 and 25.8 degrees two theta±0.2 degrees two theta;
(ii) an X-ray powder diffraction pattern having peaks at 11.1, 13.5, 16.9, 23.3 and 25.8 degrees two theta±0.2 degrees two theta; and
(iii) an X-ray powder diffraction pattern having peaks at 11.1, 13.5, 16.9, 23.3 and 25.8 degrees two theta±0.2 degrees two theta and also having any one, two, three or four additional peaks selected from 4.5, 20.8, 21.6 and 28.6 degrees two theta±0.2 degrees two theta;
and
(B) a crystalline form of Guadecitabine guanidinium designated form G2, characterized by data selected from one or more of the following:
(i) an X-ray powder diffraction pattern having peaks at 4.5, 17.3, 21.6, 24.3 and 27.5 degrees two theta±0.2 degrees two theta; and
(ii) an X-ray powder diffraction pattern having peaks at 4.5, 17.3, 21.6, 24.3 and 27.5 degrees two theta±0.2 degrees two theta and also having any one, two, three, four or five additional peaks selected from 8.9, 9.7, 13.6, 17.9 and 21.3 degrees two theta±0.2 degrees two theta.

10. The Guadecitabine guanidinium according to claim 9, wherein the crystalline form is isolated in solid form, or wherein the crystalline form is substantially free of any other solid state forms.

11. The Guadecitabine guanidinium according to claim 8 which contains: about 0.15 wt % or less, preferably about 0.10 wt % or less, more preferably about 0.05 wt % or less as measured by HPLC of each one of degradant 2a and degradant 2b and/or contains about 0.25 wt % or less, preferably about 0.20 wt % or less, more preferably 0.15 wt % or less as measured by HPLC of each one of degradant 2c and degradant 2d:

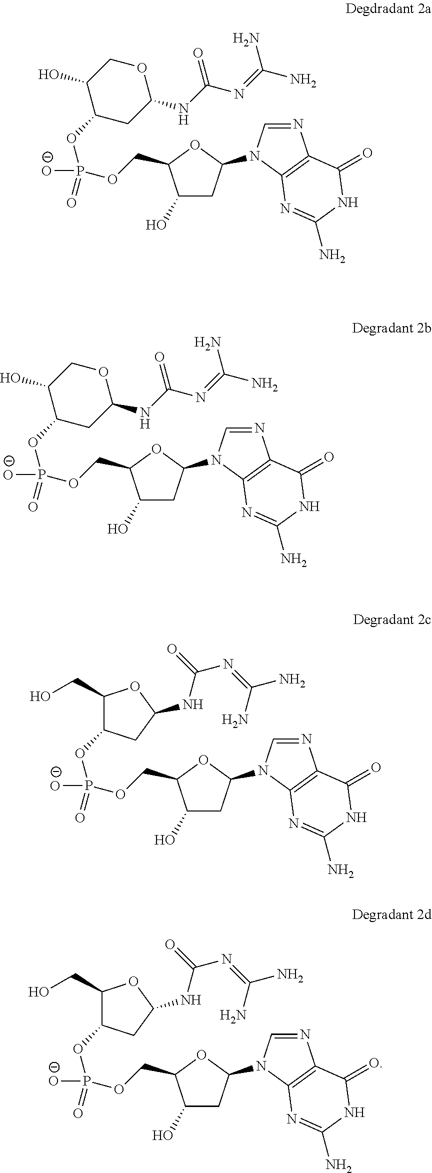 [SMILES: NC(N)=NC(=O)N[C@@H]1C[C@H](OP(=O)([O-])OC[C@H]2O[C@@H](n3cnc4c(=O)[nH]c(N)nc43)C[C@@H]2O)[C@H](O)CO1 ; NC(N)=NC(=O)N[C@H]1C[C@H](OP(=O)([O-])OC[C@H]2O[C@@H](n3cnc4c(=O)[nH]c(N)nc43)C[C@@H]2O)[C@H](O)CO1 ; NC(N)=NC(=O)N[C@H]1C[C@H](OP(=O)([O-])OC[C@H]2O[C@@H](n3cnc4c(=O)[nH]c(N)nc43)C[C@@H]2O)[C@@H](CO)O1 ; NC(N)=NC(=O)N[C@@H]1C[C@H](OP(=O)([O-])OC[C@H]2O[C@@H](n3cnc4c(=O)[nH]c(N)nc43)C[C@@H]2O)[C@@H](CO)O1]

12. The Guadecitabine guanidinium according to claim 8 or 11 which has a total impurity content of: about 0.9 wt % or less, about 0.5 wt % or less, about 0.3 wt % or less, preferably about 0.2 wt % or less, most preferably about 0.1 wt % or less, as measured by HPLC.

13. The Guadecitabine guanidinium according to any one of claims 11-12, wherein the guadecitabine guanidinium is amorphous.

14. The Guadecitabine guanidinium according to any one of claims 11-12, wherein the guadecitabine guanidinium is crystalline or semi-crystalline, preferably wherein the guadecitabine guanidinium is guadecitabine guanidinium of form G1 or G2 according to any one of claims 9-10.

15. Use of Guadecitabine guanidinium according to any one of claims 8-14, in the preparation of Guadecitabine or other salts thereof, preferably guadecitabine sodium.

16. Use of Guadecitabine guanidinium according to any one of claims 8-14, in the preparation of pharmaceutical composition comprising Guadecitabine or a salt thereof, preferably guadecitabine sodium.

17. The use according to any one of claims 15-16, wherein the guadecitabine sodium is guadecitabine sodium according to any one of claims 1-7.

18. Guadecitabine guanidinium according to any one of claims 8-14 for use in the preparation of Guadecitabine or other salts thereof, preferably guadecitabine sodium.

19. Guadecitabine guanidinium according to any one of claims 8-14 for use in the preparation of a pharmaceutical composition comprising Guadecitabine or a salt thereof, preferably guadecitabine sodium.

20. Guadecitabine guanidinium for the use according to any one of claims 18-19, wherein guadecitabine sodium is according to any one of claims 1-7.

21. A process for preparation of Guadecitabine or salt thereof, preferably guadecitabine sodium, which process comprises:
a) providing guadecitabine guanidinium salt;
b) optionally heating the reaction mixture;
c) converting guadecitabine guanidinium to guadecitabine sodium;
d) optionally cooling the reaction mixture;
e) optionally separating the solid guadecitabine sodium formed; and
f) optionally washing and/or drying of said guadecitabine sodium separated in step e).

22. The process according to claim 21, wherein step a) comprises providing guadecitabine guanidinium in a mixture of a polar solvent and water, preferably alcohol and water, more preferably methanol and water.

23. The process according to claim 22, wherein the amount of water is NMT about 50% v/v, NMT about 20% v/v, NMT about 10% v/v, or NMT about 5% v/v, preferably wherein the amount of water is NMT about 30% v/v, more preferably NMT about 20% v/v, even more preferably NMT about 10% v/v, and most preferably from about 5% v/v to about 10% v/v.

24. The process according to any one of claims 21-23, wherein step c) comprises adding a sodium salt, preferably sodium acetate.

25. The process according to claim 24, wherein the sodium acetate salt is dissolved in methanol prior to addition to the reaction mixture.

26. The process according to any one of claims 21-25, wherein:
in step a) Guadecitabine guanidinium is provided in a mixture of methanol and water wherein the amount of water is NMT about 50% v/v, NMT about 20% v/v, NMT about 10% v/v, or NMT about 5% v/v, preferably wherein the amount of water is NMT about 30% v/v, more preferably NMT about 20% v/v, even more preferably NMT about 10% v/v, and most preferably from about 5% v/v to about 10% v/v;
in step b) the reaction mixture is optionally heated to a temperature of about 30° C. to about 50° C.;
in step c) sodium acetate is added, preferably in the form of a solution in methanol; and
in step e) Guadecitabine sodium is separated.

27. A process for preparation of Guadecitabine or salt thereof, preferably guadecitabine sodium, more preferably crystalline Guadecitabine sodium, which process comprises:
a) providing Guadecitabine triethylammonium salt optionally in the form of solution;
b) adding guanidine or guanidinium salt, optionally in the form of solution, to form a suspension;
c) optionally separating Guadecitabine guanidinium;
d) providing Guadecitabine guanidinium, optionally in the form of solution, and adding a sodium salt, optionally in the form of solution;
e) adding antisolvent; and
f) optionally isolating Guadecitabine sodium.

28. The process according to claim 27, wherein step a) comprises providing Guadecitabine triethylammonium in a mixture of a polar solvent and water, preferably alcohol and water, more preferably methanol and water.

29. The process according to claim 28 wherein the amount of water is NMT about 50% v/v, NMT about 20% v/v, NMT about 10% v/v, or NMT about 5% v/v, preferably wherein the amount of water is NMT about 40% v/v, more preferably NMT about 30% v/v, and most preferably from about 10% v/v to about 30% v/v.

30. The process according to any one of claims 27-29 wherein in step b) guanidine carbonate is used, preferably in the form of a solution in water.

31. The process according to any one of claims 27-30 wherein step d) comprises providing Guadecitabine guanidinium in a mixture of a polar solvent and water, preferably alcohol and water, more preferably methanol and water.

32. The process according to claim 31 wherein the amount of water is NMT about 50% v/v, NMT about 20% v/v, NMT about 10% v/v, or NMT about 5% v/v, preferably wherein the amount of water is NMT about 30% v/v, more preferably NMT about 20% v/v, even more preferably NMT about 10% v/v, and most preferably from about 5% v/v to about 10% v/v.

33. The process according to any one of claims 27-32 wherein in step d) the sodium salt is sodium acetate.

34. The process according to claim 33 wherein the sodium acetate salt is dissolved in methanol prior to addition to the reaction mixture.

35. The process according to claims 27-34 wherein the antisolvent is methanol.

36. A process for preparation of Guadecitabine sodium, more preferably crystalline Guadecitabine sodium, which process comprises:
a) providing Guadecitabine triethylammonium salt optionally in the form of solution;
b) optionally heating the reaction mixture to a temperature of from about 30° C. to about 50° C.;
c) adding guanidine or guanidine salt optionally in the form of a solution;
d) optionally cooling the reaction mixture to a temperature of from about 0° C. to about 25° C.;
e) optionally adding antisolvent;
f) optionally isolating Guadecitabine guanidinium;
g) providing Guadecitabine guanidinium in a mixture of a polar solvent and water;
h) optionally heating the reaction mixture to a temperature of from about 30° C. to about 50° C.;
i) adding a sodium salt, preferably sodium acetate, optionally in the form of a solution in the form of a solution;
j) optionally cooling the reaction mixture to a temperature of from about 0° C. to about 25° C.;
k) optionally adding antisolvent;
l) separating guadecitabine sodium; and
m) optionally washing or drying.

37. The process according to claim 36 wherein step a) comprises providing guadecitabine triethylammonium in a mixture of a polar solvent and water, preferably alcohol and water, more preferably methanol and water.

38. The process according to claim 37 wherein the amount of water is NMT about 50% v/v, NMT about 20% v/v, NMT about 10% v/v, or NMT about 5% v/v, preferably wherein the amount of water is NMT about 40% v/v, more preferably NMT about 30% v/v, and most preferably from about 10% v/v to about 30% v/v.

39. The process according to any one of claims 36-38 wherein step c) comprises adding a guanidine salt, preferably guanidine carbonate.

40. The process according to claim 39 wherein the guanidine carbonate is dissolved in water prior to addition to the reaction mixture.

41. The process according to claim 36-40 wherein in step g) the mixture of a polar solvent and water is preferably a mixture of alcohol and water, more preferably a mixture of methanol and water.

42. The process according to claim 41 wherein the amount of water is NMT about 50% v/v, NMT about 20% v/v, NMT about 10% v/v, or NMT about 5% v/v, preferably wherein the amount of water is NMT about 30% v/v, more preferably NMT about 20% v/v, even more preferably NMT about 10% v/v, and most preferably from about 5% v/v to about 10% v/v.

43. The process according to any one of claims 36-42 wherein step i) comprises adding sodium acetate.

44. The process according to claims 36-43 wherein in step i) the sodium acetate salt is dissolved in methanol prior to addition to the reaction mixture.

45. A process for preparation of Guadecitabine or salt thereof wherein the process comprises:
a) oxidation of compound 10 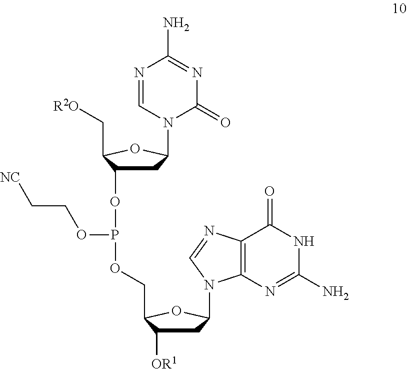
to afford compound 11 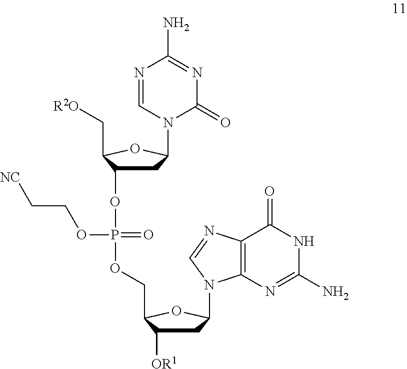
wherein R¹ and R² may be the same or different hydroxy protecting group, preferably selected from —SiR⁷R⁸R⁹, wherein R⁷, R⁸ and R⁹ are independently selected from the group consisting of: C1-C15 straight or branched alkyl, C1-C10 cycloalkyl, optionally substituted C6-C10 aryl, and optionally substituted C7-C12 arylalkyl, preferably t-butyldimethylsilyl (TBDMS), triethylsilyl (TES), t-butyldiphenylsilyl (TBDPS), triisopropylsilyl (TIPS), and trimethylsilyl (TMS); or tetrahydropyranyl (THP), methoxymethyl (MOM), benzyloxymethyl; ethoxyethyl, benzyl or tert-butyl; phenoxyacetate (pac), tert-butylphenoxyacetate (tac), benzoate or substituted benzoate groups; carbonates, preferably benzyloxycarbonate (CBZ), tert-butyloxycarbonate (BOC), fluorenylmethoxycarbonate (FMOC); and acetals, preferably methoxymethyl (MOM), 2-methoxyethoxymethyl (MEM), benzyloxymethyl, or tetrahydropyranyl (THP); and
b) conversion of compound 11 to Guadecitabine or salts thereof.

46. The process according to claim 45 wherein step a) is carried out in the presence of an oxidation reagent, preferably iodine and hydroperoxide, most preferably iodine and t-butyl hydroperoxide (t-BuOOH).

47. The process according to any one of claims 45-46 wherein the process is performed under anhydrous conditions.

48. The process according to any one of claims 45-47 wherein step b) comprises the following steps:
iv) deprotecting compound 11;
v) forming of a basic salt A of Guadecitabine and optionally isolating the salt;
vi) optionally converting the basic salt A formed in step v) to a basic salt B; and
vii) optionally converting the basic salt B formed in step vi) to Guadecitabine or another basic salt thereof

49. The process of claim 48 wherein in step v) guadecitabine triethylammonium is formed as the basic salt A.

50. The process of any one of claims 48-49 wherein in step vi) guadecitabine triethylammonium is converted to guadecitabine guanidinium as the basic salt B.

51. The process of any one of claims 48-50 wherein in step vii) guadecitabine guanidinium salt is converted to Guadecitabine or salt thereof, preferably to Guadecitabine sodium.

52. The process of claim 51 wherein Guadecitabine guanidinium formed in step vi) is crystalline, semi crystalline or amorphous, preferably it is Guadecitabine guanidinium designated as form G1, or form G2.

53. The process of any one of claims 48-52 wherein in step vii) guadecitabine sodium is formed.

54. The process claim 53 wherein crystalline form A and/or B of guadecitabine is formed.

55. The process according to any one of claims 48-54 wherein step iv) is performed in the presence of a base and a fluoride reagent.

56. The process according to claim 55 wherein the fluoride reagent is a salt of hydrogenfluoride and an organic base, preferably TBAF and Et₃N*3HF, most preferably Et₃N*3HF.

57. The process of any one of claims 48-56 wherein step iv) is performed in a mixture of DMA and DMSO, preferably the ratio of DMA:DMSO is about 4:1 v/v, preferably about 6:1 v/v to about 10:1 v/v, more preferably about 20:1 v/v to about 30:1 v/v.

58. The process according to any one of claims 45-57 wherein R¹ and R² are both TBDMS groups.

59. A process for preparation of Guadecitabine sodium which process comprises:
a) oxidizing of compound 10, preferably compound 10a to afford compound 11, preferably 11a 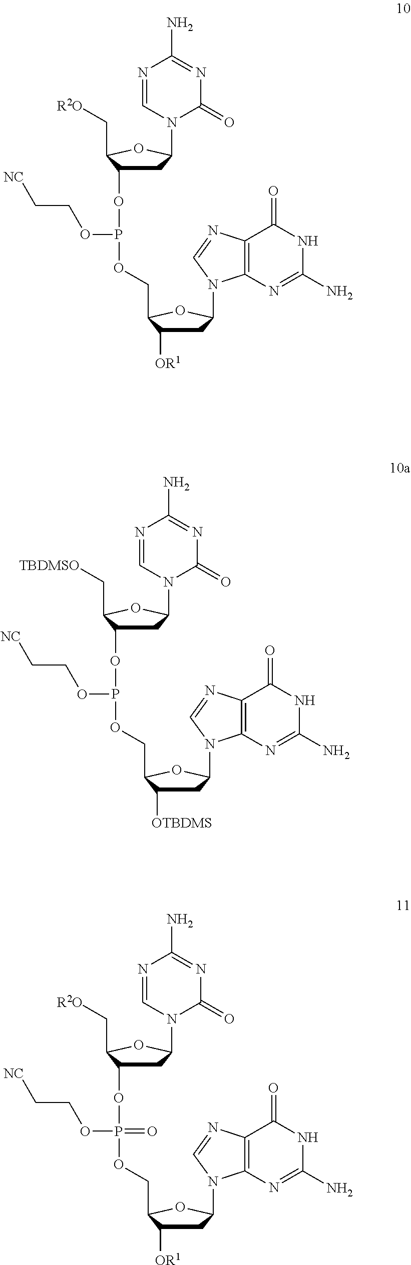 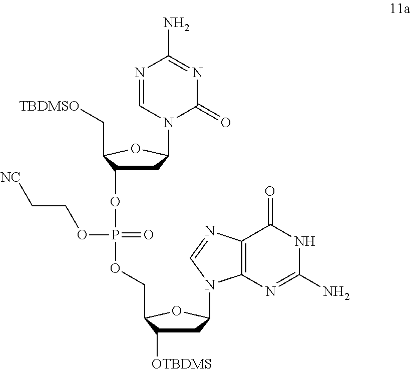
b) deprotecting compound 11 or preferably 11a in the presence of a suitable base, a fluoride reagent and a solvent wherein the solvent comprises DMA and DMSO, the base is triethylamine to afford a triethylammonium salt of Guadecitabine;
c) providing the triethylammonium salt of Guadecitabine in a mixture of an alcohol and water, preferably methanol and water, more preferably wherein the amount of water is NMT about 50% v/v, NMT about 20% v/v, NMT about 10% v/v, or NMT about 5% v/v, preferably wherein the amount of water is NMT about 40% v/v, more preferably NMT about 30% v/v, and most preferably from about 10% v/v to about 30% v/v;
d) adding a guanidine salt, preferably guanidine carbonate, more preferably a solution of guanidine carbonate in water and optionally adding an antisolvent to afford Guadecitabine guanidinium, preferably in crystalline form;
e) providing the Guadecitabine guanidinium in a mixture of an alcohol and water, preferably methanol and water, more preferably wherein the amount of water is NMT about 50% v/v, NMT about 20% v/v, NMT about 10% v/v, or NMT about 5% v/v, preferably wherein the amount of water in the solvent system is NMT about 30% v/v, more preferably NMT about 20% v/v, even more preferably NMT about 10% v/v, and most preferably from about 5% v/v to about 10% v/v; and
f) adding a sodium salt, preferably sodium acetate, more preferably a solution of sodium acetate in methanol and optionally adding an antisolvent to afford Guadecitabine sodium, preferably in crystalline form.

60. A process for preparation of compound 10, wherein the process comprises:
A) coupling compound A 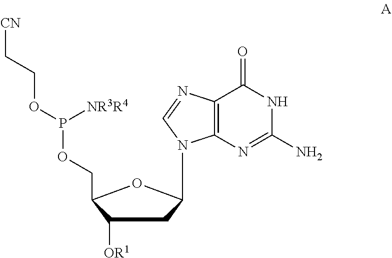
and compound 7 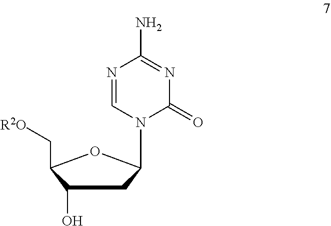
or
B) coupling compound 4 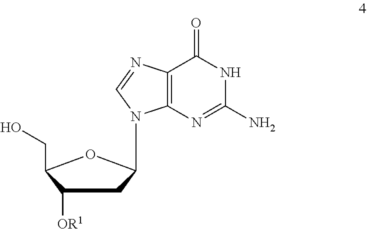
and compound B 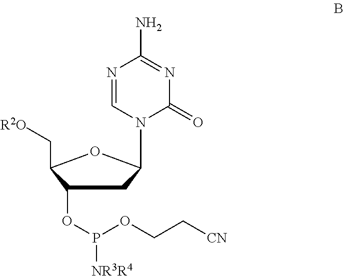
wherein R¹ and R² may be the same or different hydroxy protecting group, preferably selected from —SiR⁷R⁸R⁹, wherein R⁷, R⁸ and R⁹ are independently selected from the group consisting of: C1-C15 straight or branched alkyl, C1-C10 cycloalkyl, optionally substituted C6-C10 aryl, and optionally substituted C7-C12 arylalkyl, preferably t-butyldimethylsilyl (TBDMS), triethylsilyl (TES), t-butyldiphenylsilyl (TBDPS), triisopropylsilyl (TIPS), and trimethylsilyl (TMS); or tetrahydropyranyl (THP), methoxymethyl (MOM), benzyloxymethyl;
ethoxyethyl, benzyl or tert-butyl; phenoxyacetate (pac), tert-butylphenoxyacetate (tac), benzoate or substituted benzoate groups; carbonates, preferably benzyloxycarbonate (CBZ), tert-butyloxycarbonate (BOC), fluorenylmethoxycarbonate (FMOC); and acetals, preferably methoxymethyl (MOM), 2-methoxyethoxymethyl (MEM), benzyloxymethyl, or tetrahydropyranyl (THP); and
wherein R³ and R⁴ may be the same or different and are selected from the group consisting of hydrogen, methyl, ethyl, n-propyl, isopropyl, trimethylsilyl, or R3 and R⁴ taken together with the intervening nitrogen atom form an optionally substituted heterocyclyl such as pyrrolidine, piperidine, morpholine, 2,2,6,6-tetramethylpiperidine, imidazole, 4,5-dichloroimidazole, triazole, and tetrazole.

61. The process according to claim 60, wherein in compound A and in compound B R³ are R⁴ are isopropyl groups.

62. The process according to any one of claims 60-61, wherein R¹ and R² are TBDMS.

63. The process according to any one of claims 60-62, wherein steps A and/or B are carried out in one or more polar aprotic solvents, preferably the solvent system comprises DMF, toluene or a mixture thereof.

64. The process according to any one of claims 60-63, wherein steps A and/or B are carried out in the presence of an activator.

65. The process according to claim 64, wherein the activator is a Lewis acid, acidic heterocycle, a salt of an organic base, an organic acid or an inorganic acid.

66. The process of claim 65, wherein the activator is a salt of a nitrogen containing heterocycle, preferably pyridinium trifluoroacetate or N-methylimidazolium trifluoroacetate.

67. The process according to any one of claims 60-66, wherein compound B is prepared by a process comprising reacting compound 7 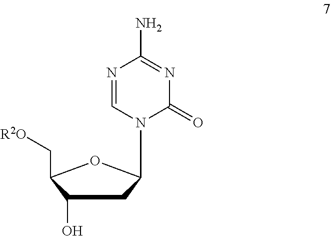
with compound C: 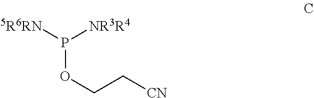
wherein R¹ and R² are as defined above, R³, R⁴, R⁵ and R⁶ may be the same or different and are selected from the group consisting of hydrogen, methyl, ethyl, n-propyl, isopropyl, trimethylsilyl, or R³ and R⁴ or R⁵ and R⁶ taken together with the intervening nitrogen atom form an optionally substituted heterocyclyl, preferably R³, R⁴, R⁵ and R⁶ are isopropyl.

68. The process according to any one of claims 60-67, wherein compound A is prepared by a process comprising reacting compound 4 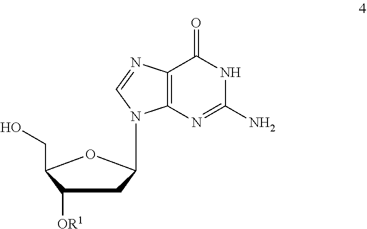
with compound C: 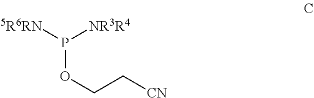
wherein R¹ and R² are as defined above, R³, R⁴, R⁵ and R⁶ may be the same or different and are selected from the group consisting of hydrogen, methyl, ethyl, n-propyl, isopropyl, trimethylsilyl, or R³ and R⁴ or R⁵ and R⁶ taken together with the intervening nitrogen atom form an optionally substituted heterocyclyl, preferably R³, R⁴, R⁵ and R⁶ are isopropyl.

69. The process according to any one of claims 60-68 wherein compound C is produced in situ in situ from compound D: 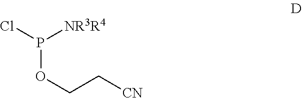
wherein R³ and R⁴ are the same as for compound C above.

70. The process according to any one of claims 60-69, wherein R² is TBDMS.

71. The process according to any one of claims 60-70, wherein the reaction is carried out in the presence of a solvent, preferably DMF and an activator, preferably N-methylimidazolium trifluoroacetate.

72. The process according to any one of claims 60-71, wherein the process further comprises converting compound 10, preferably wherein R¹ and R² are the same and are TBDMS, to Guadecitabine.

73. The processes according to any one of claims 45-59, wherein compound 10 is prepared according to any one of claims 60-72.

74. A compound selected from the group consisting of compounds 5, 7, 9, 10 and 11, 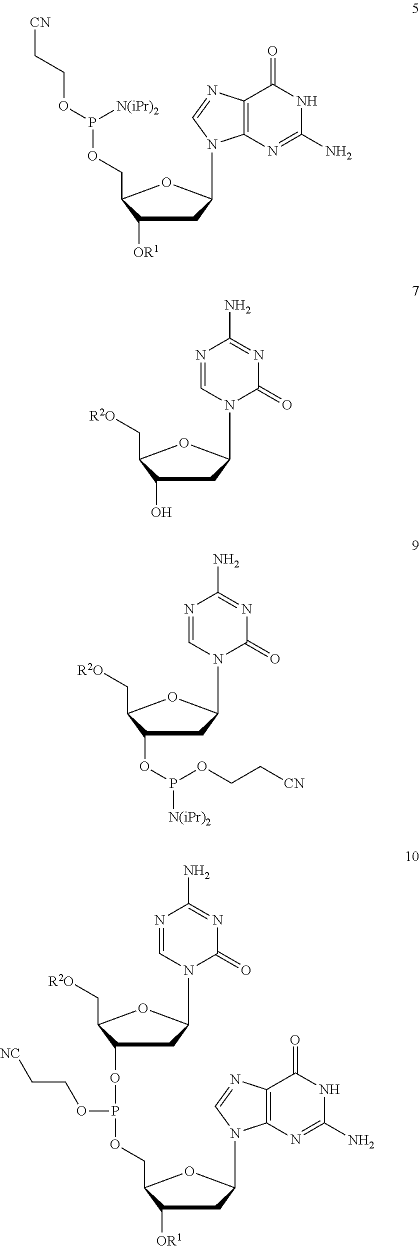 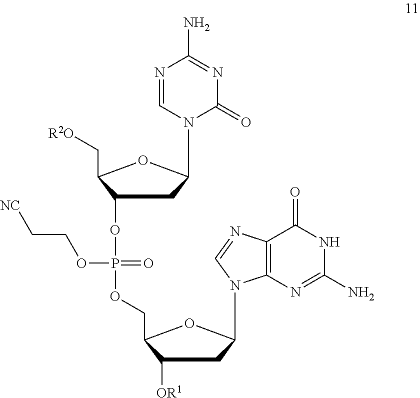
wherein R¹ and R² may be the same or different suitable hydroxy protecting group, preferably selected from —SiR⁷R⁸R⁹, wherein R⁷, R⁸ and R⁹ are independently selected from the group consisting of: C1-C15 straight or branched alkyl, C1-C10 cycloalkyl, optionally substituted C6-C10 aryl, and optionally substituted C7-C12 arylalkyl, preferably t-butyldimethylsilyl (TBDMS), triethylsilyl (TES), t-butyldiphenylsilyl (TBDPS), triisopropylsilyl (TIPS), and trimethylsilyl (TMS); or tetrahydropyranyl (THP), methoxymethyl (MOM), benzyloxymethyl; ethoxyethyl, benzyl or tert-butyl; phenoxyacetate (pac), tert-butylphenoxyacetate (tac), benzoate or substituted benzoate groups; carbonates, preferably benzyloxycarbonate (CBZ), tert-butyloxycarbonate (BOC), fluorenylmethoxycarbonate (FMOC); and acetals, preferably methoxymethyl (MOM), 2-methoxyethoxymethyl (MEM), benzyloxymethyl, or tetrahydropyranyl (THP).

75. The compound of claim 74, wherein R¹ and R² are the same and are TBDMS.

76. Use of the compound according to any one of claims 74-75 in the preparation of Guadecitabine or a salt thereof.

77. The compound according to any one of claims 74-75 for use in the preparation of guadecitabine or salt thereof.

78. A process for preparation of Guadecitabine or salts thereof, preferably guadecitabine sodium, comprising:
a) reacting compound 4, preferably wherein R¹ is TBDMS (compound 4a) 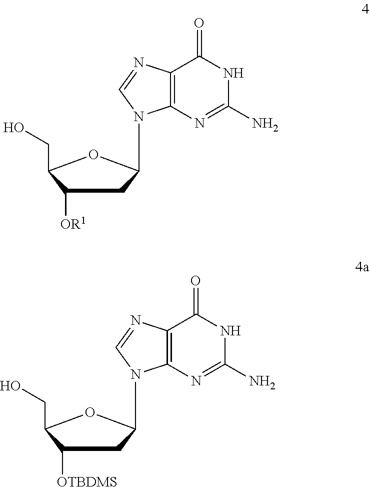
with compound B, preferably wherein R³ and R⁴ are isopropyl (compound 9), more preferably wherein R³ and R⁴ are isopropyl and R² is TBDMS (compound 9a) 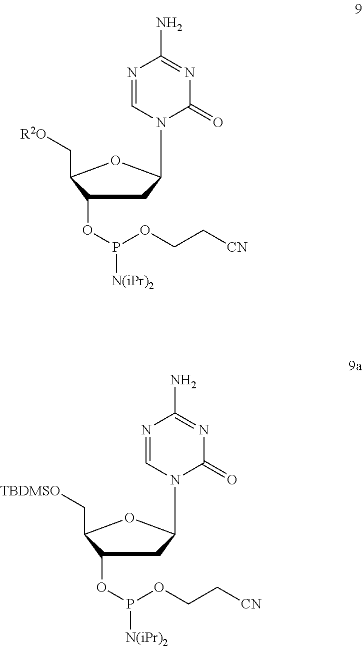
to provide compound 10, preferably wherein R¹ and R² are TBDMS (compound 10a); 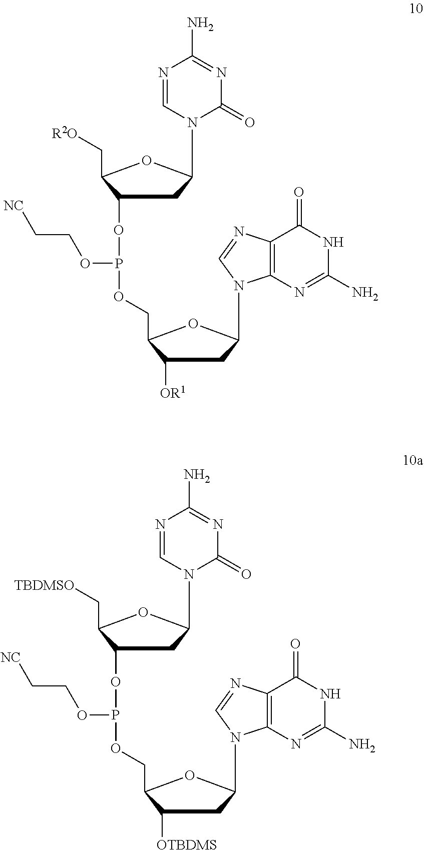
b) oxidizing compound 10, preferably wherein R¹ and R² are TBDMS (compound 10a); to afford compound 11, preferably wherein R¹ and R² are TBDMS (compound 11a) 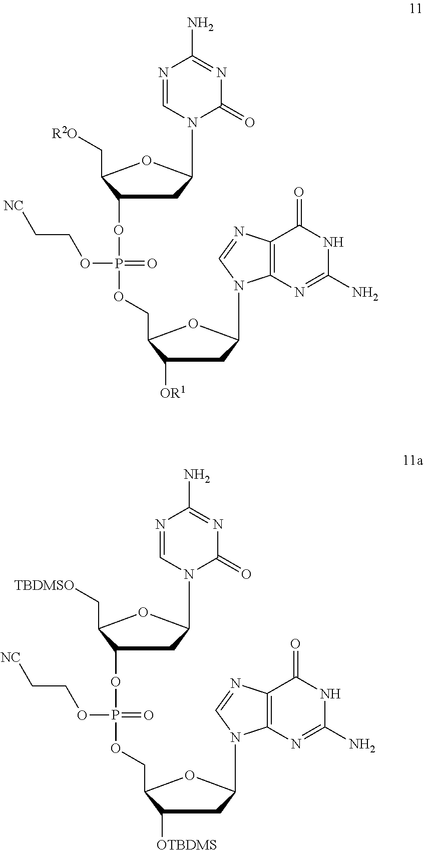
c) converting the compound produced in step b) to Guadecitabine or salt thereof, preferably Guadecitabine sodium.

79. The process according to claim 78, wherein step c) comprises:
i) deprotecting compound 11 or preferably 11a in the presence of a suitable base, a fluoride reagent and a solvent wherein the base is triethylamine and wherein the solvent comprises DMA and DMSO to afford a triethylammonium salt of Guadecitabine;
ii) providing the triethylammonium salt of Guadecitabine in a mixture of an alcohol and water, preferably methanol and water, more preferably wherein the wherein the amount of water is NMT about 50% v/v, NMT about 20% v/v, NMT about 10% v/v, or NMT about 5% v/v, preferably wherein the amount of water is NMT about 40% v/v, more preferably NMT about 30% v/v, and most preferably from about 10% v/v to about 30% v/v;
iii) adding a guanidine salt, preferably guanidine carbonate, more preferably a solution of guanidine carbonate in water and optionally adding an antisolvent to afford Guadecitabine guanidinium, preferably in crystalline form;
iv) providing the Guadecitabine guanidinium in a mixture of an alcohol and water, preferably methanol and water, more preferably wherein the amount of water is NMT about 50% v/v, NMT about 20% v/v, NMT about 10% v/v, or NMT about 5% v/v, preferably wherein the amount of water in the solvent system is NMT about 30% v/v, more preferably NMT about 20% v/v, even more preferably NMT about 10% v/v, and most preferably from about 5% v/v to about 10% v/v; and
v) adding a sodium salt, preferably sodium acetate, more preferably a solution of guanidine carbonate in methanol and optionally adding an antisolvent to afford Guadecitabine sodium, preferably in crystalline form.

80. Guadecitabine or salt thereof, preferably Guadecitabine sodium, prepared according to any one of claims 21-59 or 78-79.

81. A pharmaceutical composition comprising Guadecitabine sodium according to any one of claims 1-7 or 80.

82. Guadecitabine or salt thereof, preferably Guadecitabine sodium according to any one of claims 1-7 or 80 or the pharmaceutical composition according to claim 81 for use in therapy.

83. Guadecitabine or salt thereof, preferably Guadecitabine sodium according to any one of claims 1-7 or 80 or the pharmaceutical composition according to claim 81 for use in the treatment of cancer.

84. Use of Guadecitabine or salt thereof, preferably guadecitabine sodium according to any one of claims 1-7 or 80 in the preparation of a medicament for the treatment of cancer.

85. A method of treating cancer comprising administering a therapeutically effective amount of pharmaceutical composition according to claim 81.

86. A process for preparation of a pharmaceutical composition according to claim 81 comprising combining the solid forms of Guadecitabine sodium according to any one of claims 1-7 or 80 with at least one pharmaceutically acceptable excipient.

87. The crystalline form of Guadecitabine sodium according to any one of claims 1-3 or 7, or the pharmaceutical composition according to claim 81 for use in therapy.

88. The crystalline form Guadecitabine sodium according to any one of claims 1-3 or 7, or the pharmaceutical composition according to claim 81 for use in the treatment of cancer.

89. A method of treating cancer comprising administering a therapeutically effective amount of pharmaceutical composition according to claim 81.
